(19) United States
(12) Patent Application Publication
(10) Pub. No.: US 2011/0033430 A1
(43) Pub. Date: Feb. 10, 2011

(54) METHODS FOR THE INDUCTION OF A CELL TO ENTER THE ISLET 1+ LINEAGE AND A METHOD FOR THE EXPANSION THEREOF

(75) Inventors: Kenneth R Chien, Cambridge, MA (US); Yibing Qyang, San Diego, CA (US); Silvia Martin-Puig, Cambridge, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/526,447

(22) PCT Filed: Feb. 8, 2008
(86) PCT No.: PCT/US08/53449

§ 371 Date: Sep. 14, 2009

Related U.S. Application Data
(60) Provisional application No. 60/900,496, filed on Feb. 9, 2007.

(51) Int. Cl. A61K 35/12; C12N 5/071; C12N 5/10; A61P 9/00

(52) U.S. Cl. 424/93.21; 435/377; 435/325; 424/93.7

(57) ABSTRACT

The present invention relates to methods for the induction and a cell to enter the Islet 1⁺ (Isl1⁺) lineage and methods for expansion of cells of islet 1⁺ lineage. One aspect of the present invention relates to methods to induce a cell to enter the islet 1⁺ lineage, and more particularly to a method to induce a cell to enter a the Isl1⁺ lineage to become an Isl1⁺ progenitor that is capable of differentiating along multiple different lineages such as a endothelial lineage, a smooth muscle lineage or a cardiac lineage. In particular, one embodiment present invention relates to methods to induce a cell to enter the Isl1⁺ lineage by inhibiting a wnt signalling pathway in the cell. Another aspect of the present invention relates to methods to expand a cell of the Isl1⁺ lineage, such as a Isl1⁺ progenitor by activating a wnt signalling pathway in the Isl1⁺ progenitor. Another aspect of the present invention relates to use of cells of the isl1⁺ lineage in subjects for therapeutic and preventative treatment of cardiovascular diseases.

### CROSS REFERENCED APPLICATION

This application claims benefit under 35 U.S.C 119(e) of U.S. Provisional Application Ser. No. 60/900,496 filed on Feb. 9, 2007 the contents of which are incorporated herein in their entirety by reference.

### BACKGROUND OF THE INVENTION

Cardiogenesis requires the formation of a diverse spectrum of muscle and non-muscle cell lineages in specific tissue compartments in the heart. Understanding how embryonic precursor cells generate and control the formation of distinct endothelial, pacemaker, atrial, ventricular, and vascular smooth muscle lineages, as well as how these cells become positioned to form the specific chambers, aorta, coronary arteries, and conduction system in the heart, is of fundamental importance in unravelling the developmental logic and molecular cues that underlie both cardiovascular development and disease.

Recent studies employing a combination of in vivo lineage tracing and clonal analyses have resulted in the discovery of a subset of multipotent islet cardiovascular progenitors that can ultimately give rise to all three of these major cell types in the heart. The progeny of these cells eventually contribute to most of the major components of the heart, such as the atrium right ventricle and septum, aorta, pulmonary artery, coronary artery, and the sinus node and conduction system.

However, one of the major limitations in using these progenitors for cardiovascular regenerative medicine relates to the difficulty of markedly expanding well-defined clonal cardiovascular progenitor cell populations, from either intact human tissue, or ES cell based systems. In particular, the feasibility of utilizing human ES cells as a source for differentiated cardiac myocytes has related largely to the inability to markedly enhance the process of in vitro cardiogenesis, as less than 1% of the differentiated progeny enter cardiac lineages.

Therefore there is great need in the art for methods that efficiently enable the formation of cardiovascular progenitors and maintaining and expanding these cardiovascular progenitors in a multipotent state to enable the generation of a diverse set of heart lineages. A desired method would enable the production of, and subsequent unlimited expansion of progenitors capable of entering different cardiac lineages. Such a method is highly desirable as it will circumvent many of the issues relating to tissue rejection commonly associated with cell-based transplantation therapies, as cells from a subject obtained, induced to become a desired cell lineage, expanded and subsequently reintroduced into the subject to treat a variety of disorders, for example cardiac and cardiovascular disorders, without the effectiveness of the therapy being compromised by tissue rejection.

### SUMMARY OF THE INVENTION

The present invention relates to methods for the production and expansion of isl1⁺ progenitors, for example cardiovascular progenitors, while maintaining their multipotency and capacity for multi-lineage differentiation. The present invention is based, in part, on the discovery that wnt signals directly affect the occurrence of islet 1⁺ progenitors and their renewal.

The inventors have discovered that a cardiac mesenchymal cell (CMC) feeder layer utilizes a paracrine wnt/β-catenin signaling pathway to titrate the number of isl1⁺ progenitors. The inventors have discovered that in one instance the wnt/β-catenin signaling pathway negatively regulates the formation of isl1⁺ progenitors, and in another instance wnt/β-catenin signaling pathway triggers the renewal of isl1⁺ progenitors.

The inventors have discovered that by suppressing wnt signaling, they are able to trigger the induction of cells to become isl1⁺ progenitors. Furthermore, the inventors have also discovered that by activating wnt signaling pathway once cells have entered the isl1⁺lineage, they are able trigger renewal of isl1⁺ progenitors. Accordingly, the present invention provides methods for (i) inducing a cells to enter the islet 1⁺ lineage and production of isl1⁺ progenitors, and (ii) renewal of isl1⁺ progenitors enabling the large scale expansion and production of isl1⁺ progenitors, for example large scale production of isl1⁺ cardiovascular progenitors. The inventors have discovered, by using the methods described herein, that they are able to generate and expand isl1⁺ progenitors, for example isl1⁺ cardiovascular progenitors, while maintaining their multi-lineage differentiation capacity. These progenitors can be further directed to differentiate along specific lineages. For example, the inventors demonstrate methods to induce a human ES cell to become an isl1⁺ progenitor, in particular an isl1⁺ cardiovascular progenitor, which can then be subsequently renewed using the methods provided herein, while maintaining its multi-lineage differentiation potential. In some embodiments, such isl1⁺ progenitors, in particular isl1⁺ cardiovascular progenitors derived by the methods as disclosed herein can be subsequently differentiated, for example isl1⁺ progenitors can be differentiated along cardiac lineages and towards specific heart tissue components that have immediate clinical therapeutic value.

In one aspect, the present invention provides to a method to induce a cell to enter the islet 1⁺ lineage. In particular, the present invention is based on the discovery of suppression or inhibition of wnt signaling triggers the entry of cells into islet 1+ lineage and can be used to pre-specify cells to become isl1⁺ progenitors. Accordingly, the present invention provides methods to induce cells, for example uncommitted progenitors, for example but not limited to, mesodermal progenitors into isl1⁺ progenitors, for example isl1⁺ cardiovascular progenitors.

In a second aspect, the present invention provides a method to expand isl1⁺ progenitors by triggering their renewal. The method provided herein enable renewal of isl1⁺ progenitors in absence of cell feeder layer. In particular, the present invention is based on the discovery that activation of the wnt pathway can trigger the expansion and renewal of the isl1⁺ progenitors. Accordingly, the present invention also provides methods to expand isl1⁺ cells in a feeder-free system, for example in a cardiac mesenchymal cell (CMC) free system.

In one embodiment, the present invention provides methods for inducing a cell to enter the islet 1+ lineage by inhibiting or suppressing the wnt/β-catenin pathway. In some embodiments, the cell is a progenitor, in some instance the progenitor is an uncommitted progenitor, for example, a mesoderm progenitor. In alternative embodiments, the cell is a stem cell, including, but not limited to, an embryonic stem cell, embryoid body (EBs), adult stem cell and a fetal or postnatal stem cell. In some embodiments, the cell is obtained from tissue. In some embodiments, the tissue is, for example embryonic, fetal, postnatal or adult tissue. In some embodiments the tissue is cardiac tissue. In some embodiments, the tissue includes, but is not limited to, fibroblasts, cardiac fibroblasts, circulating endothelial progenitors, pancreas, liver, adipose tissue, bone marrow, kidney, bladder, palate, umbilical cord, amniotic fluid, dermal tissue, skin, muscle, spleen, placenta, bone, neural tissue or epithelial tissue. In some embodiments, the cell is from a subject with a disease or disorder, for example from a subject with an acquired or congenital cardiac or cardiovascular disorder, disease or dysfunction, for example a cardiac or heart defect.

In some embodiments, the cell is mammalian and in some embodiments, the cell is human. In some embodiments, the cell is a rodent cell. In some embodiments the cell is a genetically modified cell.

In some embodiments of the present invention, inhibition and/or suppression of the wnt/β-catenin pathway is by wnt inhibitory agents. In some embodiments, wnt inhibitory agents are directly applied to the cell, for example wnt inhibitory agents are applied to culture media in which the cell is maintained, and in some embodiments the cell is cultured in the presence of a cell feeder layer, for example a cardiac mesenchymal cell (CMC) feeder layer. In alternative embodiments, nucleic acids encoding wnt inhibitory agents are expressed by the cell and/or a cell feeder layer, for example a cardiac mesenchymal cell (CMC) feeder layer.

In some embodiments, a wnt inhibitory agent inhibits the expression and/or activity of the gene or gene product encoding Wnt or Wnt3, or homologues thereof. In some embodiments, a wnt inhibitory agent inhibits the expression and/or activity of Wls/Evi or homologues thereof. In alternative embodiments, a wnt inhibitory agent is any component of the wnt/β-catenin pathway, for example but not limited to Dickkopf-1 (DKK1), WIF-1, cerberus, secreted frizzled-related proteins (sFRP), sFRP-1, sFRP-2, collagen 18 (collagen XVIII), endostatin, carboxypeptidase Z, receptor tyrosine kinase, corin, and Dgl or homologues, genetically modified versions and fragments of these. In some embodiments, a wnt inhibitory agent is a peptide agonist of DKK1. In some embodiments, a wnt inhibitory agent increases the expression and/or activates GSK, for example GSK-3β. In some embodiments, more than one wnt inhibitory agent is used, and any combination of wnt inhibitory agents can be used; In some embodiments, the administration of one or more wnt inhibitory agents is by different means, for example, one wnt inhibitory agent is administered to the culture medium, and another wnt inhibitory agent is encoded by a nucleic acid in the cell and/or cell feeder layer.

In another aspect of the present invention, methods to expand isl1⁺ progenitors (i.e., progenitors that are already isl1⁺) by activating or enhancing the wnt/β-catenin pathway are provided. In particular, the present invention is based on the discovery that increasing or enhancing wnt signaling triggers renewal of isl1⁺ progenitors and can be used to expand isl1⁺ progenitors while maintaining their capacity for multi-lineage differentiation. Accordingly, provided herein are methods to expand isl1⁺ progenitors, for example isl1⁺ cardiovascular progenitors. In some embodiments, the methods of the present invention enable expansion of isl1⁺ progenitors, for example isl1⁺ cardiovascular progenitors in a feeder-free system, and in alternative embodiments, the methods of the present invention provide for the enhanced expansion of isl1⁺ progenitors in the presence of a cell feeder layer.

In some embodiments, the isl1⁺ progenitor is obtained from a cell by the methods described herein. In alternative embodiments, the isl1⁺ progenitor is obtained by any means commonly known by persons of ordinary skill in the art. In some embodiments, the isl1⁺ progenitor is of mammalian origin, and in some embodiments, the isl1⁺ progenitor is human. In some embodiments, the isl1⁺ progenitor is a genetically modified isl1⁺ progenitor. In some embodiments, the isl1⁺ progenitor is a isl1⁺ cardiovascular progenitor, and in some embodiments the isl1⁺ progenitor also expresses Nkx2.5 and flk1.

In some embodiments of the present invention, activation of or enhancing the wnt/β-catenin pathway is affected by wnt activating agents. In this context, wnt activating agents are any agents which activate the wnt/β-catenin pathway. Preferably, such agent(s) activate the wnt/β-catenin pathway in a selective manner. In some embodiments, the wnt activating agents are directly applied to the isl1⁺ progenitor, for example, wnt activating agents are applied to the progenitor's culture media. In alternative embodiments, nucleic acids encoding wnt activating agents are expressed by the isl1⁺ progenitor and/or a cell feeder layer, for example a cardiac mesenchymal cell (CMC) feeder layer.

In some embodiments, a wnt activating agent is, and/or activates the expression and/or activity of, the gene or gene product encoding Wnt or Wnt3, or homologues or genetically modified versions thereof that are active in wnt pathway signaling. In other embodiments, a wnt activating agent is, and/or activates the expression and/or activity of the gene or gene product encoding β-catenin, or homologues or genetically modified versions thereof that activate or participate in the wnt signaling pathway.

In alternative embodiments, wnt activating agents suppress or inhibit the activity or expression of inhibitors or suppressors of the wnt/β-catenin pathway. For example, a wnt activating agent is an inhibitor of GSK, for example an inhibitor of GSK-3 and/or inhibitor of GSK-3β. GSK proteins are inhibitors of the wnt pathway. Thus, their inhibition can activate wnt signaling. In some embodiments, an inhibitor of GSK3 is for example, but is not limited to, 6-bromoindirubin-3′-oxime (BIO) or analogues and mimetics thereof. In some embodiments, analogues of BIO are for example, acetoxime analogue of BIO or Azakenpaulline or analogues thereof. In some embodiments, an inhibitor of GSK3 is a peptide inhibitor for GSK3, for example, a peptide inhibitor of GSK3 with SEQ ID NO: 5.

In some embodiments, an isl1⁺ progenitor generated by the methods of the present invention, and/or isl1⁺ progenitors expanded by the methods of the present invention are cryopreserved. In some embodiments, the cell is cryopreserved before it is subjected to wnt inhibitory agents, and in some embodiments, the cell is cryopreserved after it has entered the islet 1⁺ lineage. In some embodiments, the isl1⁺ progenitors are cryopreserved before they are subjected to wnt activating agents, and in some embodiments they are cryopreserved after they are subjected to wnt activating agents.

In some embodiments, the wnt inhibitory agents and/or wnt activating agents include nucleic acid, protein, small molecule, antibody and aptamer agents or wnt-activating analogues or fragments thereof. In some embodiments the wnt inhibitory agents and/or wnt activating agents are nucleic acids encoding a protein or fragment thereof, small inhibitory nucleic acid molecules, siRNA, shRNA, miRNA, antisense oligonucleic acids (ODNs), PNA, DNA or nucleic acid analogues. In some embodiments, nucleic acids are nucleic acid analogues, for example but not limited to peptide nucleic acid (PNA), pseudo-complementary PNA (pcPNA), and locked nucleic acid (LNA) and analogues thereof.

In another aspect, methods are provided that use the isl1⁺ progenitors generated and expanded by the methods described herein. In one embodiment, the isl1⁺ progenitors generated and expanded by the methods described herein are used for the production of a composition, for example a composition for regenerative medicine. In some embodiments, the composition is for use in transplantation into subjects in need of cardiac transplantation, for example but not limited to subjects with congenital and/or acquired heart disease and/or subjects with vascular diseases and/or cardiovascular diseases. In some embodiments, the isl1⁺ progenitors generated and expanded by the methods of the present invention may be genetically modified. In another aspect, the subject may have or be at risk of heart disease and/or vascular disease and/or cardiovascular disease. In some embodiments, the isl1⁺ progenitors generated and expanded by the methods of the present invention may be autologous and/or allogenic. In some embodiments, the subject is a mammal, and in other embodiments the mammal is a human.

In another aspect of the invention, the isl1⁺ progenitors generated and expanded by the methods of the present invention are used in assays. In some embodiments, the assays are used for screening agents, for example agents for the development of therapeutic interventions of diseases, including, but not limited to, therapeutics for congenital and adult heart failure. In alternative embodiments the isl1⁺ progenitors generated and expanded by the methods of the present invention are used in assays to for screening agents that are toxic to the cell, for example the isl1⁺ progenitors generated and expanded by the methods herein can be used in cardiotoxicity assays.

### BRIEF DESCRIPTION OF FIGURES

The patent or application file contains at least one drawing executed in color. Copies of this patent or patent application publication with color drawings(s) will be provided by the Office upon request and payment of the necessary fee.

FIGS. 1A-1C shows the in vivo localization of murine and human neonatal isl1⁺ cardiovascular progenitors. FIG. 1A shows a schematic diagram of genetic marking of isl1⁺ cardiovascular progenitors by genetic crossing experiments. FIGS. 1B and 1C show frozen sections of hearts which were obtained from neonatal mice isl1-mER-Cre-mER/R26R. Cre-mediated recombination and results in a selective lacZ expression and genetic marking of isl1 expressing cells at the time of tamoxifen injection. Shown in FIG. 1B is a section of the outflow tract (OFT) area, and show in FIG. 1C is the region adjacent to right atrium (RA) in the dorsal-anterior direction after X-gal staining (black staining) and immuno-histochemical analysis for cardiac troponin T (gray staining). Circles indicate isl1⁺ clusters in a non-cardiomyocyte compartment. Arrows designate β-gal⁺ cells and insets represent a magnification of the areas of interest. Scale bar, 200 μm.

FIGS. 2A-2P show the identification and characterization of a chemical probe (BIO) that augment the expansion of postnatal isl1⁺ cardiovascular progenitors from a high-throughput chemical screening. FIG. 2A shows a schematic diagram of the high-throughput chemical screening assay. Tamoxifen injection in isl1-mER-Cre-mER/R26R double heterozygous mice followed by administration of 4-OH-TM in culture leads to the expression of lacZ in isl1⁺ progenitors. These were then treated with compounds from a chemical library and lysed to determine β-gal activities, quantified by luminescent signal. FIG. 2B shows β-gal activity correlates with the number of isl1⁺ progenitors in postnatal cardiac mesenchymal cells (1K=1,000 cell). FIG. 2C shows BIO (6-bromoindirubin-3′-oxime) and two unknown compounds (Comp A and Comp B) which were identified from a chemical library to significantly increase luciferase activity. Mean values±SEM, n=3 to 8, *p<0.05, **p<0.01, ***p<0.001. FIGS. 2D-2P show the effect of BIO on the expansion of postnatal isl1⁺ cardiovascular progenitors. Isl1+ expression is show in FIGS. 2D and 2E without BIO (the control) and FIGS. 2F and 2G show Isl1+ expression in the BIO-treated sample. Insets in 2F and 2G show a magnification of isl1⁺ cells. Nuclei were detected with Hoechst dye (2E and 2G). Scale bar, 50 mm. FIG. 2H shows the quantification of the effect of BIO, FIG. 2I shows the effect of 1-Azakenpaullone, and FIG. 2J shows the effect of an acetoxime analog of BIO, and FIG. 2K shows the effect of a GSK-3 peptide inhibitor treatment at different doses on the expansion of postnatal isl1⁺ cardiovascular progenitors. Mean±SEM, n 3. Note quantification of each treatment represents the total number of isl1⁺ cells per culture. FIGS. 2L-2P shows Human neonatal cardiac tissue-derived cells were cultured in the presence of either DMSO (control) as shown in FIGS. 2L and 2M, or BIO as shown in FIGS. 2N and 2O and stained for isl1. FIG. 2P shows quantification of the effect of BIO on the expansion of postnatal human isl1⁺ cardiovascular progenitors, mean±SEM, n=6. Scale bar, 25 mm. *p<0.05, **p<0.01, ***p<0.001.

FIGS. 3A-3O shows the Wnt/β-catenin pathway plays a pivotal role in the control of the expansion of postnatal isl1⁺ cardiovascular progenitors. FIGS. 3 A and 3B show the effect of Wnt3a on the expansion of postnatal isl1⁺ cardiovascular progenitors, as shown by quantification for the number of isl1⁺ cells as shown in FIG. 3A and isl1⁺ clusters, as shown in FIG. 3B, as detected by Isl1 immunostaining. (Mean values±SEM, n=3, ***p<0.001). FIGS. 3C and 3D shows the effect of Dkk1 on the expansion of postnatal isl1⁺ cardiovascular progenitors, as shown by quantification for the number of isl1⁺ cells (as shown in FIG. 3C) and isl1⁺ clusters, as shown in FIG. 3D which is detected by Isl1 immunostaining. (Mean values±SEM, n=3, **p<0.01, ***p<0.001). FIG. 3E shows a schematic diagram of the Wnt signalling indicator mouse strain, TOPGAL, in which the production of β-galactosidase represents an active wnt/β-catenin signaling. FIG. 3F-3O show immunofluorescent analysis for Isl1 and cytoplasmic β-galactosidase from the sections of an ED 10.5 TOPGAL heart. FIG. 3F shows a low magnification, and FIGS. 3G-3M show a high magnification of the section from the outflow tract (OFT) area, whereas FIGS. 3J-3L show the left atrial (LA) region (N-P) after Isl1 and β-gal staining. White arrows** mark double positive cells. Scale bars, 200 inn (FIG. 3J) and 100 inn (FIGS. 3G-3L). FIGS. 3M-30 show immunostaining for Isl1 and β-catenin in postnatal cardiac mesenchymal cells. **White arrows mark isl1⁺ cells costaining for nuclear β-catenin. **Yellow arrows indicate isl1⁺ cells with an accumulation of cytoplasmic β-catenin. Scale bar, 100 μm.

FIGS. 4A-4D show the CMC feeder layer and the wnt/β-catenin ligand expand the multipotent isl1⁺ anterior heart field cells. FIG. 4A show a schematic diagram of the purification, expansion and differentiation of the anterior heart field isl1⁺ cells. FIG. 4B shows RT-PCR showing that isl1 expression segregates to the GFP positive population in CMC expanded anterior heart field Isl1⁺ cells. FIG. 4C shows FACS analysis showing that GFP⁺/Isl1⁺ cells expanded on CMC treated with BIO have greater expansion of GFP⁺/Isl1⁺ cells than DMSO control, and GFP⁺/Isl1⁺ cells exposed to Wnt3a conditioned media have significantly greater expansion compared to control media. FIG. 4D shows expanded isl1⁺ progenitors maintain their ability to differentiate into smooth muscle cells and cardiomyocytes. GFP⁺/Isl1⁺ anterior heart field cells were expanded on CMC layers treated with the conditions shown, and put into differentiation conditions for smooth muscle or cardiomyocytes. The number of cells positive for smooth muscle myosin heavy chain (SM-MHC) or cardiac Troponin-T were then scored and compared to controls.

FIGS. 5A-5T show wnt/β-catenin Pathway Plays a Pivotal Role in the Control of the Expansion of Isl1⁺ Cardiovascular Progenitors. FIGS. 5A and 5B shows Isl1+ immunofluorescence on a control, and FIGS. 5C and 5D show Isl1+ immunofluorescence on a Wnt3a-producing feeder layer. Arrows point to isl1⁺ cells. Asterisks indicate feeder layer cells. Scale bar, 25 mm. FIG. 5E shows quantification of the number of isl1⁺ cells detected by immunostaining on a Wnt3a feeder layer compared with control. Mean values±SEM, n=6, ***p<0.001. FIGS. 5F and 5G show Isl1 immunofluorescence analysis of embryonic E8.5 isl1⁺ progenitors on a control feeder layer, whereas FIGS. 5H and 5I show isl1+ immunostaining on a Wnt3a-producing feeder layer. Scale bar, 50 mm. FIGS. 5J and 5K show flow cytometry profile of E8.5 cells from AHF enriched tissue of double transgenic isl1-IRES-Cre; Z/RED embryos after expansion on a control or Wnt3a feeder layer for 7 days. FIG. 5L shows quantification of the number of dsRed⁺ progenitors on Wnt3a versus control feeder. Mean values±SEM, n=3, ***p<0.001. FIGS. 5M to 5O show dsRed signal (FIG. 5N) correlates highly with isl1 expression (FIG. 5M) in cells from double transgenic embryos expanded on Wnt3a feeder layer. Scale bar, 25 mm. FIG. 5P shows the spontaneous differentiation of dsRed⁺ progenitors into smooth muscle cells, revealed by expression of SMA and FIG. 5Q shows spontaneous differentiation of dsRed⁺ progenitors into smooth muscle cells as detected by the expression of SM-MHC. FIG. 5R shows differentiation of ds-Red⁺ progenitors can be driven by coculture with neonatal murine cardiomyocytes. Scale bar: 25, 50, and 25 mm, respectively. FIG. 5S shows a schematic representation of the experimental procedure for isolation, expansion and purification of DsRed-tagged embryonic isl1⁺ cardiovascular progenitors FIG. 5T shows the frequency of spontaneous differentiation of DsRed⁺ progenitors into SMC-MHC after 7 days in culture. Mean values±SEM, n=5.

FIGS. 6A-6F show the expansion of Isl1⁺ AHF Cells by a Wnt3a Feeder Layer. FIG. 6A shows a schematic diagram of the AHF construct, AHF-GFP transgenic mouse, and embryonic stem cell derivation strategy. FIG. 6B shows FACS profile of EB day 6 differentiated AHF-GFP ES cells. FIGS. 6C and 6D show cTnT and SM-MHC staining of sorted EB day 6 GFP⁺ cells. Scale bar, 25 mm. FIG. 6E shows quantitative PCR analysis showing the isl1 and mef2c expression levels normalized by GAPDH in freshly sorted GFP⁺ and GFP_cells from EB day 6 differentiated AHF-GFP ES cells. Mean±SD, n=3.

FIGS. 7A-7P show the pre-specification, Expansion and Differentiation of Isl1+ Cardiovascular progenitors by the wnt/β-catenin Pathway. FIG. 7A shows a schematic representation of the experimental strategy. FIGS. 7B-7D show Wnt3a treatment markedly inhibits the formation of MICPs. FIG. 7B shows the control, and FIG. 7C shows Wnt3a-conditioned medium which was added to the coculture for 24 hr, and single β-gal⁺ cells were scored after X-gal staining. FIG. 7D shows a bar graph which shows the mean values±SEM, n=5, ***p<0.001. FIGS. 7E-7G show Dkk1 treatment significantly promotes the formation of MICPs. FIG. 7E shows the control, and FIG. 7F shows Dkk1-conditioned medium which was added to the coculture for 24 hr, and single b-gal⁺ cells were scored after X-gal staining. FIG. 7G shows a bar graph which shows the mean values±SEM, n=3, *p<0.05. Scale bar, 50 mm. FIG. 7H-7M shows the effects of the wnt/β-catenin pathway on the expansion of prespecified MICPs. Single EB-derived precursors were plated on CMC and allowed to grow for 3 days. FIG. 7I shows Wnt3a and FIG. 7L shows Dkk1 conditional media or their respective controls (shown in FIGS. 7H and 7K) which were then added to the coculture for an additional 3 days prior to the assessment of β-Gal⁺ colonies. FIGS. 7J and 7M show bar graphs showing the mean values±SEM, n=3. *p<0.05, ***p<0.001. Scale bars, 100 mm. FIGS. 7N-7R show Wnt3a inhibits cardiomyocyte differentiation of isl1⁺AHF cells. AHF-GFP⁺ cells sorted on EB day 6 were plated on fibronectin-coated slides in the presence of Wnt3a, as shown in FIG. 7O, or control (See FIG. 7N)—conditioned media. FIG. 7P shows that, following fixation and cTnT immunostaining, the total number of cTnT⁺ cells per well was scored. FIG. 7Q shows AHF-GFP ES cells which were sorted on EB day 6, and GFP cells which were plated on control feeder layers or cells stably transfected with Wnt3a (as shown in FIG. 7R) followed by immunostaining.

FIGS. 8A-8I show abnormal OFT Morphology, Disrupted OFT Myocardial Differentiation, and Marked Expansion of Isl1⁺ Pharyngeal Mesodermal Progenitors in Murine Embryos that Harbor a Constitutive Activation of β-catenin within AHF Lineages. FIGS. 8AA-C′ show the anatomical morphology of the heart in a control (FIGS. 8A-8C) and a mutant (β-cat[ex3]_{AHF} [A′-C′]) E9.5 embryo. The head and pharyngeal arches 1 to 2 were removed to allow an optimal view of the heart components. LV, left ventricle; RA, right side of the primary atrium; LA, left side of the primary atrium. Scale bars, 500 mm. FIGS. 8D-8F′ show the coronal sections through the OFT of a control (8D-8F) and a mutant (8D′-8F′) E9.5 embryo immunostained for isl1 and SMA. Boxed areas are magnified on the right of the row. The yellow arrows indicate isl1⁺; sma⁺ cells, and the white arrows indicate isl1⁺; sma_cells. The cutting plane at the medial part of the OFT is indicated on the schematic heart image. Scale bars: 25 mm in (FIGS. 8D and 8D′), 50 mm in (FIG. 8E-8F′). FIGS. 8G-8H′ show sagittal sections of a control (8G and 8H) and a mutant (b-cat[ex3]_{AHF})(8G′ and 8H′) E9.5 embryo immunostained for isl1 and pi-H3. FIGS. 8G and 8G′ show the cutting planes at the medial, and FIGS. 8H and 8Hi show lateral part of the embryos are indicated on the schematic heart image. The isl1⁺ cardiac progenitor population between the cardiac OFT and IFT is outlined with orange dashed lines. The boxed area in each panel is magnified on the top-left corner. I, II, III: first, second, and third pharyngeal arches; A, medial part of the primary atrium. Scale bars, 100 mm. FIGS. 8I and 8I′ show 3D reconstruction of isl1⁺ pharyngeal mesoderm between the cardiac OFT and IFT from serial sections (represented by the areas outlined by orange dashed lines in FIG. 8G to FIG. 8H′). The control is represented in green and the mutant in red. Shown are ventral (1 and 10), dorsal (2 and 20), and left (3 and 30) views of the reconstructed structures.

FIG. 9 shows decreased Proliferation of the OFT Myocardial Cells in Murine Embryos with a Temporally Controlled Loss of Function of β-catenin. FIG. 9 shows quantification of proliferating myocardial cells in the OFT of control and mutant embryos. Immunostaining of pi-H3, was performed on transverse sections of a control (Isl1-MCM^{/+}; β-cat^{+/f}) and a mutant (Isl1-MCM^{/+}; β-cat_{—}^{/f}) embryo (E11.5). Tamoxifen was injected to pregnant females at E9.5, and embryos were harvested at E11.5. Nuclei were identified by DAPI staining (data not shown) and the inventors identified and quantified the proliferating myocardial cells in OFT and proliferating endocardial cells in OFT. Mean±SEM, n=3, ***p<0.001.

FIGS. 10A-10B shows two models of the Effects of wnt/β-catenin Signaling on the Renewal and Differentiation of Isl1⁺ Cardiac Progenitor Cells and Their Progenies. FIG. 10A shows an in vivo model. In the AHF of wild-type embryos, wnt/β-catenin signaling promotes the proliferation of isl1⁺ cardiac progenitors, which are negative for sma. The progenitors migrate to the OFT myocardium and undergo stepwise differentiation. While the cells in the distal part of the OFT start to express sma, they remain positive for isl1. In contrast, in the proximal part of the OFT isl1 expression is lost in a considerable portion of the cells. In the b-cat(ex3)AHF mutant, with augmented wnt/β-catenin signaling in the AHF and its derivatives, there is an increased proliferation of the isl1⁺ cardiac progenitors in the AHF but inhibited differentiation of the progenitors and their progenies after they migrate to the myocardial layer of the OFT. FIG. 10B shows a model of the roles of wnt/β-catenin signals from CMC feeder on the pre-specification, renewal, and differentiation of a hierarchy of isl1⁺ cardiovascular progenitors, demonstrating that the cardiac mesenchymal niche differentially controls the pre-specification and renewal of isl1+ cardiovascular progenitors via a paracrine wnt/β-catenin pathway.

FIGS. 11A-11C show the induction of human ES (hES) cells to become islet 1+ progenitors and their subsequent expansion using human ISL1-βgeo BAC Transgenic ES cell lines. FIG. 11A shows the cassette construct of βgeo gene under the control of ISL1 locus. The βgeo reporter gene was introduced into Isl1 locus in human BAC clone CTD-2314G24, which contains all exons of human Isl1 gene and extends from 100.7 kb upstream to 26.1 kb downstream of the translational start site. βgeo=β-galactosidase and neomycin-resistance fusion protein; BAC=human Bacteria Artificial Chromosome CTD-2314G24. FIGS. 11B and 11C show human ES cells comprising ISL1-βgeo BAC that express Isl1 can be identified by β-galactosidase staining. Human ES cells shown are at Embryonic Body E6 (EB6) differentiation Stage.

FIGS. 12A-12F show enrichment of human Isl1+ progenitor cells. FIG. 12A show a schematic of enrichment and isolation of stem cells using cardiac mesenchymal cell (CMC) feeder layer. ISL1-βgeo BAC transgenic hEBs are in suspension culture for 5 days, then dissociated and plated on mouse cardiac mesenchymal fibroblast cells for additional 2 days. FIG. 12B shows β-Gal expression in Islet-1⁺ progenitors from hES cells. X-gal staining (BF) which identifies Lac-Z expressing cells is detected in the cytoplasm, and FIG. 12C shows Islet-1 (ISL1) immunostaining is detected in the nucleus. FIG. 12D-12F shows immunostaining of human stem cells derived from single cell of hEBs cultured on tissue-specific mesenchymal feeder layer. FIG. 12D shows anti-ISL1 immunostaining which is detected in the nucleus and FIG. 12E shows anti-LacZ (β-geo) immunostaining which is detected in the cytoplasm (shown by the FIG. 12F which shows the merged image).

FIGS. 13A-13J show the detection of Islet-1 positive stem cells from hEB cultured on mesenchymal feeder layer. FIGS. 13A and 13D show X-gal staining (BF) which identifies Lac-Z expressing cells is detected in the cytoplasm, and FIG. 13B show Islet-1 (ISL1) immunostaining is detected in the nucleus, as identified by co-staining with DAPI (data not shown), with the merged images shown in FIGS. 13C and 13F respectively. FIG. 13G-13J show individual colonies of dissociated and plated islet-1+ hES cells cultured on CMC for additional 5-7 days, showing renewal of human isl 1⁺ progenitors.

FIG. 14A-14C shows isl1+ progenitors on inhibition of the wnt/β-catenin pathway. FIG. 14A a schematic of the methods of siRNA transfection of wnt3a secreting CMC. FIG. 14B shows siRNAs (siWLS-A (SEQ ID NO:1) and siWLS-B (SEQ ID NO:2)) against murine Wls/Evi significantly decreased its activity toward secreting Wnt3a in an co-culture system (**p<0.01, ***p<0.001). Wnt-reporter cells were co-cultured with Wnt3a-secreting cells, transfected with siRNAs. Luciferase activities were then measured to assess the efficacy of siRNAs from two experiments. FIG. 14C shows siWLS-A (SEQ ID NO:1) treatment significantly increased the formation of MICPs, which was quantified as shown in FIG. 14D. Single EB-derived precursors were plated on siRNA or control-treated CMC feeder layers for 24 hrs and single β-gal⁺ cells were then scored. Mean values±SEM, n=3, ***p<0.001. Scale bar, 100 μm.

### DETAILED DESCRIPTION

### I. Overview

The present invention relates to the discovery that wnt/β-catenin signaling is both a negative and positive regulator of Islet 1⁺ (isl1⁺) progenitors. Without wishing to be bound by theory, the inventors have discovered that by specific manipulation of the wnt/β-catenin signaling pathway it is possible to recreate the appropriate microenvironment niche for cells to form islet 1 positive (isl1⁺) progenitors, for example isl1⁺ cardiovascular progenitors from uncommitted progenitors, and that different manipulation of the wnt/β-catenin signaling pathway enables the subsequent renewal and expansion of isl1+ progenitors.

By using high-throughput screening of progenitors, the inventors have discovered a method to reconstitute the cardiac mesenchymal niche, which is normally provided by a cardiac mesenchymal cell (CMC) feeder layer. This cardiac mesenchymal niche is required for renewal of isl1⁺ progenitors, for example isl1⁺ cardiovascular progenitors. Accordingly, the inventors have discovered a method for the renewal of isl1⁺ progenitor cells, for example isl1⁺ cardiovascular progenitor in a feeder-free system.

Using loss of function and gain of function studies, the inventors have also discovered that a cardiac mesenchymal cell layer exerts inhibitory signals preventing cells from entering the isl1+ lineage pathway and forming isl1⁺ progenitors, for example isl1⁺ cardiovascular progenitors from uncommitted progenitors. The inventors discovered that these inhibitory signals that prevent cells from entering the isl1⁺ lineage are part of the wnt/β-catenin signaling pathway. Without wishing to be bound by theory, the inventors have discovered that canonical wnt ligands suppress (i.e. negatively regulate) positive signals from the cardiac mesenchymal cell feeder layer that triggers cells to enter the isl1⁺ progenitor lineage. Accordingly, the inventors have discovered that by inhibiting wnt signaling they can induce cells, for example uncommitted progenitor cells, to enter the isl1⁺ lineage pathway and become isl1⁺ progenitors. Accordingly, the inventors have discovered a method to induce uncommitted progenitor cells to enter the Islet 1 lineage pathway and become isl1⁺ progenitors, for example isl1⁺ cardiovascular progenitors by inhibiting wnt signaling.

Stated another way, the inventors have importantly discovered that the cardiac mesenchymal cell (CMC) feeder layer utilizes a paracrine wnt/β-catenin signaling pathway to carefully titrate the number of isl1⁺ progenitors. In one instance, the CMC-derived wnt/β-catenin signaling pathway negatively regulates the formation of isl1⁺ progenitors by inhibiting the positive signals from the CMC feeder layer that cue cells to enter the isl1⁺ lineage pathways. Effectively, the inventors have discovered that wnt/β-catenin signaling dictates if a cell, for example an uncommitted progenitor, will enter the islet 1⁺ lineage pathway. Furthermore, the inventors have also discovered that by inhibiting the wnt/β-catenin pathway, they can trigger a cell, for example an uncommitted progenitor, to enter the islet 1⁺ lineage pathway.

In another instance, the inventors have discovered that once a cell is an isl1⁺ progenitor, for example, an isl1⁺ cardiovascular progenitor, the CMC-derived wnt/β-catenin signaling pathway triggers their renewal while maintaining their capacity for multi-lineage differentiation, for example differentiation towards more committed lineages downstream of the isl1⁺ cardiovascular progenitor hierarchy, such as for example, cardiovascular vascular progenitors and cardiovascular muscle progenitors and subsequently cardiac, smooth muscle and endothelial cell lineages.

Accordingly, the present invention provides methods for; (i) triggering cells, for example uncommitted progenitors to enter the islet 1⁺ lineage pathway by inhibiting the wnt/β-catenin pathway, and (ii) expanding isl1⁺ progenitors, for example any isl1⁺ progenitor of the isl1⁺ cardiovascular progenitor hierarchy, by activating the wnt/β-catenin pathway. In some embodiments of the invention, the methods to induce cells to enter the islet 1⁺ lineage pathway by inhibiting the wnt/β-catenin pathway occurs in the presence of CMC, and in alternative embodiments it occurs in the absence of CMC. In other embodiments of the invention, the methods for renewal of isl1⁺ progenitors by activating the wnt/β-catenin pathway occurs in the absence of CMC, therefore the present invention provides methods for renewal and expansion of isl1⁺ progenitors in a feeder-free system. In other embodiments, renewal of isl1⁺ progenitors by activating the wnt/β-catenin pathway occurs in the presence of CMC.

One aspect of the invention relates to the inhibition and/or suppression of the wnt signaling pathway to induce the differentiation of cells into isl1⁺ progenitors. Another aspect of the invention relates to the activation or enhancement of the wnt signaling pathway to induce the renewal or proliferation of isl1⁺ progenitors, for example isl1⁺ cardiovascular progenitors. The invention provides methods for the formation of isl1⁺ progenitors, for example isl1⁺ cardiovascular progenitors in a feeder-free system. The invention also provides methods for the renewal and proliferation of isl1⁺ progenitors, for example isl1⁺ cardiovascular progenitors in the absence of a feeder layer.

Another embodiment of the invention provides methods for the induction of progenitors to enter the islet 1 lineage pathway and the formation of isl1⁺ progenitors by inhibiting the wnt/β-catenin pathway. In some embodiments, one or more agent is used to inhibit or suppress component of the wnt/β-catenin pathway, herein termed “wnt inhibitory agents” or “inhibitory agents”. In some embodiments wnt inhibitory agents inhibit wnt or homologues thereof, for example but not limited to wnt and wnt3a inhibitory nucleic acids, for example but not limited to wnt and/or wnt3a RNAi and WLS/Evi RNAi. In some embodiments, wnt inhibitory agents are endogenous inhibitors and/or activate expression or activity of endogenous inhibitors of wnt/β-cateinin signaling, for example but not limited to, DKK1, Dapper, WIF-1, secreted frizzled-related proteins (sFRP), sFRP-1, sFRP-2, sRFP-1, sRFP-2, cerbertus, collagen 18, endostatin, carboxypeptidase Z, receptor tyrosine kinase, corin, dgl, pertussis toxin, disabled-2 (dab-2), Fezb, FrzA, sizzled etc and homologues and variants thereof. In another embodiment, wnt inhibitory agents can be any agent that inhibits and/or suppresses the wnt/β-catenin pathway, for example Axin and LRP lacking the intracellular domain.

In alternative embodiments, the wnt inhibitory agents inhibit β-catenin, for example β-catenin nucleic acid inhibitory molecules, such as β-catenin RNAi etc. In other embodiments, wnt inhibitory agents can be agents inhibiting β-catenin, for example, but not limited to protein phosphatase 2 (PP2A), chibby, ponrin 52, Nemo/LNK kinases, and HMG box factors such as XSox17 and HBP1 and homologues thereof.

In further embodiments, wnt inhibitory agents can activate endogenous inhibitors of wnt/β-catenin signaling. As a non-limiting example, wnt inhibitory agents can activate GSK, for example, GSK-3β. Examples of GSK-3β activators are any agent, for example a peptide and/or gene of GSK-3β, or agents that activate the PKB-pathway, including, but not limited, to wortamanin. GSK-3β activators are known by the skilled artisan and are disclosed in U.S. Patent US2003/0114382, which is incorporated herein in its entirety by reference.

In alternative embodiments, the invention provides methods for the expansion and renewal of isl1⁺ progenitors by activating the wnt/β-catenin pathway. In some embodiments, one or more agent is used to activate or enhance the wnt pathway, herein termed “wnt activating agents” or “activating agents”. In some embodiments wnt activating agents directly activate wnt, for example, direct activation of wnt, wnt3a or homologues thereof, for example agents that increase the expression and/or activity of wnt3a and homologues thereof, for example peptides of Wnt3a or fragments or variants thereof. In some embodiments, the agents activate wnt-related peptides, for Wnt1, Wnt2, Wnt3B/Wnt13, Wnt3, Wnt3A, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16 or a bioactive fragment thereof or a wnt polypeptide that promotes wnt signaling via the canonical wnt or wnt/β-catenin, which are disclosed in U.S. Pat. Nos. 5,851,984 and 6,159,462, which are incorporated herein in their entirety by reference. In additional embodiments, wnt activators can include agents such as, for example but not limited to WLS peptide, PAR1 kinase, disheleved (Dsh), Dally (division abnormally delayed) and dally-like and LRP, for example LRP-5 and LRP-6.

In other embodiments, wnt activating agents can be any agent that activates the wnt/β-catenin pathway through activation of β-catenin, for example but not limited to Frodo, TCF, Pitx2, Pertin 52, lef-1, legless (lgs), pygopus (pygo), hyrax/parafibromin and homologues thereof.

In other embodiments, wnt activating agents can be any agent that inhibits the activity of components which suppress the wnt/β-canetin-GSK3 pathway, for example, wnt activating agents can inhibit GSK, for example GSK3β. In some embodiments, wnt activating agents which inhibit GSK-3β include, but are not limited to, 6-bromoindirubin-3′-oxime (BIO), BIO analogues, for example acetoxime analogue of BIO or 1-Azakenpaulline or analogues or mimetics thereof that inhibit GSK. GSK-3β inhibitors are commonly known by the person of ordinary skill in the art, and include, for example lithium and LiCl, retinoic acid and estradiol, and are disclosed in International Patent WO97/41854, which is incorporated herein in its entirety by reference.

### II. Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “progenitor cells” is used herein to refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. It is possible that a cell can begin as progenitor cell and can proceed toward a differentiated phenotype, but then “reverse” and re-express the progenitor cell phenotype, thus a progenitor cell can be derived from a non-stem cell.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art.

The terms “cardiovascular progenitor” or “cardiovascular stem cell” and “cardiac stem cell” are used interchangeably herein, and refer to a stem cell which is capable of proliferation and giving rise to more progenitor cells having the ability to generate a large number of mother cells that can in turn give rise to differentiated, or differentiable daughter cells which can eventually terminally differentiate into cardiac cells, cardiovascular cells and other cells of the cardiovascular system.

The term “multipotent isl1⁺ cardiovascular progenitors” and “Isl1⁺ cardiovascular progenitor” and “MICP” are used interchangeably herein, refer to the population of cardiovascular progenitors with the transcriptional signature profile of isl1/nkx2.5/flk-1 which are capable of differentiating into and generating the three major cell types in the heart; cardiac, smooth muscle and endothelial cells. MICP cells have been cloned from both mouse embryonic stem cells and mouse embryos, and human ES cells and can make this decision at a single cell level, and represent a cell lineage at the highest level of a hierarchical lineage pathway similar to a hematopoietic paradigm. Multipotent isle cardiovascular progenitors are disclosed in U.S. Provisional Patent application 60/856,490 and International Patent Application No: PCT/US07/23155 and Moretti et al., 2006, which are incorporated herein in their entirety by reference.

The term “differentiation” in the present context means the formation of cells expressing markers known to be associated with cells that are more specialized and closer to becoming terminally differentiated cells incapable of further division or differentiation. The pathway along which cells progress from a less committed cell, to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that in the context of this application, the term “differentiation” or “differentiated” refers to cells that are more specialized in their fate or function than at a previous point in their development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as progressive differentiation or progressive commitment.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The terms “mesenchymal cell” or “mesenchyme” are used interchangeably herein and refers to in some instances the fusiform or stellate cell's found between the ectoderm and endoderm of young embryos; most mesenchymal cell's are derived from established mesodermal layers, but in the cephalic region they also develop from neural crest or neural tube ectoderm. Mesenchymal cells have a pluripotential capacity, particularly embryonic mesenchymal cells in the embryonic body, developing at different locations into any of the types of connective or supporting tissues, to smooth muscle, to vascular endothelium, and to blood cells. A mesenchymal stem cell refers to a cell from the immature embryonic connective tissue.

The terms “mesenchymal progenitor” or “mesodermal progenitors”, also known as “MSCs”, are used interchangeably herein, refer to progenitor cells of mesodermal origin. The mesoderm is the middle embryonic germ layer, lying between the ectoderm and the endoderm, from which connective tissue, muscle, bone, and the urogenital and circulatory systems develop.

The term “lineages” as used herein describes a cell with a common ancestry or cells with a common developmental fate. In the context of a cell that has entered an “islet 1+ lineage” this means the cell is an Islet 1+ progenitor and expresses Islet 1+, and can differentiate along the Isl1+ progenitor lineage restricted pathways, such as one or more developmental lineage pathways such an endothelial lineage, a cardiac lineage or a smooth muscle lineage as these terms are defined herein. For example, a cell that has entered the Isl1+ lineage is a cell which is capable of differentiating into three major cell types in the heart; cardiac, smooth muscle and endothelial cells. For reference, methods to identify a cell which is of islet1+ lineage is disclosed in U.S. Provisional Patent application 60/856,490 and International Patent Application No: PCT/US07/23155 which are incorporated herein in their entirety by reference.

As used herein, the term “clonal cell line” refers to a cell lineage derived from a single cell that can be maintained in culture and has the potential to propagate to produce daughter cells. A clonal cell line can be a stem cell line or be derived from a stem cell, and where the clonal cell line is used in the context of clonal cell line comprising stem cells, the term refers to stem cells which have been cultured under in vitro conditions that allow proliferation without differentiation for months to years. Such clonal stem cell lines can have the potential to differentiate along several lineages of the cells from the original stem cell.

A “marker” as used herein is used to describe the characteristics and/or phenotype of a cell.

Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

The term “phenotype” refers to one or a number of total biological characteristics that define the cell or organism under a particular set of environmental conditions and factors, regardless of the actual genotype.

The term “tissue” refers to a group or layer of specialized cells which together perform certain special functions. The term “tissue-specific” refers to a source of cells from a specific tissue.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are not isl1+ progenitor or their progeny as defined by the terms herein. In some embodiments, the present invention provides methods to expand a population of isl1⁺ progenitors, wherein the expanded population of isl1⁺ progenitors is a substantially pure isl1⁺ progenitor population.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

As used herein, the term “cardiovascular disease, condition or disorder” is defined as a medical condition related to the cardiovascular (heart) or circulatory system (blood vessels). By way of background, a response to myocardial injury follows a well-defined path in which some cells die while others enter a state of hibernation where they are not yet dead but are dysfunctional. This is followed by infiltration of inflammatory cells and deposition of collagen as part of scarring, all of which happen in parallel with in-growth of new blood vessels and a degree of continued cell death. The term cardiovascular diseases is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging, includes, but is not limited to, diseases and/or disorders of the pericardium, heart valves (i.e., incompetent valves, stenosed valves, rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Yet further, one skilled in the art recognizes that cardiovascular diseases can result from congenital defects, genetic defects, environmental influences (i.e., dietary influences, lifestyle, injury, stress, etc.), and other defects or influences, and combinations thereof.

The term “disease” or “disorder” is used interchangeably herein, refers to any alteration in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, in disposition or affliction.

The term “pathology” as used herein, refers to symptoms, for example, structural and functional changes in a cell, tissue, or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology-causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, associated with other factors, for example ischemia and the like.

As used herein, the term “cardiovascular tissue” is defined as heart tissue and/or blood vessel tissue.

As used herein, the term “coronary artery disease” (CAD) refers to a type of cardiovascular disease. CAD is caused by gradual blockage of the coronary arteries. One of skill in the art realizes that in coronary artery disease, atherosclerosis (commonly referred to as “hardening of the arteries”) causes thick patches of fatty tissue to form on the inside of the walls of the coronary arteries. These patches are called plaques. As a plaque thickens, the artery narrows and blood flow decreases, which results in a decrease in oxygen to the myocardium. This decrease in blood flow precipitates a series of consequences for the myocardium. For example, interruption in blood flow to the myocardium results in an “infarct” (myocardial infarction), which is commonly known as a heart attack.

As used herein, the term “damaged myocardium” refers to myocardial cells that have been exposed to ischemic conditions. These ischemic conditions may be caused by a myocardial infarction, or other cardiovascular disease or related complaint. The lack of oxygen causes the death of the cells in the surrounding area, leaving an infarct, which eventually scars.

As used herein, the term “infarct” or “myocardial infarction (MI)” refers to an interruption in blood flow to the myocardium. Thus, one of skill in the art refers to MI as death of cardiac muscle cells resulting from inadequate blood supply.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue

As used herein, the term “myocardium” refers to the muscle of the heart.

The term “regeneration” means regrowth of a cell population, organ or tissue after disease or trauma.

The term “agent” refers to any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “wnt activating agent” refers any agent that activates the wnt/β-catenin pathway, or inhibits or suppresses the activity of inhibitors of wnt/β-catenin pathway, for example inhibitors of GSK-3β activity. The activation is preferably selective activation, which means that the wnt3 pathway is activated to the substantial exclusion of the effects (i.e. activation of inhibition) on non-wnt3 pathways. By way of a non-limiting example, BIO is shown to selectively activate the wnt3 pathway which is demonstrated by the dose-dependent expansion of Isl1+ progenitors as shown in Example 2. A wnt activating agent as used herein can activate the wnt/β-catenin pathway at any point along the pathway, for example, but not limited to increasing the expression and/or activity of wnt, wnt dependent genes and/or β-catenin, and decreasing the expression and/or activity of endogenous inhibitors of wnt and/or β-catenin or inhibitors of components of the wnt/β-catenin pathway. For example a non-limiting example, prohibiting phosphorylation of β-catenin leads to an accumulation of β-catenin and an association of β-catenin with TCF/LEF and/or an increase in the expression and/or activity of Wnt dependent genes.

As used herein, the term “wnt inhibiting agent” refers to any agent that inhibits the wnt/(3-catenin pathway, or enhances the activity and/or expression of inhibitors of wnt/β-catenin signaling, for example activators or enhancers of GSK-3β activity. A wnt inhibitory agent as used herein can suppress the Wnt/β-catenin pathway at any point along the pathway, for example, but not limited to decreasing the expression and/or activity of wnt, or β-catenin or wnt dependent genes and/or proteins, and increasing the expression and/or activity of endogenous inhibitors of wnt and/or β-catenin or increasing the expression and/or activity of endogenous inhibitors of components of the wnt/β-catenin pathway, for example increasing the expression of GSK-3β.

As used herein, the term “GSK-3” means the enzyme glycogen synthase kinase 3 and homologs or functional derivatives thereof. As discussed herein, GSK-3 is conserved among organisms across the phylogenetic spectrum, although the homologs present in various organisms differ in ways that are not significant for the purposes of the present invention. One of skill in the art will appreciate that the present invention may be practiced using any of the eukaryotic homologs of GSK-3. Furthermore, vertebrate GSK-3 exists in two isoforms, denoted GSK-3α and GSK-3β. GSK-3α and GSK-3β differ from one another only in ways that are not significant for the purposes of the present invention. Therefore, the terms “GSK-3”, “GSK-3α”, and “GSK-3β” are used interchangeably herein. Although the preferred embodiment of the present invention and the examples presented herein exemplify the study and use of GSK-3β, the invention should not be considered to be limited to this particular isoform of GSK-3.

Thus, nucleic acid compositions encoding wnt, β-catenin, or GSK-3β amino acid sequences are herein provided and are also available to a skilled artisan at accessible databases, including the National Center for Biotechnology Information's GenBank database and/or commercially available databases, such as from Celera Genomics, Inc. (Rockville, Md.). Also included are splice variants that encode different forms of the protein, if applicable. The nucleic acid sequences may be naturally occurring or synthetic.

As used herein, the terms “wnt, β-catenin, and/or GSK-3β nucleic acid sequence,” “wnt, β-catenin, and/or GSK-3β polynucleotide,” and “wnt, β-catenin, and/or GSK-3β gene” refer to nucleic acids described herein, homologs thereof, and sequences having substantial similarity and similar function, respectively. A skilled artisan recognizes that the sequences are within the scope of the present invention if they encode a product which regulates at least one of the following functions, activation of the wnt/-catenin signaling pathway, activation of Wnt dependent genes, accumulation of β-catenin, inhibition of phosphorylation of β-catenin, increased expression of cardiac specific transcription factors or genes, and furthermore knows how to obtain such sequences, as is standard in the art.

Thus, one of skill in the art recognizes that the agents of the present invention modulate Wnt signal transduction at any point along the known wnt/β-catenin pathway, or yet undiscovered pathway, including but not limiting to induction of cells along isl1⁺ progenitor differentiation pathway and proliferation and renewal of isl1⁺ progenitors, association of proteins with transcription factors and/or cardiac specific genes, increasing or decreasing expression and/or activity of enzymes, increasing or decreasing expression and/or activity of Wnt dependent genes or proteins, increasing or decreasing expression and/or activity of known activators or inhibitors or yet undiscovered activators or inhibitors of wnt and/or for β-catenin and/or wnt dependent genes, increasing or decreasing the expression and/or activity of known activators or yet undiscovered activators of wnt and/or, β-catenin and/or wnt dependent genes, etc., or increasing or decreasing the expression and/or activity of known inhibitors or yet undiscovered inhibitors of wnt and/or, β-catenin and/or wnt dependent genes, etc

As used herein, the term “therapeutically effective amount” refers to an amount that results in an improvement or remediation of the disease, disorder, or symptoms of the disease or condition.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total). Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis.

As used herein, the terms “treat” or “treatment” or “treating” as used herein in the context of treatment of cardiac disorder or enhancing cardiac function, refers to both therapeutic treatment and prophylactic or preventative measures, wherein the object is to prevent or slow the development of the disease, such as slow down the development of a cardiac disorder, or reducing at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

As used herein, the terms “administering,” “introducing” and “transplanting” are used interchangeably in the context of the placement of isl1⁺ progenitors, for example isl1⁺ cardiovascular stem cells of the invention into a subject, by a method or route which results in at least partial localization of the cardiovascular stem cells at a desired site. The cardiovascular stem cells can be administered by any appropriate route which results in delivery to a desired location in the subject where at least a portion of the cells or components of the cells remain viable. The period of viability of the cells after administration to a subject can be as short as a few hours, e.g. twenty-four hours, to a few days, to as long as several years.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the central nervous system, such that it enters the animal's system and, thus, is subject to metabolism and other like processes, for example, subcutaneous administration.

As used herein, the term “DNA” is defined as deoxyribonucleic acid.

A “reporter gene” as used herein encompasses any gene that is genetically introduced into a cell that adds to the phenotype of the stem cell. Reporter genes as disclosed in this invention are intended to encompass fluorescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

The term “recombinant” as used herein with reference to material (e.g., a cell, a nucleic acid, a protein, or a vector) indicates that such material has been modified by the introduction of a modified or heterologous genetic material. Thus, for example, recombinant microorganisms or cells express one or more genes that are not found within the native (non-recombinant) form of the microorganism or cell or express native genes that are otherwise abnormally expressed, under expressed or not expressed at all. For example, a recombinant antibody is an antibody which is not normally found in native (non-recombinant) antibody forms, expressed from a manipulated coding sequence.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

As used herein, the term “tissue-specific promoter” means a nucleic acid sequence that serves as a promoter, i.e., regulates expression of a selected nucleic acid sequence operably linked to the promoter, and which selectively affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause lesser expression in other tissues as well.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in suspending, maintaining the activity of or carrying or transporting the subject agents from one organ, or portion of the body, to another organ, or portion of the body. Each carrier must be “acceptable” in the sense of being compatible with the other ingredients of the formulation. Pharmaceutically acceptable carriers include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions. A pharmaceutically acceptable carrier will not promote an immune response to an agent which it carries.

The term “amino acid” within the scope of the present invention is used in its broadest sense and is meant to include naturally occurring L α-amino acids or residues, but is not necessarily restricted to the naturally occurring amino acids.

The terms “gene(s)” refers to a nucleic acid sequence that encodes through its template or messenger RNA a sequence of amino acids characteristic of a specific peptide. The term “gene” can include intervening, non-coding regions, as well as regulatory regions, and can include 5′ and 3′ ends.

The term “gene product(s)” as used herein refers to RNA transcribed from a gene, or a polypeptide encoded by a gene or translated from RNA.

The term “homologue” or “homologous” as used herein refers to homology with respect to structure and/or function. With respect to sequence homology, sequences are homologs if they are at least 50%, preferably at least 60%, more preferably at least 70%, more preferably at least 80%, more preferably at least 90%, more preferably at least 95% identical, more preferably at least 97% identical, or more preferably at least 99% identical. The term “substantially homologous” refers to sequences that are at least 90%, more preferably at least 95% identical, more preferably at least 97% identical, or more preferably at least 99% identical. Homologous sequences can be the same functional gene in different species.

The term “analog” as used herein refers to an agent that retains the same biological function (i.e., binding to a receptor) and/or structure as the polypeptide or nucleic acid it is an analogue of. Examples of analogs include peptidomimetics (a peptide analog), peptide nucleic acids (a nucleic acid analog), small and large organic or inorganic compounds, as well as derivatives and variants of a polypeptide or nucleic acid herein.

The term “derivative” or “variant” as used herein refers to a peptide, chemical or nucleic acid that differs from the naturally occurring polypeptide or nucleic acid by one or more amino acid or nucleic acid deletions, additions, substitutions or side-chain modifications, yet retains one or more specific functions or activities of the naturally occurring molecule. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative.

The term “substantially similar”, when used to define either a wnt, β-catenin, and/or GSK-3β amino acid sequence or wnt, β-catenin, and/or GSK-3β nucleic acid sequence, means that a particular subject sequence, for example, a mutant sequence, varies from the sequence of the natural (or wild-type) wnt, β-catenin, and/or GSK-3β, respectively, by one or more substitutions, deletions, or additions, the net effect of which is to retain at least some of the biological activity found in the native natural wnt, β-catenin, and/or GSK-3β protein, respectively. As such, nucleic acid and amino acid sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence. A nucleotide sequence is “substantially similar” to a specific nucleic acid sequence as disclosed herein if: (a) the nucleotide sequence is hybridizes to the coding regions of the natural wnt, β-catenin, and/or GSK-3β gene, respectively; or (b) the nucleotide sequence is capable of hybridization to nucleotide sequence of wnt, β-catenin, and/or GSK-3β under moderately stringent conditions and wnt, β-cateninin, and/or GSK-3β, respectively having biological activity similar to the native proteins; or (c) the nucleotide sequences which are degenerative as a result of the genetic code to the nucleotide sequences defined in (a) or (b). Substantially similar proteins will typically be greater than about 80% similar to the corresponding sequence of the native protein.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” refers to hybridization under stringent conditions, high stringent conditions, low stringent conditions or moderately stringent conditions as those conditions are commonly known by persons of ordinary skill in the art.

The terms “reduced”, “reduction” or “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “xenogeneic” refers to cells that are derived from different species.

The articles “a” and “an” are used herein to refer to one or to more than one (i.e., to at least one) of the grammatical object of the article. By way of example, “an element” means one element or more than one element.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%. The present invention is further explained in detail by the following examples, but the scope of the invention should not limit thereto.

### III. The Wnt/β-Catenin Signaling Pathway

Without wishing to be bound by theory, Wnt proteins and their cognate receptors signal through at least two distinct intracellular pathways. The “canonical” Wnt signaling pathway, (referred to herein as the wnt/β-catenin pathway) involves wnt signaling via β-catenin to activate transcription through TCF-related proteins (van de Wetering et al. (2002) Cell 109 Suppl: S13-9; Moon et al. (2002) Science 296(5573): 1644-6). A non-canonical alternative pathway exists, in which wnt activates protein kinase C(PKC), calcium/calmodulin-dependent kinase II (CaMKII), JNK and Rho-GTPases (Veeman et al. (2003) Dev Cell 5(3): 367-77), and is often involved in the control of cell polarity.

In brief and without wishing to be bound by theory, the wnt initiates wnt/β-catenin signaling by binding with Frizzled receptors of cell surfaces. When the wnt proteins bind with the Frizzled receptors, GSK-3 (glycogen synthase kinase-3) is inactivated (phosphorylated), preventing GSK-3 from decomposing β-catenin, and thus, β-catenin accumulates in the cytoplasm. The accumulated beta-catenin is translocated into cell nuclei and induces transcription of various genes together with a Lef/TCF transcription factor and stimulates the expression of genes including c-myc, c-jun, fra-1, and cyclin D1. This pathway is called “wnt/β-catenin signaling”.

Wnt/β-catenin signaling can also be induced by drugs that directly inactivate the GSK-3 enzymes, such as lithium, retinoic acid, or BIO, in addition to the wnt proteins.

Wnt signaling via Frizzled receptors is mediated by co-receptor low-density lipoprotein receptor related proteins (LRP5, LRP6), (called arrow in Drosophila) (Wehrli et al. (2000) Nature 407(6803): 527-30; Tamai et al. (2000) Nature 407(6803): 530-5; Pinson et al. (2000) Nature 407(6803): 535-8) to mediate signaling via dishevelled (dsh).

Wnt/β-catenin signaling also requires the DIX domain of ash; deletion of this domain strongly inhibits Wnt signals via β-catenin (Tada and Smith. (2000) Development 127(10): 2227 38). The kinase PAR1 interacts with dishevelled and is a positive regulator of wnt-β-catenin signaling (Sun et al. (2001) Nat Cell Biol 3(7): 628-36). The dishevelled binding protein Frodo is also an essential positive regulator of Wnt/β-catenin signals (Gloy et al. (2002) Nat Cell Biol 4(5): 351-7). Dsh is negatively regulated by naked cuticle (naked) (Zen et al. (2000) Nature 403(6771): 789-95; Rousset et al. (2001) Genes Dev 15(6): 658-71) and Dapper (Cheyette et al. (2002) Dev Cell 2(4): 449-61). Disabled-2 (dab-2) interacts with both Dvl and Axin, and functions as a negative regulator of wnt/β-catenin signaling (Hocevar et al. (2003) EMBO J 22(12): 3084-94). LKB1/XEEK1 binds to GSK-3β and is required for β-catenin signaling (Ossipova et al. (2003) Nat Cell Biol 5(10): 889-94).

Fizzled protein signaling is blocked by pertussis toxin (Malbon et al. (2001) Biochem Biophys Res Commun 287(3) as well as sFRPs (secreted Frizzled-related proteins) family, which are similar to frizzled receptors except lacking the TM domains. Other inhibitors of wnt-mediated signaling include collagen 18(XVI11) [Rhen M and Pihlajaniemi T. J Biol Chem 270(9): 4705-11, 1995], endostatin [Hanai J et al, J Cell Biol 156(3): 529-39, 2002], carboxypeptidase Z [Song L and Pricker L D, J Biol Chem 272(16): 10543-50, 1997], receptor tyrosine kinase [Xu Y K, Nusse R. Curr Biol 8(12): R405-6, 1998, Masiakowski, P and Yancopoulos G D, 8(12): R407, 1998], and transmembrane enzyme Corin [Yen W et al, J Biol Chem 274(21): 14926-35, 1999]. When these proteins bind with the wnt proteins, wnt/beta-catenin signaling is suppressed. In addition, other extracellular inhibitors of wnt signaling include, for example WIF-1 (Hsieh J C et al, Nature 398(6726): 431-6, 1999), Cerberus (Picolo et al, Nature 397:707-10), Dickkopf-1 (Tian et al, N Engl J Med: 349(26): 2483-94, 2003) etc. Wise is yet another secreted Wnt inhibitor that binds to LRP, but depending on context can either augment or inhibit Wnt signaling (Itasaki et al. (2003) Development 130(18): 4295 305).

β-catenin is a pivotal player in the wnt/β-catenin signaling pathway, and is controlled by a large number of binding partners that affect its stability and localization. When β-catenin is stabilized and translocates to the nucleus and binds to TCF, (β-catenin displaces a transcriptional repressor bound to TCF called Groucho (grg)), enabling TCF-mediated transcription. Legless and pygopos (Bcl9) also are involved in this complex (Thompson et al. (2002) Nat Cell Biol 4(5): 367-73; Kramps et al. (2002) Cell 109(1): 47-60) and Reptin 52 is also necessary for β-catenin activity.

The accumulation of β-catenin in the cytosol is determined by its interaction with a number of proteins including those in a multiprotein complex of Axin, GSK-3β, APC and other proteins. Axin and APC act as negative regulators of β-catenin, as in the absence of APC, β-catenin is stabilized and goes to the nucleus (Rosin-Arbesfeld et al. (2000) Nature 406(6799): 1009-12; Henderson. (2000) Nat Cell Biol 2(9): 653-60). Chibby is a nuclear antagonist of β-catenin (Takemaru et al. (2003) Nature 422(6934): 905-9) as is pontin 52 (Bauer et al. (2000) EMBO J 19(22): 6121-30).

β-catenin also interacts with Pitx2 (a the transcription factor) (Kioussi et al. (2002) Cell 111(5): 673-85). β-catenin may also be regulated by HMG box factors, such as XSox17 (Zorn et al. (1999) Mol Cell 4(4): 487-98) and HBP1, which functions as a co-repressor of TCF (Sampson et al. (2001) EMBO J. 20(16): 4500-11), although TCF is inhibition by HBP1 is relieved by inhibition of p38 (Xiu et al. (2003) Mol Cell Biol 23(23): 8890-901).

TCF is negatively regulated by phosphorylation by Nemo/NLK kinases, which are stimulated by TAB1/TAK1 kineses (Rocheleau et al. (1999) Cell 97(6): 717-26; Meneghini et al. (1999) Nature 399(6738): 793-7; Ishitani et al. (1999) Nature 399(6738): 798-802; Ishitani, et al. (2003) Mol Cell Biol 23(1): 131 9).

### IV. Method for the Inducing Progenitors to Enter the Isl1⁺ Lineage by Inhibition of Wnt Signaling

In one embodiment, the present invention provides methods to induce uncommitted progenitor cells to enter the Islet 1 lineage pathway and become isl1⁺ progenitors, for example isl1⁺ cardiovascular progenitors, by inhibiting wnt signaling and/or inhibiting or suppressing the wnt/β-catenin pathway.

In one embodiment, methods are provided for the induction of progenitors to enter the islet 1 lineage pathway to form isl1⁺ progenitors by inhibiting the wnt/β-catenin pathway. In some embodiments, one or more agents are used to inhibit or suppress the wnt pathway, herein termed “wnt inhibitory agents” or “inhibitory agents”. In some embodiments wnt inhibitory agents inhibit wnt or homologues thereof, for example wnt3, and in other embodiments, wnt inhibitory agents inhibit components of the wnt/β-canetin-GSK3 pathway, for example but not limited to WLS and DKK1.

Wnt inhibitory agents of the present invention include, but are not limited to, polynucleotides, polypeptides, proteins, peptides, antibodies, small molecules, aptamers, nucleic acids, nucleic acid analogues and other compositions that are capable of selectively inhibiting or suppressing the wnt/β-catenin pathway, or reducing the activity and/or expression of wnt, wnt-dependent genes/proteins and/or β-catenin.

In some embodiments, wnt inhibitory agents useful in the methods of the present invention inhibit and/or suppress the activity of wnt, for example wnt3a. Examples of such wnt inhibitory agents include, but are not limited to, agents that reduce the expression and/or activity of wnt and/or components of the wnt/β-catenin pathway, or induce the expression of repressors and/or suppressors of wnt and/or wnt/β-catenin.

In some embodiments, wnt inhibitor agents directly suppress the expression and/or activity of wnt genes and/or gene products and homologues thereof. Wnt genes include, for example, but are not limited to, Wnt-1, 2A, 2B, 3, 3A, 4, 5A, 5B, 7A, 7B, 8A, 8B, 9A, 9B, 10A, 10B, 11A, and murine Wnt genes, Wnt-1, 2, 3A, 3B, 4, 5A, 5B, 6, 7A, 7B, 8A, 8B, 10B, 11 and 12, the gene or nucleic acid sequences encoding the polypeptides are disclosed in U.S. Pat. Nos. 5,851,984 and 6,159,462, which are incorporated herein by reference in their entirety. In some embodiments, the wnt inhibiting agent comprises an antisense nucleic acid, antisense oligonucleotide, RNAi or other inhibitory molecules directed to one or more or the wnt genes and/or gene products as mentioned above.

In some embodiments, wnt inhibiting agent is a inhibitory nucleic acid, for example an antisense nucleic acid, antisense oligonucleotide (ASO), RNAi, inhibitory or neutralizing antibodies or other inhibitory molecules directed to Wnt3A gene and/or Wnt3A gene product or a modified version, homologue or fragment thereof, for example, but not limited to SEQ ID NO:4 (GenBank accession #NM_{—}009522), SEQ ID NO:5 (GenBank accession #NM_{—}030753); and/or SEQ ID NO:6 (GenBank accession #NM_{—}033131).

In some embodiments, wnt inhibitory agents suppressor are inhibitors and/or inhibitory nucleic acids of essential components of the wnt/β-catenin pathway. Examples include antisense nucleic acids, antisense oligonucleotides (ASO), RNAi, inhibitory or neutralizing antibodies or other inhibitory molecules directed to suppress the Wls/Evi gene or Wls/Evi gene products or homologues thereof. Examples of such a wnt inhibitory agent includes siRNA molecules siWLS-A (SEQ ID NO:1) and siWLS-B (SEQ ID NO:2) as described in the Examples. In alternative embodiments, wnt inhibitory agents can inhibit or be inhibitory nucleic acids to wnt receptors, for example Frizzled receptors and homologues thereof, and alternatively inhibit other essential components of the wnt/β-catenin signaling, including, but not limited to, Dsh (disheveled) LRP-5, LRP-6, Dally (division abnormally delayed), Dally-like, PAR1, β-catenin, TCF, lef-1 and Frodo.

In some embodiments, wnt inhibitory agents can be endogenous suppressors or activate the expression and/or activity of endogenous suppressors of wnt and/or wnt/β-catenin signaling. Such wnt inhibitory agents target endogenous suppressors including, but not limited to, sFRP (secreted frizzled-related proteins), sRFP-1, sFRP-2, collagen 18 (XVIII), endostatin, carboxypeptidase Z, receptor tyrosine kinase, corin, or genetically modified versions, homologues and fragments thereof.

In alternative embodiments, wnt inhibitory agents can be extracellular inhibitors of wnt signaling including, but not limited to, WIF-1, cerberus, Dickkopf-1 (DKK1), Dapper, pertussis toxin, disabled-2 (dab-2), naked cuticle (naked), Frzb-related proteins, FrzA, frzB, sizzled and LRP lacking the intracellular domain and generically modified versions, homologues and fragments thereof. In one embodiment, wnt inhibitory agents that potentiate or enhance sFRP expression are encompassed for use in the present invention, for example expression of Dgl gene, as discussed in European Patent Application No. EPO 1,733,739, which is incorporated herein by reference in its entirety.

In further aspects, wnt inhibitory agent can inhibit β-catenin, for example, by reducing and/or inhibiting the accumulation of β-catenin in the cytoplasm and/or promoting phosphorylation of β-catenin. In such embodiments, wnt inhibitory agents that inhibit β-catenin include, but are not limited to, protein phosphatase 2 (PP2A), chibby, pontin 52, Nemo/LNK kinase, and HMG homobox factors, for example, XSox17, HBP1, APC, Axin, disabled-2 (dab-2), and grucho (grg).

Alternatively, wnt inhibitory agents useful in the present invention can be agents capable of increasing the activity and/or expression of genes and/or protein that suppress the activity and/or expression of wnt or the wnt/β-catenin pathway including, but not limited to, agents that activate or enhance the activity GSK-3 and/or GSK-3β. For example, wnt inhibitory agents can activate or increase the expression of suppressors of wnt and/or wnt/β-catenin signaling. An example of such an embodiment is activation of GSK-3, for example, wnt inhibitory agents can be agents which dephosphorylate (activate) GSK-3. The GSK-3β polypeptide sequences include, but are not limited to, SEQ ID NO:7 (GenBank accession #NM_{—}002093). In alternative embodiments, the wnt inhibitory agents useful in the present invention that activate GSK3 and/or GSK3β are, for example, agents that trigger PKB-mediated signalling, for example wortannin.

It is encompassed in the present invention that wnt inhibitory agents prevent the wnt/β-catenin signaling in the progenitor cell that is to be induced to enter the islet 1 lineage pathway. For example, wnt inhibitor agents can be delivered to culture media of a progenitor cell, and in some embodiments the wnt inhibitory agent is delivered to the progenitor cell as a polynucleotide and/or a polypeptide. The polynucleotide can be comprised in a vector, (i.e., a viral vector and/or non-viral vector). For example, viral vectors can include adenoviral vectors, adeno-associated viral (AAV) vectors, retroviral vectors or a lentiviral vector. Alternatively, the wnt inhibitory agent may be delivered to a feeder layer, for example a cardiac mesenchymal cell (CMC) feeder layer, such that the wnt/β-catenin signaling is inhibited at the level of the feeder layer. In some embodiments, the feeder layer may comprise ‘wnt inhibitory agent-producing cells’. In alternative embodiments, wnt inhibitory agents are delivered to the progenitor cell and/or the feeder layer. In some embodiments, more than one wnt inhibitory agent is delivered to the progenitor cells and/or feeder layer, and in some embodiments, the wnt inhibitory agents delivered to the progenitor cell are different from those delivered to the feeder layer. In some embodiments, the expression of a nucleic acid encoding a wnt inhibitory agent is operatively linked to a promoter, and in some embodiments, the promoter is an inducible promoter.

### V. Method for the Renewal and Proliferation of Isl1+ Progenitor by Activation of Wnt Signaling

Another aspect of the present invention provides methods for the renewal and expansion of isl1⁺ progenitor cells, for example isl1⁺ cardiovascular progenitor by activating wnt/β-catenin signaling. In some embodiments, the methods provide renewal of isl1⁺ progenitor by activating wnt/β-catenin signaling in a feeder-free system, and in alternative embodiments, the methods provide renewal of isl1⁺ progenitor by activating wnt/β-catenin signaling in the presence of a feeder-layer, for example a cardiac mesenchymal cell (CMC) feeder layer. Therefore, the present invention provides methods to enhance renewal of isl1⁺ in the presence or absence of a feeder cell layer.

Accordingly, in some embodiments of the present invention, methods for the expansion and renewal of isl1⁺ progenitors by activating the wnt/β-catenin pathway are provided. In some embodiments, one or more agents are used to activate or enhance the wnt pathway, herein termed “wnt activating agents” or “activating agents”. In some embodiments wnt activating agents activate the wnt/β-catenin pathway directly, for example wnt activating agents include wnt or wnt3a or homologues and variants thereof, as well as β-catenin and components of the wnt/β-catenin signaling pathway. In other embodiments, wnt activating agents activate wnt/β-catenin pathway by inhibiting negatively acting components of the wnt/β-canetin-GSK3 pathway. For example, a wnt activating agent can suppress or inhibit the activity and/or expression of wnt/β-catenin endogenous suppressors, for example a wnt activating agent can be an inhibitor of GSK3β.

Wnt activating agents of the present invention include, but are not limited to polynucleotides, polypeptides, proteins, peptides, antibodies, small molecules, aptamers, nucleic acids, nucleic acid analogues and other compositions that are capable of activating or enhancing the wnt/β-catenin pathway, or increasing the activity and/or expression of wnt, wnt-dependent genes/proteins and/or β-catenin. Alternatively, wnt activating agents of the present invention are agents that inhibit the activity and/or expression of genes and/or gene products that suppress the activity and/or expression of wnt or the wnt/β-catenin pathway including, but not limited to, agents that inhibit GSK-3 or GSK-3β, or sFRP, DKK1, WIF-1 etc.

In one embodiment, wnt activating agents activate and/or increase the activity of wnt homologues and/or wnt/β-catenin signaling. In some embodiments, wnt activating agents are a wnt gene and/or wnt gene product, or homologues or genetically modified versions and fragments thereof having wnt signaling activity. Wnt genes and proteins useful as wnt activating agents in the present invention are well known to a person of ordinary skill in the art, and include, for example, human and mouse wnt genes, wnt homologues and fragments and genetically modified versions thereof that have wnt signaling activity. Wnt genes include, but are not limited to human Wnt-1, 2A, 2B, 3, 3A, 4, 5A, 5B, 7A, 7B, 8A, 8B, 9A, 9B, 10A, 10B, 11A, and murine Wnt genes, Wnt-1, 2, 3A, 3B, 4, 5A, 5B, 6, 7A, 7B, 8A, 8B, 10B, 11 and 12. Gene or nucleic acid sequences encoding the polypeptides are disclosed in U.S. Pat. Nos. 5,851,984 and 6,159,462, which are incorporated herein by reference in their entirety. In some embodiments, the wnt activating agent comprises one or more wnt gene and/or gene product as mentioned above. In some embodiments, the wnt activating agent is Wnt3A gene or Wnt3A gene product or a modified version, homologue or fragment thereof, that has wnt signaling activity, including, but not limited to SEQ ID NO:4 (GenBank accession #NM_{—}009522), SEQ ID NO:5 (GenBank accession #NM_{—}030753); and/or SEQ ID NO:6 (GenBank accession #NM_{—}033131). Other wnt activating agents that activate wnt/β-catenin signaling can be used, for example compositions listed and discussed in U.S. Pat. Nos. 5,851,984 and 6,159,462 which are incorporated herein by reference in their entirety.

In alternative embodiments, wnt activating agents include but are not limited to disheveled WLS/Evi, (dsh), LRP-5, LRP-6, Dally (division abnormally delayed), Dally-like, PAR1, β-catenin, TCF, lef-1 and Frodo or homologues or genetically modified versions thereof that retain wnt activating activity. In some embodiments, wnt activating agents are inhibitory molecules to endogenous extracellular inhibitors of wnt/β-catenin signalling, for example inhibitors that inhibit their activity and/or expression, for example inhibitory nucleic acid of WIF-1, cerberus, Dickkopf-1 (DKK1), Dapper, pertussis toxin, disabled-2 (dab-2), naked cuticle (naked), Frzb-related proteins, FrzA, frzB, sizzled sFRP (secreted frizzled-related proteins), sRFP-1, sFRP-2, collagen 18 (XVIII), endostatin, carboxypeptidase Z, receptor tyrosine kinase, corin etc.

In further aspects, wnt activating agents trigger wnt/β-catenin signaling by activating and/or increasing the activity of β-catenin, for example, that stabilize and/or increase cytosolic accumulation of β-catenin and/or inhibit its phosphorylation. In some embodiments, wnt activating agents are β-catenin gene and/or β-catenin gene product, or homologues, genetically modified version or fragments thereof that retain wnt activating activity. β-catenin gene and gene product are known to persons of ordinary skill in the art, and include but are not limited to SEQ ID NO:7 (GenBank accession #XM_{—}208760). In some embodiments, wnt activating agents are stabilized versions of β-catenin, for example versions where serine residues of the GSK-3β phosphorylation consensus motif of β-catenin have been substituted, resulting in inhibition of ubiquitination and stabilization of the protein. Examples of stabilized β-catenins include, but are not limited to those with the amino acid changes D32Y; D32G; S33F; S33Y; G34E; S37C; S37F; T41I; S45Y; and deletion of AA 1-173 relative to human β-catenin. A number of publications describe stabilized β-catenin mutations, for example, see Morin et al., 1997; Palacios et al., 1998; Muller et al., 1998; Miyoshi et al., 1998; Zurawel et al., 1998; Voeller et al., 1998; and U.S. Pat. No. 6,465,249, etc., which are incorporated herein in their entirety by reference. In alternative embodiments, other wnt activating agents that activate β-catenin can be used, for example compositions discussed in U.S. Pat. No. 6,465,249, which is incorporated herein in its entirety by reference.

In alternative embodiments, wnt activating agents are any β-catenin binding partners that increase the stability of β-catenin and/or promote β-catenin localization in the nucleus. In alternative embodiments, wnt activating agents include, but are not limited to Frodo, TCF, pitx2, Reptin 52, legless (lgs), pygopus (pygo), hyrax/parafbromin, LKBI/XEEK1 or homologues or modified versions or fragments thereof that retain wnt activating activity. In alternative embodiments, wnt activating agents are inhibitors of negative factors, for example inhibitory nucleic acids and/or peptides that inhibit the activity and/or gene expression of, for example but not limited to APC, Axin, dab-2, grucho, PP2A, chibby, pontin 52, Nemo/LNK kinases etc.

In another embodiment, wnt activating agents useful in the present invention are inhibitors of GSK-3 and/or GSK-3β. Examples of inhibitors of GSK-3 inhibitors include but are not limited to BIO (6-bromoindirubin-3′ oxime), acetoxime analogue of BIO, 1-azakenpaullone or analogues or modified versions thereof, as shown in the Examples. In some embodiments, wnt activating agents can be substrate competitive GSK3 peptides, for example the cell permeable substrate competitive GSK3 peptide (SEQ ID NO:3) as discussed in the Examples. Any agent which inhibits GSK3β is potentially useful as a wnt activating agent in the methods described herein, and includes, for example lithium, LiCl, Ro31-8220, as disclosed in International Patent Application No: PCT97/41854, which is incorporated herein in its entirety by reference, and retinoic acid.

In alternative embodiments, other wnt activating agents that inhibit GSK-3 can be used, for example compositions disclosed in U.S. Pat. No. 6,411,053, which is incorporated herein by reference in its entirety. The present invention also encompasses all GSK-3 inhibitors, including those discovered as GSK-3 inhibitors by the methods disclosed in International Patent Application No: PCT97/41854, which is incorporated herein in its entirety by reference.

It is encompassed in the present invention that wnt activating agents activate or enhance Wnt/β-catenin signaling in the isl1⁺ progenitor to be renewed. For example, wnt activating agents can be delivered to the culture media of the isl1⁺ progenitor, and in some embodiments the wnt activating agent is delivered to the isl1⁺ progenitor as a polynucleotide and/or a polypeptide. The polynucleotide can be comprised in a vector, (i.e., a viral vector and/or non-viral vector). Examples of the viral vectors include, but are not limited to adenoviral vectors, adeno-associated vectors, retroviral vectors or lentiviral vectors. Alternatively, wnt activating agents may be delivered to a feeder layer, for example a cardiac mesenchymal cell (CMC) feeder layer, such that the wnt/β-catenin signalling is promoted in the feeder layer. In one embodiment, the feeder layer may comprise ‘wnt activating agent-producing cells’. In alternative embodiments, wnt activating agents are delivered to the isl1⁺ progenitor and/or the feeder layer. In some embodiments, more than one wnt activating agent is delivered to the isl1⁺ progenitor cell and/or feeder layer, and in some embodiments, the wnt activating agents delivered to the isl1⁺ progenitor cell are different from those delivered to the feeder cell layer. In some embodiments, the wnt activating agent can be encoded in a nucleic acid operatively linked to a promoter, and in some embodiments the promoter is, for example, a tissue-specific promoter, or an inducible promoter, or regulated by isl1⁺ expression.

### VI. Cells

In one aspect of the invention, methods to trigger a cell to enter the islet 1 lineage pathway are provided. In such an embodiment, the methods of the present invention comprise inhibiting wnt and/or wnt/β-catenin signalling, such that a cell, for example a uncommitted progenitor, enters the islet 1 lineage pathway to become an isl1⁺ progenitor.

In some embodiments, the cell that is induced to enter the islet 1 lineage pathway to become an isl1⁺ progenitor is a stem cell or a progenitor, for example an uncommitted progenitor. In some embodiments, the progenitor is a mesoderm progenitor. In some embodiments, the progenitor is a human progenitor.

In some embodiments, the cell that is induced to enter the islet 1 lineage pathway to become an isl1⁺ progenitor is a human cell, and in some instances the cell is a human stem cell, for example a human ES cell, as shown, for example, in Example 6.

In one aspect, the progenitor cells for use to be induced to enter the islet 1 lineage pathway to become isl1⁺ progenitors can be a cell derived from any kind of tissue, for example embryonic tissue such as fetal or pre-fetal tissue, neonatal or adult tissue.

In an important embodiment, the tissue is from a human. In some embodiments, the tissue is from a mammal, for example a mouse and in some embodiments the tissue us from a genetically modified mouse, for example a transgenic mouse.

In some embodiments, the cell to be induced to enter the islet 1 lineage pathway to become isl1⁺ progenitors is obtained from tissue including solid tissues (the exception to solid tissue is whole blood, including blood, plasma and bone marrow) which were previously unidentified in the literature as including progenitor or stem cells are also within the scope of this invention. In some embodiments, the tissue is heart or cardiac tissue. In other embodiments, the tissue includes but is not limited to umbilical cord blood, placenta, bone marrow, and chondral villi. In some embodiments, the progenitors are derived from tissue obtained from a subject with a disease or disorder. As an exemplary embodiment, the tissue is cardiac tissue and the cardiac tissue is obtained from a subject with a cardiac disorder or coronary disease, for example an acquired and/or congenital cardiac disorder or coronary disorder. In some embodiments, the tissue is obtained from a biopsy of tissue from a subject with a disease or disorder, for example a subject having an acquired and/or congenital cardiac disorder or coronary disorder.

In some embodiments, the cell for use to be induced to enter the islet 1 lineage pathway to become isl1⁺ progenitors are genetically modified. In some embodiments, the cell can be genetically modified to comprise, for example, nucleic acids encoding reporter genes for identification of cells differentiated along specific lineages. In another embodiment, the cell can be genetically modified to either correct a pathological characteristic, for example a disease and/or genetic characteristic associated with a disease or disorder. In some embodiments the disease or disorder is a cardiovascular disease or disorder. In some embodiments, the cell can be genetically modified to comprise a characteristic associated with a disease or genetic defect, for example, such cell can be useful in studying the pathology of a disease. In some embodiments, the cells are genetically engineered to comprise a wnt inhibiting agent. Such methods to genetically engineer the cells useful to be induced to enter the islet 1 lineage pathway to become isl1⁺ progenitors are well known by those skilled in the art, and include introducing nucleic acid into the cells by means of transfection, for example but not limited to the use of viral vectors or by other means known in the art.

In some embodiments, the progenitor is any cell having a characteristic of being capable, under appropriate conditions, of producing progeny of different cell types that are derivatives of all of the 3 germinal layers (endoderm, mesoderm, and ectoderm). In some embodiments, such cells are cell types provided in the form of an established cell line, or they may be obtained directly from primary embryonic tissue and used immediately for the methods of the present invention to induce their entry to islet 1 lineage pathway. Included are cells listed in the NIH Human Embryonic Stem Cell Registry, e.g. hESBGN-01, hESBGN-02, hESBGN-03, hESBGN-04 (Bresagen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

Progenitors for use in the aspect of the present invention related to methods to induce their entry towards islet 1 lineages include also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.). In particular embodiments, the stem cells may be obtained from any mammalian species, e.g. human, equine, bovine, porcine, canine, feline, rodent, e.g. mice, rats, hamster, primate, etc.

ES cells are considered to be undifferentiated when they have not committed to a specific differentiation lineage. Such cells display morphological or phenotype characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid Gb5, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of cardiac, endothelial, muscle, and/or neural stem cells, as described above, is harvested from a mammalian donor by methods known in the art. A suitable source is the hematopoietic microenvironment. For example, circulating peripheral blood, preferably mobilized (i.e., recruited) as described below, may be removed from a subject. Alternatively, bone marrow may be obtained from a mammal, such as a human patient, undergoing an autologous transplant.

Human umbilical cord blood cells (HUCBC) are useful for the methods of the present invention as a source of cell be induced to enter the islet 1 lineage pathway to become isl1⁺ progenitors. HUCB cells have recently been recognized as a rich source of hematopoietic and mesenchymal progenitor cells (Broxmeyer et al., 1992 Proc. Natl. Acad. Sci. USA 89:4109-4113).

Progenitors useful in the methods of the present invention can be obtained from placenta, amniotic fluid, and choronic villi, as cited in International Patent Application: WO/03042405, which is incorporated in its entirety herein by reference.

One source of cells useful for the methods of the present invention as a source of cells to be induced to enter the islet 1 lineage pathway to become isl1⁺ progenitors are cells from a hematopoietic micro-environment, such as the circulating peripheral blood, preferably from the mononuclear fraction of peripheral blood, umbilical cord blood, bone marrow, umbilical fluid, fetal liver, or yolk sac of a mammal. The stem cells, especially neural stem cells, may also be derived from the central nervous system, including the meninges.

In some embodiments, the present invention provides methods to expand isl1⁺ progenitors by triggering their renewal by activating and/or enhancing wnt/β-catenin signaling. In such embodiments, the isl1⁺ progenitors for expansion by the methods of the present invention are isl1⁺ progenitors produced by the inducing a cell to enter the islet 1 lineage by the methods provided herein. In alternative embodiments, the isl1⁺ progenitors are obtained by other means known by persons of ordinary skill in the art. In some embodiments, the isl1⁺ progenitors are isolated by the methods described by Provisional Patent Application No. 60/856,490 which is incorporated herein in its entirety by reference.

The methods described herein provide expansion of isl1⁺ progenitors in the presence or absence of a cell feeder layer, for example a cardiac mesenchymal cell (CMC) feeder cell layer, by activating or enhancing wnt/β-catenin signaling using wnt activating agents. The isl1⁺ progenitors can be also induced to differentiate and/or mature in the presence or absence of the feeder cell layer by addition of factors to induce differentiation, by such methods that are commonly known in the art. Such factors are also referred to as differentiating agents. Differentiating agents can be, for example, any growth factors or differentiation-inducing factor which induces the isl1⁺ progenitor to differentiate along specified lineages. Differentiating agents can be added to the medium, as well as to a supporting structure (such as a substrate on a solid surface) to induce differentiation. Differentiation may be initiated by allowing the stem cells to form aggregates, or similar structures; for example, aggregates can result from overgrowth of a stem cell culture, or by culturing the stem cells in culture vessels having a substrate with low adhesion properties.

In one embodiment, embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after about 7 days to about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the progenitors can be de-differentiated or retrodifferentiated progenitors, such as progenitors derived from differentiated cells. In such an embodiment, the de-differentiated stem cells can be, for example, cardiac cells, neoplastic cells, tumor cells, cancer cells and cancer stem cells. Such an embodiment is useful in identifying and/or isolating and/or studying cancerous cells and tumor cells. In some embodiments, the de-differentiated cells are from a subject, and in some embodiments, the de-differentiated stem cells are obtained from a biopsy.

### VII. Agents

One aspect of the invention relates to use of wnt inhibitory agents and wnt activating agents. Example of wnt activating- and wnt-inhibiting agents can include for example nucleic acids, peptides, nucleic acid analogues, phage, phagemids, polypeptides, peptidomimetics, antibodies, small or large organic molecules, ribozymes or inorganic molecules or any combination of the above. Wnt inhibitory agents and wnt activating agents can also be naturally occurring or non-naturally occurring (e.g., recombinant) and are sometimes isolated and/or purified.

In some embodiments, the wnt inhibitory agents and wnt activating agents include for example antibodies (polyclonal or monoclonal), neutralizing antibodies, antigen-binding antibody fragments, peptides, proteins, peptide-mimetics, aptamers, oligonucleotides, hormones, small molecules, nucleic acids, nucleic acid analogues, carbohydrates or variants thereof that function to inactivate or activate one or more, as the case may be, nucleic acid and/or protein participant in a wnt pathway as described herein or as known in the art. Nucleic acids include, but are not limited to DNA, RNA, oligonucleotides, peptide nucleic acid (PNA), pseudo-complementary-PNA (pcPNA), locked nucleic acid (LNA), RNAi, microRNAi, siRNA, shRNA etc. A nucleic acid may be single or double stranded, and can be selected from a group comprising; nucleic acid encoding a protein of interest, oligonucleotides, PNA, etc. Such nucleic acid sequences include, but are not limited to nucleic acid sequence encoding proteins that act as transcriptional repressors, antisense molecules, ribozymes, small inhibitory nucleic acid sequences (including, but not limited to RNAi, shRNAi, siRNA, micro RNAi (mRNAi)) and antisense oligonucleotides, etc. A protein and/or peptide inhibitor or fragment thereof, can include, but is not limited to mutated proteins; therapeutic proteins and recombinant proteins. Proteins and peptide inhibitors can also include, for example, genetically modified proteins and peptides, synthetic peptides, chimeric proteins, antibodies, humanized proteins, humanized antibodies, chimeric antibodies, monoclonal and polyclonal antibodies, modified proteins and wnt pathway-activating or inhibiting fragments thereof.

Agent used herein as wnt inhibitory agents and/or wnt activating agents can be also selected from a group comprising chemical, small molecule, chemical entity, nucleic acid sequences, nucleic acid analogues or protein or polypeptide or analogue of fragment thereof.

The agent may be applied to the media, where it contacts the cell (such as the progenitor and/or feeder cells) and induces its effects. Alternatively, the agent may be intracellular within the cell (for example intracellular within a progenitor and/or feeder cells) as a result of introduction of the nucleic acid sequence into the cell and its transcription resulting in the production of the nucleic acid and/or protein agent within the cell. An agent also encompasses any action and/or event the cells are subjected to. As non-limiting examples, an action can comprise any action that triggers a physiological change in the cell, including, for example heat-shock, ionizing irradiation, cold-shock, electrical impulse, light and/or wavelength exposure, UV exposure, pressure, stretching action, increased and/or decreased oxygen exposure, exposure to reactive oxygen species (ROS), ischemic conditions, fluorescence exposure etc. The exposure to agent may be continuous or non-continuous, and in some embodiments, cells may be exposed to wnt inhibitory agents and wnt activating agents in alternating exposures.

In further embodiments of the invention, modified versions of wnt inhibitory agents and wnt activating agents are encompassed. For instance, wnt inhibitory and/or activating agents may also be fusion proteins from one or more proteins, chimeric proteins (for example domain switching or homologous recombination of functionally significant regions of related or different molecules), synthetic proteins or other protein variations including substitutions, deletions, insertion and other variants.

It will be appreciated by those of skill that the genes identified herein and those identified by the methods of the present invention can be readily manipulated to alter the amino acid sequence of a protein. Genes including, but not limited to wnt, β-catenin, GSK-3β, WLS, sFRP etc and wnt-pathway active homologues or variants thereof can be manipulated by a variety of well known techniques for in vitro mutagenesis, among others, to produce variants of the naturally occurring human protein or fragment thereof, herein referred to as muteins, which may be used in accordance with the invention.

Similarly, techniques for making small oligopeptides and polypeptides that function as wnt activating agents or wnt inhibitory agents as dominant negative versions (i.e inactive versions) of larger proteins from which they are derived are known in the art. Thus, peptide analogs of genes and gene products of the invention that inactivate the gene product also are useful in the invention.

In some embodiments, RNA interference or “RNAi” can be used as wnt inhibitory agents and wnt activating agents. In such embodiments, an RNAi molecule that negatively regulates the expression of the gene products, for example but not limited to WLS, wnt, GSK-3β, β-catenin, etc. can be used.

In another embodiment, suitable wnt inhibitory agents and wnt activating agents may be achieved by introducing catalytic antisense nucleic acid constructs, such as ribozymes, which are capable of cleaving RNA transcripts and thereby preventing the production of wildtype protein. Ribozymes are targeted to and anneal with a particular sequence by virtue of two regions of sequence complementary to the target, flanking the ribozyme catalytic site. After binding the ribozyme cleaves the target in a site specific manner. The design and testing of ribozymes which specifically recognize and cleave sequences of the specific gene products is known to persons of ordinary skill in the art.

In some embodiments, suppression of wnt/β-catenin signaling by wnt inhibitory agents or activation of wnt/β-catenin by wnt activating agents may be performed by addition of agents, for example wnt inhibitory agents or wnt activating agents to a cell culture medium.

In alternative embodiments, suppression of wnt/β-catenin signaling by wnt inhibitory agents or activation of wnt/β-catenin by wnt activating agents may be performed by mixing a cell culture medium with a cell culture medium of a wnt inhibitory agent-producing cell or a wnt activating agent-producing cell. An “agent-producing cell” as defined herein, refers to any cell that secretes or results in an increase in the extracellular amount of an agent, for example a wnt activating- or wnt inhibitory agent. An example of an agent-producing cell is a cell secreting the wnt activating agent Wnt3A, as discussed in Example 3, which teaches use of wnt3a conditioned media harvested from wnt3a secreting feeder cells. In alternative embodiments, a cell can comprise wnt inhibitory agents or wnt activating agents, and herein is referred to as an “agent-comprising cell”, where the cell comprises a wnt inhibitory agent or wnt activating agent that functions intrinsic within that cell. For example, the cell may comprise an inhibitory nucleic acid WLS as discussed in Example 4 that suppresses the wnt/β-catenin by suppressing release of wnt from the cell. In some embodiments, the agent-producing cell or agent comprising cell may be transfected with a gene encoding a wnt inhibitory agent or wnt activating agent. In some embodiments, the agent-producing cell or agent comprising cell is comprised by or consists of a feeder cell layer. And in some embodiments, the agent-producing or agent comprising feeder cell layer is an agent-producing cardiac mesenchymal cell (CMC) feeder layer. For example, the CMC feeder layer can be a wnt inhibitory agent-producing CMC useful in inducing progenitors to enter the islet 1 lineage, or alternatively, the CMC feeder layer can be a wnt activating agent-producing CMC feeder layer useful in promoting renewal of isl1⁺ progenitors.

Suppression or inhibition of wnt/β-catenin signaling may also be performed by genetically modifying the progenitor cell to be induced to enter the islet 1 lineage. Alternatively, activation or enhancing wnt/β-catenin signalling may also be performed by genetically modifying the isl1⁺ progenitor to be renewed.

In alternative embodiments, suppression of wnt/β-catenin signaling by wnt inhibitory agents or activation of wnt/β-catenin by wnt activating agents may be performed by coating wnt inhibitory agents or wnt activating agents on a cell culture plate, a three-dimensional cell culture bead, a culture support or combinations thereof.

In some embodiments, wnt inhibitory agents and wnt activating may be administered to the cell, for example the progenitor cell, or feeder layer cell in a vector. The vector may be a plasmid vector, a viral vector, or any other suitable vehicle adapted for the insertion of foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, retroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the agonist or antagonist nucleic acid molecule in the subject in high copy number extra chromosomal DNA thereby eliminating potential effects of chromosomal integration.

In some embodiments, a cell may be genetically manipulated to comprise a vector encoding a wnt activating agent and a wnt inhibitory agent. In such an embodiment, the nucleic acid encoding a wnt inhibitory agent is operatively linked to a promoter and the nucleic acid encoding a wnt activating agent is operatively linked to a different promoter. In some embodiments, the promoters are tissue specific promoters, and in alternative embodiments, the promoters are inducible promoters. In some embodiments the inducible promoters are induced by opposite signals. As an non-limiting example, a vector could comprise a wnt inhibitory agent operatively linked to a inducible promoter, for example a “tet on” promoter, and the wnt activating agent is operatively linked to an oppositely (i.e. antagonistically) regulated inducible promoter, for example a “tet-off” promoter. In such embodiments, in the presence of tet or an analogue thereof, the wnt inhibitory agent is expressed and the cell is induced to enter the islet 1 lineage, and in the absence of tet or an analogue thereof, the wnt activating agent is expressed in the cell (and expression of the wnt inhibitory agent is shut off) and the cell is triggered to renew. In some embodiments, the wnt activating agent can be operatively linked to a promoter that is regulated by the expression of islet 1. In another embodiment, the wnt activating agent can be operatively linked to a promoter that is activated by the expression of islet 1⁺ or other markers expressed in isl1⁺ progenitors, for example markers expressed in isl1⁺ cardiovascular progenitors, for example transcription factor markers expressed in isl1⁺ cardiovascular progenitors, such as, but not limited to Nkx2.5, flk-1, Mef2-C, GATA-4 and other markers commonly known by persons skilled in the art.

### VIII. Methods to Identify a Cell which is of Isl¹⁺ Lineage

In some embodiments, one can determine if a cell has entered the Isl1+ lineage by identifying if the cell expresses an Isl1⁺ RNA transcript or protein. As disclosed herein, a cell which has entered the Isl1⁺ lineage is a Isl1⁺ progenitor cell which capable of differentiating into multiple different lineages. An Isl1⁺ progenitor cell which capable of differentiating into multiple different lineages can be identified by contacting the stem cells with agents that are reactive to Islet1+ and isolating the positive cells from the non-reactive cells, where the positive cells are Islet1-positive are Isl1⁺ progenitor cells. In some embodiments, the an Isl1⁺ progenitor cell which capable of differentiating into multiple different lineages can be identified by contacting the stem cells with agents that are reactive to Islet1⁺, Nkx2.5 and flk1 and isolating the positive cells from the non-reactive cells, where the positive cells are Isl1-positive, Nkx2.5-positive and flk1-positive are Isl1⁺ progenitor cells capable of differentiating into multiple different lineages, such as the three main types of lineages which make up the heart; cardiac, smooth muscle and endothelial cells.

In some embodiments, the agents are reactive to nucleic acids and in another embodiment the agents are reactive to the expression products of the nucleic acids encoding one or more of Isl1+, Nkx2.5 and flk1. Another embodiment encompasses isolating the Isl1⁺ progenitor cells expressing Isl1, Nkx2.5 and flk1 using conventional methods of using a marker gene operatively linked to the promoter of Isl1 and/or Nkx2.5 and/or flk1. In some embodiments therefore, one can identify if a cell has entered the Isl1+ lineage by contacting the cell with agents reactive to at least Islet1, and in some embodiment the agent is reactive to Nkx2.5 and flk1, and identifying and separating the reactive positive cells which are the cells that have entered the Isl1+ lineage from non-reactive cells.

Methods to determine the expression, for example the expression of RNA or protein expression of markers of a cell of a Isl1⁺ lineage, such as an Isl1+ progenitor as disclosed herein, such as expression of Isl-1, and optionally the expression of Nkx2.5 and Flk1 expression are well known in the art, and are encompassed for use in this invention. Such methods of measuring gene expression are well known in the art, and are commonly performed on using DNA or RNA collected from a biological sample of the cells, and can be performed by a variety of techniques known in the art, including but not limited to, PCR, RT-PCR, quantitative RT-PCR (qRT-PCR), hybridization with probes, northern blot analysis, in situ hybridization, microarray analysis, RNA protection assay, SAGE or MPSS. In some embodiments, the probes used detect the nucleic acid expression of the marker genes can be nucleic acids (such as DNA or RNA) or nucleic acid analogues, for example peptide-nucleic acid (PNA), pseudocomplementary PNA (pcPNA), locked nucleic acid (LNA) or analogues or variants thereof.

In other embodiments, the expression of the markers can be detected at the level of protein expression. The detection of the presence of nucleotide gene expression of the markers, or detection of protein expression can be similarity analyzed using well known techniques in the art, for example but not limited to immunoblotting analysis, western blot analysis, immunohistochemical analysis, ELISA, and mass spectrometry. Determining the activity of the markers, and hence the presence of the markers can be also be done, typically by in vitro assays known by a person skilled in the art, for example Northern blot, RNA protection assay, microarray assay etc of downstream signaling pathways of isl1. In particular embodiments, qRT-PCR can be conducted as ordinary qRT-PCR or as multiplex qRT-PCR assay where the assay enables the detection of multiple markers simultaneously, for example isl-1 and Nkx2.5 and/or Flk1, either together or separately from the same reaction sample.

In another embodiment, a cell which has entered the Isl1+ lineage can be identified functionally by its ability to differentiate along multiple lineages. In some embodiments, a cell which has entered the Isl1+ lineage is capable of differentiating into a plurality of subtypes of cardiovascular progenitors, for example but not limited to cardiovascular vascular progenitors and cardiovascular muscle progenitors. In some embodiments, a cell of a Isl1⁺ lineage, such as an Isl1+ progenitor which is also Nkx2.5⁺ and flk1⁺ positive can differentiate along cardiovascular vascular progenitor lineages to produce progeny which are Islet-1-positive, Flk1-positive and Nkx2.5-negative cardiovascular vascular progenitors. Alternatively, a cell of a Isl1⁺ lineage, such as an Isl1+ progenitor which is also Nkx2.5⁺ and flk1⁺ positive can differentiate along cardiovascular muscle progenitor lineages to produce Islet-1-positive, Nkx2.5-positive and Flk1-negative cardiovascular muscle progenitors, or Nkx2.5-positive, Islet-1-negative and Flk1-negative cardiovascular muscle progenitors.

In further embodiments, a cell of an Isl1⁺ lineage, such as an Isl1+ progenitor which is also Nkx2.5⁺ and flk1⁺ positive is capable of differentiating into endothelial lineages, myocyte lineages, neuronal lineages, autonomic nervous system progenitors. For example, a cell of an Isl1⁺ lineage, such as an Isl1+ progenitor which is also Nkx2.5⁺ and flk1⁺ positive which as differentiated along endothelial lineages can be identified by endothelial markers, for example but not limited to cells expressing markers PECAM1, flk1, CD31, VE-cadherin, CD146, vWF and other endothelial markers commonly known by persons of ordinary skill in the art. For example, a cell of an Isl1⁺ lineage, such as an Isl1+ progenitor which is also Nkx2.5⁺ and flk⁺ positive which as differentiated along smooth muscle lineages can be identified by smooth muscle markers, for example but not limited to cells expressing markers smooth muscle actin (SMA or SM-actin) or smooth muscle myosin heavy chain (SM-MHC) and response to vasoactive hormone Angotensin II to result in a progressive cytosolic [Ca2⁺], increase or other smooth muscle markers commonly known by persons of ordinary skill in the art. For example, a cell of an Isl1⁺ lineage, such as an Isl1+ progenitor which is also Nkx2.5⁺ and flk1⁺ positive which as differentiated along cardiomyocyte lineages can be identified by expressing troponin (TnT), TnT1, α-actinin, atrial natruic factor (ANT), acetylcholinesterase and other cardiomyocyte markers commonly known by persons of ordinary skill in the art.

In some embodiments, a cell of an Isl1⁺ lineage, such as an Isl1+ progenitor which is also Nkx2.5⁺ and flk1⁺ positive can be identified by its ability to differentiate into cells having an autonomic nervous system phenotype; cells having a neural stem cell phenotype, cells having a myocytic phenotype, cells having an endothelial phenotype. For example, cells having neural stem cell phenotype express a neural marker, such as Nestin, Neu, NeuN or other neuronal precursor markers, and cells with myocytic phenotype or myocyte phenotype, or cardiomyocyte phenotype markers such as, but not limited to, ANP (Atrial natriuretic peptide), Arpp, BBF-1, BNP (B-type natriuretic peptide), Caveolin-3 (Cav-3), Connexin-43, Desmin, Dystrophin (Xp21), EGFP, Endothelin-1, Fluoromisonidazole, FABP (Heart fatty-acid-binding protein), GATA-4, GATA-5 MEF-2 (MEF2), MLC2v, Myosin, N-cadherin, Nestin, Popdc2 (Popeye domain containing gene 2), Sarcomeric Actin, Troponin or Troponin I.

In some embodiments, a cell of an Isl1⁺ lineage, such as an Isl1+ progenitor can be identified by its ability to differentiate along autonomic nervous system lineage have cardiac autonomic nervous system phenotype, for example express acetylycholinesterase. In some embodiments, a cell of an Isl1⁺ lineage, such as an Isl1+ progenitor can be identified by its ability to differentiate along cardiac autonomic cell type have cardiac pace maker phenotype and/or conduction phenotype, and can be identified by markers such as EGFP (Kolossov et al, FASAB J, 2005; 19; 577-579) or other electrical properties of the cells commonly known by persons of ordinary skill in the art.

In some embodiments, an agent useful in the methods as disclosed herein which are reactive to a protein or expression product (such as RNA), for example a protein or RNA for Isl1 can be, for example, a nucleic acid agent; small molecule; aptamer; protein; polypeptide or fragment or variant thereof, such as, for example, DNA; RNA; PNA; pcPNA; locked nucleic acid (LNA) and analogues thereof. In some embodiments, a nucleic acid agent is selected from a group consisting of; RNA; messenger RNA (mRNA) or genomic DNA. In some embodiments, an agent is reactive to a protein or fragment thereof, for example, such agents include an antibody, aptamer or antibody fragments and the like. In some embodiments, an agent is labeled, for example by a fluorescent label as disclosed herein. In some embodiments, an agent is reactive to the nucleic acid encoding markers of a cell of the Isl1⁺ lineage, or protein of a marker of cell of the Isl1⁺ lineage such as Isl1⁺ progenitor population. Such markers include markers of endothelial lineages, smooth muscle lineages and cardiomyocyte lineages, are well known by persons of ordinary skill in the art, and include, but are not limited to, PECAM1, flk1, CD31, VE-cadherin, CD146, vWF as endothelial cell marker; smooth muscle actin (SMA or SM-actin) or smooth muscle myosin heavy chain (SM-MHC) and response to vasoactive hormone Angotensin II as smooth muscle markers; acetylcholinesterase (Ach-esterase) troponin (TnT), TnT1, β-actinin, atrial natruic factor (ANF) as cardiomyocyte markers. In further embodiments, other useful markers for positive selection of cardiomyocytes may include, without limitation, one, two or more of NCAM (CD56); HNK-1; L-type calcium channels; cardiac sodium-calcium exchanger; etc. Additional cytoplasmic markers for cardiomyocyte subsets are also of interest, e.g. Mlc2v for ventricular-like working cells; and Anf as a general marker of the working myocardial cells. Markers for pacemaker cells also include HCN2, HCN4, connexin 40, etc.

### IX. Therapeutic Uses of Isl1⁺ Progenitors Produced by the Method of the Present Invention

The isl1⁺ progenitors produced and/or expanded by the methods of the present invention are useful for therapeutic applications for congenital and adult heart failure or for the further development of therapeutics for such applications.

In another aspect, the methods provide use of the isl1⁺ progenitors produced by the methods herein. In one embodiment of the invention, the isl1⁺ progenitors may be used for the production of a composition for use in transplantation into subjects in need of cardiac transplantation, for example, but not limited to subjects with congenital and acquired heart disease and subjects with vascular diseases. In one embodiment, the isl1⁺ progenitors may be genetically modified. In another aspect, the subject may have or be at risk of heart disease and/or vascular disease. In some embodiments, the isl1⁺ progenitors may be autologous and/or allogenic. In some embodiments, the subject is a mammal, and in other embodiments the mammal is a human.

The present invention provides methods of generating and expanding isl1⁺ progenitors that provide advantages over existing methods, because the isl1⁺ progenitors can be obtained from any cell from any tissue. For example, cells derived from tissue, for example heart tissue can be induced to become islet 1+ progenitors by suppressing wnt/β-catenin signalling, which can be subsequently expanded by activation of the wnt/β-catenin signalling. Cells can be, for example, progenitors, stem cells, or cells from fetal, embryonic, postnatal and adult tissue. This is highly advantageous as the methods provided herein permit generation of isl1⁺ progenitors as well as a renewable source of isl1⁺ progenitors from any cell type and any cell origin. In some embodiments, the isl1⁺ progenitors produced by the methods herein, for example isl1⁺ cardiovascular progenitors function as a renewable source of the originating population of cells that can be subsequently induced along specific differentiation pathways to become the desired cell type and/or exhibit or acquire the desired phenotypes and characteristics and properties the cell population is more likely to be. Thus, by differentiating the isl1⁺ progenitors produced by the method described herein, multiple cells for cardiac transplantation can be produced, including, but not limited to cardiac myocytes and cells that make up the coronary arterial tree. A renewable supply of isl1⁺ progenitors that can differentiate into cardiac myocytes types has advantages, as cardiac muscle cells typically have restricted differentiation potential. Thus, using the methods provided herein, permits regeneration of specific heart structures without the risks and limitations of other ES cell based systems, such as risk of teratomas (Lafamme and Murry, 2005, Murry et al, 2005; Rubart and Field, 2006).

In another embodiment, the isl1⁺ progenitors produced by the methods herein, for example isl1⁺ cardiovascular progenitors, can be used as models for studying differentiation pathways of isl1⁺ cardiovascular progenitors and cardiac progenitors into multiple lineages, for example but not limited to cardiac, smooth muscle and endothelial cell lineages. In some embodiments, the isl1⁺ progenitors produced by the methods herein, for example isl1⁺ cardiovascular progenitors, may be genetically engineered to comprise markers operatively linked to promoters that are expressed in one or more of the lineages being studied. In some embodiments, the isl1⁺ progenitors produced by the methods herein, for example isl1⁺ cardiovascular progenitors, can be used as a model for studying the differentiation pathway of cardiovascular stem cells into subpopulations of cardiomyocytes. In some embodiments, isl1⁺ progenitors produced by the methods herein, for example isl1⁺ cardiovascular progenitors may be genetically engineered to comprise markers operatively linked to promoters that drive gene transcription in specific cardiomyocyte subpopulations, for example but not limited to atrial, ventricular, outflow tract and conduction systems. In other embodiments, isl1⁺ progenitors produced by the methods herein, for example isl1⁺ cardiovascular progenitors are derived from tissue, for example but not limited to embryonic heart, fetal heart, postnatal heart and adult heart.

One embodiment relates to a method of treating a circulatory disorder or cardiovascular disorder, comprising administering an effective amount of a composition comprising isl1⁺ progenitors produced by the methods herein. For example, isl1⁺ cardiovascular progenitors are used to treat a subject with a circulatory and/or cardiovascular disorder. In a further embodiment, a method is provided for treating myocardial infarction, the method comprising administering a composition comprising isl1⁺ progenitors produced by the methods herein, to a subject having a myocardial infarction in an effective amount sufficient to produce cardiac muscle cells in the heart of the individual.

The invention further provides for a method of treating an injured tissue in an individual comprising: (a) determining a site of tissue injury in the individual; and (b) administering isl1⁺ progenitors produced by the methods described herein in a composition into and around the site of tissue injury, wherein the composition comprising isl1⁺ progenitors, for example isl1⁺ cardiovascular progenitors are to undergo, or have been differentiated into a cardiac muscle cell or cardiovascular vascular cell, or cardiovascular epithelial cell or coronary arterial tree. In one embodiment, the tissue is cardiac tissue, for example cardiac muscle. In one embodiment, the isl1⁺ progenitors, for example isl1⁺ cardiovascular progenitors are derived from an autologous source. In a further embodiment, the tissue injury is a myocardial infarction, cardiomyopathy or congenital heart disease or a cardiovascular disorder.

In one embodiment of the above methods, the subject is a human and the isl1⁺ progenitors are human cells. In alternative embodiments, isl1⁺ progenitors can be use to treat circulatory disorder or cardiovascular disorder is selected from the group consisting of cardiomyopathy, myocardial infarction, and congenital heart disease. In some embodiments, the circulatory disorder is a myocardial infarction. In some embodiments, the isl1⁺ progenitors produced by the methods described herein are differentiated into cardiac muscle cells, and can be used for treating myocardial infarction by reducing the size of the myocardial infarct. It is also contemplated that the differentiation of isl1⁺ progenitors produced by the method of the present invention into a cardiac muscle cell treats myocardial infarction by reducing the size of the scar resulting from the myocardial infarct. The invention contemplates that isl1⁺ progenitors are administered directly to heart tissue of a subject, or are administered systemically.

The present invention is also directed to a method of treating circulatory damage in the heart or peripheral vasculature which occurs as a consequence of genetic defect, physical injury, environmental insult or damage from a stroke, heart attack or cardiovascular disease (most often due to ischemia) in a subject, the method comprising administering (including transplanting), an effective number or amount of isl1⁺ progenitors produced by the methods as described herein to a subject. Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

The term “effective amount” as used herein refers to the amount of therapeutic agent of pharmaceutical composition to reduce at least one or more symptom(s) of the disease or disorder, and relates to a sufficient amount of pharmacological composition to provide the desired effect. The phrase “therapeutically effective amount” as used herein, e.g., of Isl1+ progenitors disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylatically significant reduction in a symptom or clinical marker associated with a cardiac dysfunction or disorder when administered to a typical subject who has a cardiovascular condition, disease or disorder.

A therapeutically or prophylatically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

With reference to the treatment of a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be monitored by clinically accepted criteria; such as, for example, a reduction of area occupied by scar tissue or revascularization of scar tissue, and in the frequency and severity of angina, or an improvement in developed pressure, systolic pressure, end diastolic pressure, patient morbidly and quality of life. Efficacy of treatment can also be evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals. By “earlier” is meant that a decrease, for example in the size of the tumor occurs at least 5% earlier, but preferably more, e.g., one day earlier, two days earlier, 3 days earlier, or more.

As used herein, the term “treating” when used in reference to a cancer treatment is used to refer to the reduction of a symptom and/or a biochemical marker of cancer, for example a reduction in at least one biochemical marker of cancer by at least about 10% would be considered an effective treatment. Examples of such biochemical markers of cardiovascular disease include reduction of area occupied by scar tissue or revascularization of scar tissue, and in the frequency and severity of angina, or an improvement in developed pressure, systolic pressure, end diastolic pressure, patient morbidly and quality of life. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

In some embodiments, the effects of cell delivery therapy would be demonstrated by, but not limited to, one or more of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of cellular therapy can be evident over the course of days to weeks after the procedure. However, beneficial effects may be observed as early as several hours after the procedure, and may persist for several years.

The differentiated cells may be used for tissue reconstitution or regeneration in a human patient or other subject in need of such treatment. The cells are administered in a manner that permits them to graft or migrate to the intended tissue site and reconstitute or regenerate the functionally deficient area. Special devices and/or cell scaffolds or matrices are available that are adapted for administering cells capable of reconstituting cardiac function directly to the chambers of the heart, the pericardium, or the interior of the cardiac muscle at the desired location. The cells may be administered to a recipient heart by intracoronary injection, e.g. into the coronary circulation. The cells may also be administered by intramuscular injection into the wall of the heart.

The compositions comprising isl1⁺ progenitors produced by the methods described herein have a variety of uses in clinical therapy, research, development, and commercial purposes. For therapeutic purposes, for example, isl1⁺ progenitors and their progeny may be administered to enhance tissue maintenance or repair of cardiac muscle for any perceived need, such as an inborn error in metabolic function, the effect of a disease condition, or the result of significant trauma. The isl1⁺ progenitors that are administered to the subject not only help restore function to damaged or otherwise unhealthy tissues, but also facilitate remodeling of the damaged tissues, and/or protect or prevent development of a pathological condition.

To determine the suitability of a composition comprising isl1⁺ progenitors for therapeutic administration, the isl1⁺ progenitors can first be tested in a suitable animal model. At one level, cells are assessed for their ability to survive and maintain their desired phenotype in vivo. Cell compositions can be administered to immunodeficient and/or immunocompromised animals (such as nude mice, or animals rendered immunodeficient chemically or by irradiation). Tissues are harvested after a period of regrowth, and assessed as to whether the administered cells or progeny thereof are still present, and/or of the desired phenotype.

This can be performed by administering isl1⁺ progenitors that express a detectable label (such as green fluorescent protein, or beta-galactosidase, that have been prelabeled (for example, with BrdU or [3H] thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

Where the isl1⁺ progenitors produced and/or expanded by the methods of the present invention are differentiated towards a cardiomyocyte lineage, suitability can also be determined in an animal model by assessing the degree of cardiac recuperation that ensues from treatment with the differentiating cells of the invention. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N2 on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

The isl1⁺ progenitors produced and/or expanded by the methods of the present invention may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or feeder cells associated with progenitor cell proliferation and differentiation.

The isl1⁺ progenitors produced and/or expanded by the methods of the present invention can be supplied in the form of a composition comprising an isotonic excipient prepared under sufficiently sterile conditions for human administration. For general principles in medicinal formulation, the reader is referred to Stem Cells: Handbook of Experimental Pharmacology, Anna M. Wobus (Editor), Kenneth R. Boheler (Editor) Springer Press, 2005 and Embryonic Stem Cell Protocols: Differentiation Models by Kursad Turksen (Editor) Humana Press; 2006; Embryonic Stem Cell Protocols: Isolation And Characterization by Kursad Turksen Humana Press; 2nd Ed, 2006; Stem Cells Handbook, S. Sell, ed., Humana Press, 2003. Embryonic Stem Cells: Methods and Protocols K. Turksen, ed., Humana Press, 2002, and Human Embryonic Stem Cells by A. Chiu and M. Rao, ed., Humana Press, 2003;

Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells. In another embodiment, the composition may comprise resorbable or biodegradable matrix scaffolds.

In some embodiments, the isl1⁺ progenitors produced and/or expanded by the methods described herein may be genetically altered in order to introduce genes useful in the differentiated cell, e.g. repair of a genetic defect in an individual, selectable marker, etc., or genes useful in selection against undifferentiated ES cells. Isl1⁺ progenitors produced by the methods described herein may also be genetically modified to enhance survival, control proliferation, and the like. Isl1⁺ progenitors may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Isl1⁺ progenitors may be genetically altered using vector containing supernatants over a 8-16 h period, and then exchanged into growth medium for 1-2 days. Genetically altered isl1⁺ progenitors are selected using a methods commonly known in the art, for example, using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e. prevent rejection).

In an alternative embodiment, the isl1⁺ progenitors produced and/or expanded by the methods of the present invention can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, cardiotropic factors such as atrial natriuretic factor, cripto, and cardiac transcription regulation factors, such as GATA-4, Nkx2.5, and Mef2-C.

Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bcl-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

In one aspect of the present invention, the isl1⁺ progenitors produced by the methods of the present invention are suitable for administering systemically or to a target anatomical site. The isl1⁺ progenitors can be grafted into or nearby a subject's heart, for example, or may be administered systemically, such as, but not limited to, intra-arterial or intravenous administration. In alternative embodiments, the isl1⁺ progenitors can be administered in various ways as would be appropriate to implant a composition comprising isl1⁺ progenitors, including but not limited to parenteral, including intravenous and intraarterial administration, intrathecal administration, intraventricular administration, intraparenchymal, intracranial, intracisternal, intrastriatal, and intranigral administration. Optionally, the isl1⁺ progenitors are administered in conjunction with an immunosuppressive agent.

The isl1⁺ progenitors produced by the methods of the present invention can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. The delivery of the isl1⁺ progenitors produced and/or expanded by the methods of the present invention may take place but is not limited to the following locations: clinic, clinical office, emergency department, hospital ward, intensive care unit, operating room, catheterization suites, and radiologic suites.

In other embodiments, at least a portion of the isl1⁺ progenitors produced and/or expanded by the methods of the present invention are stored for future expansion and/or subsequent implantation. The isl1⁺ progenitors may be divided into more than one aliquot or unit such that part of the population of isl1⁺ progenitors are retained for later application, while part can be applied immediately to the subject. Moderate to long-term storage of all or part of the isl1⁺ progenitors produced and/or expanded by the methods of the present invention is encompassed in the methods described herein, with isl1⁺ progenitors stored, for example, in a cell bank as disclosed in U.S. Patent Application Serial No. 20030054331 and Patent Application No. WO03/024215, which are incorporated herein by reference in their entirety. At the end of processing, the isl1⁺ progenitors may be loaded into a delivery device, such as a syringe, for placement into the recipient by any means known to one of ordinary skill in the art.

### X. Pharmaceutical Composition

The compositions may comprise isl1⁺ progenitors produced and/or expanded by the methods of the present invention, and can optionally comprise at least one differentiating agent. Differentiation agents for use in the methods described are well known to those of ordinary skill in the art. The compositions may further comprise a pharmaceutically acceptable carrier.

The isl1⁺ progenitors produced and/or expanded by the methods of the present invention may be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. The cell population may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, the isl1⁺ progenitors produced and/or expanded by the methods described herein can be combined with a gene encoding pro-angiogenic and/or cardiomyogenic growth factor(s) which would allow cells to act as their own source of growth factor during cardiac repair or regeneration. Genes encoding anti-apoptotic factors or agents could also be applied. Addition of the gene (or combination of genes) can be by any technology known in the art including but not limited to adenoviral transduction, “gene guns,” liposome-mediated transduction, and retrovirus or lentivirus-mediated transduction, plasmid' adeno-associated virus. Isl1⁺ progenitors produced by the methods described herein can be implanted along with a carrier material bearing gene delivery vehicle capable of releasing and/or presenting genes to the isl1⁺ progenitors over time such that transduction can continue or be initiated.

In one embodiment, the methods described herein reduce and/or eliminate the need for allogenic cell transplantation, as the isl1⁺ progenitors can be induced to form from any cell and/or tissue type, including for example fetal and adult tissue obtained from a subject and expanded without losing multi-lineage differentiation potential. In some embodiments, a cell taken from a subject can be induced along islet 1 lineages and expanded using the methods described herein and used, for example, in the treatment of a cardiac disorder or cardiovascular disorder by being transplanted back into the subject from which the cell was originally derived. In some embodiments, when the isl1⁺ progenitors produced and expanded by the methods described herein are administered to a subject other than the subject from whom the original cell and/or tissue used to generate the isl1⁺ progenitors were obtained, one or more immunosuppressive agents may be administered to the subject receiving the isl1⁺ progenitors and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 20020182211. A preferred immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiovascular stem cells of the invention.

In certain embodiments, the isl1⁺ progenitors produced and/or expanded by the present invention are administered to a patient with one or more cellular differentiation agents, such as cytokines and growth factors. Examples of various cell differentiation agents are disclosed in Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; and Zuk et al., 2001.

Compositions comprising effective amounts of isl1⁺ progenitors produced and/or expanded by the present invention are also contemplated by the present invention. These compositions comprise an effective number of cells, optionally, in combination with a pharmaceutically acceptable carrier, additive or excipient. In certain aspects, cells are administered to the subject in need of a transplant in sterile saline. In other aspects, the isl1⁺ progenitors produced and expanded by the methods described herein are administered in Hanks Balanced Salt Solution (HBSS) or Isolyte S, pH 7.4. Other approaches may also be used, including the use of serum free cellular media. In one embodiment, the isl1⁺ progenitors produced and expanded by the methods described herein are administered in plasma or fetal bovine serum, and DMSO. Systemic administration of the isl1⁺ progenitors produced and/or expanded by the methods herein to the subject may be preferred in certain indications, whereas direct administration at the site of or in proximity to the diseased and/or damaged tissue may be preferred in other indications.

The composition may optionally be packaged in a suitable container with written instructions for a desired purpose, such as the reconstitution of cardiomyocyte cell function to improve some abnormality of the cardiac muscle.

In one embodiment, the isl1⁺ progenitors produced and/or expanded by the methods herein are administered with a differentiation agent. In one embodiment, the isl1⁺ progenitors are combined with the differentiation agent to administration into the subject. In another embodiment, the isl1⁺ progenitors are administered separately to the subject from the differentiation agent. Optionally, if the isl1⁺ progenitors are administered separately from the differentiation agent, there is a temporal separation in the administration of the isl1⁺ progenitors and the differentiation agent. The temporal separation may range from about less than a minute in time, to about hours or days in time. The determination of the optimal timing and order of administration is readily and routinely determined by one of ordinary skill in the art.

### XI. Other Uses of Isl1⁺ Progenitors Generated and Expanded by the Methods of the Present Invention

In some embodiments, a hierarchy isl1⁺ progenitors produced and/or expanded by the methods described herein can be easily manipulated in experimental systems that offer the advantages of targeted lineage differentiation as well as clonal homogeneity and the ability to manipulate external environments. Furthermore, due to ethical unacceptability of experimentally altering a human germ line, the ES cell transgenic route is not available for experiments that involve the manipulation of human genes. Gene targeting in isl1⁺ progenitors produced and/or expanded by the methods described herein, for example, isl1⁺ cardiovascular progenitors allows important applications in areas where rodent model systems do not adequately recapitulate human biology or disease processes.

In one embodiment, the isl1⁺ progenitors produced and/or expanded by the methods herein can be used to assess the affect of other agents and/or chemicals on their renewal and differentiation. For example, using the isl1⁺ progenitors of the present invention one can evaluate candidate drugs, for example to evaluate toxicity and/or efficacy of candidate drugs, therapies and agents. For example, the isl1⁺ progenitors produced and/or expanded by the methods can be used in assays for cardiotoxic testing on isl1⁺ cardiovascular progenitors etc. Candidate agents include, organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more functional groups. Candidate agents are also found among biomolecules, including peptides, polynucleotides, saccharides, fatty acids, steroids, purines, pyrimidines, derivatives, structural analogs or combinations thereof.

In one embodiment, a plurality of hierarchical isl1⁺ progenitors produced and/or expanded by the methods of the present invention can be used in an assay for the study and understanding of signaling pathways of the islet 1 lineage pathway, for example signals triggering uncommitted progenitors to enter the islet 1 lineage, the renewal of the hierarchical isl1⁺ progenitors, in particular isl1⁺ cardiovascular progenitors, and their downstream differentiation. The isl1⁺ progenitors produced and/or expanded by the methods described herein are useful in aiding the development of therapeutic applications for congenital and adult heart failure. The isl1⁺ progenitors produced by the methods herein can be used to study specific cardiac lineages in particular cardiac structures without the need and complexity of time consuming animal models. In another embodiment, the cells can be genetically modified to carry specific disease and/or pathological traits and phenotypes of cardiac disease and adult heart failure.

In another embodiment, the assay comprises a plurality of cardiovascular stem cells, or their differentiated progeny. In one embodiment, the assay comprises cells derived from the cardiovascular stem cells described herein. In one embodiment, the assay can be used to study differentiation pathways of cardiovascular stem cells, for example but not limited to differentiation along the lineages of cardiomyocyte differentiation, smooth muscle differentiation, endothelial differentiation, and subpopulations of these lineages. In one embodiment, the study of subpopulations can be, for example, study of subpopulations of cardiomyocytes, for example atrial cardiomyocytes, ventricular cardiomyocytes, outflow tract cardiomyocytes, conduction system cardiomyocytes, and coronary arterial tree differentiation.

In another embodiment, an assay can be used to study isl1⁺ progenitors produced and/or expanded by the methods herein, for example the isl1⁺ cardiovascular progenitors which comprise a pathological characteristic, for example, a disease and/or genetic characteristic associated with a disease or disorder. In some embodiments, the disease of disorder is a cardiovascular disorder or disease. In some embodiments, the cardiovascular stem cell has been genetically engineered to comprise the characteristic associated with a disease or disorder. Such methods to genetically engineer the cardiovascular stem cell are well known by those in the art, and include introducing nucleic acids into the cell by means of transfection, for example but not limited to use of viral vectors or by other means known in the art.

In another embodiment, the isl1⁺ progenitors produced and/or expanded by the methods described herein can be used to prepare a cDNA library relatively uncontaminated with cDNA that is preferentially expressed in cells from other lineages. For example, human isl1⁺ progenitors, for example isl1⁺ cardiovascular progenitors are collected and then mRNA is prepared from the pellet by standard techniques (Sambrook et al., supra). After reverse transcribing into cDNA, the preparation can be subtracted with cDNA from other undifferentiated ES cells, other progenitor cells, or end-stage cells from the cardiomyocyte or any other developmental pathway, for example, in a subtraction cDNA library procedure.

The present invention is further illustrated by the following examples which in no way should be construed as being further limiting. The contents of all cited references, including literature references, issued patents, published patent applications, and co-pending patent applications, cited throughout this application are hereby expressly incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

Progenitors and High-Throughput Chemical Screening. Postnatal cardiac progenitors were isolated as previously described (Laugwitz et al., 2005). Briefly, 30-40 hearts from 1-5 day-old isl1-mER-Cre-mER/R26R pups were used to prepare CMC, containing isl1+ progenitors marked by β-galactosidase. 4-OH-TM (Sigma), active form of tamoxifen, was added to the culture 1 day after plating at 1 μm. These were expanded for 7 days, trypsinized, and seeded at a density of 3,000 cells per well in 384-well plates. Cells were then treated for 4 days with a DMSO control or small molecule from the chemical library described previously by Ding et al., 2002. β-galactosidase in the cell lysate was quantified by luciferase activity using the Beto-Glo assay kit (Promega).

Production of New Mouse Lines. A 3.97 kb Mef2c anterior heart field (AHF) specific enhancer/promoter element (Dodou et al. 2004) was kindly provided by Dr. Brian Black (UCSF). This element was cloned into a promoterless eGFP-1 vector (Clontech, CA). The AHF enhancer/promoter together with the eGFP expression sequence and polyA tail were introduced into the pronucleus from C57B1/6C3 F1 mice. Two founder males were expanded. These males were mated with wild type C57B1/6 females and the cardiac specific GFP expression phenotype was confirmed by examination of AHF-GFP+ embryos. Floxed β-catenin mice were obtained purchased from Jackson lab. Isl1-MerCreMer(MCM) mice were created in Dr. Evans' lab. Homozygous floxed β-catenin mice were crossed with Protamine-Cre mice (O'Gorman, S, et al 1997) to generate β-catenin +/−mice, which were then crossed with isl1-MCM mice to produce doubly heterozygous isl1-MCM/+; β-catenin +/−mice. These mice were then crossed to β-catenin floxed/floxed homozygous mice to obtain isl1-MCM/+; β-cat−/f mutants for analysis. Tamoxifen (1 mg/female, Sigma) was injected at pregnancy females E9.5 and embryos were harvest at E11.5.

Generation of AHF-GFP ES Cell Lines. Timed matings were performed between AHF-GFP transgenic males and C57B1/6 females. On day 3.5 PC, the females were sacked and the blastocysts flushed from the uterine horns using M2 medium (Sigma-Aldrich, MO). After washing with M2 media, the zona pellucida was removed with acidic Tyrode's Solution (Sigma-Aldrich, MO) and the blastocysts were further washed three times in M2 media. The blastocysts were then adapted onto mouse embryonic feeder cells (MEF) with derivation media (DMEM with 15% KOSR, pen/strep, pyruvate, nonessential amino acids, and leukemia inhibitory factor [LIF] [Chemicon, CA]).

In Vitro Differentiation of ES Cell-Derived AHF-GFP Cells. ES cells were maintained in culture in maintenance media (DMEM, 20% FCS, pen/strep, NEAA, pyruvate, L-Glutamine and LIF) and adapted on gelatin-coated plates in the presence of LIF for 2 days prior to differentiation. At day 0 of differentiation, cells were dissociated with 0.25% trypsin and 0.05% EDTA and differentiation was induced by forming embryoid bodies (EB's) in hanging drops of 600 cells in 15 μL of media without LIF. On day 6, the EB's were trypsinized into single cell suspension and sorted on the basis of GFP expression using flow cytometry. In order to test for the effect of Wnt/β-catenin signalling on differentiation, sorted GFP+ cells were plated on fibronectin-coated slides in the presence of conditioned media from L cells stably expressing Wnt3a or from control cells. After 3 days in culture, the cells were fixed in 3.7% formaldehyde stained for troponin T expression.

RNA Isolation and Quantitative PCR. Cells were washed in PBS, pelleted and total RNA was purified from each sample using the Micro RNA Isolation Kit from Stratagene (La Jolla, Calif.). cDNA was made using the iScript cDNA synthesis kit (BioRad, CA), and quantitative PCR was performed using the iQ SYBR Green Supermix (BioRad, CA) on an Eppendorf Mastercycler for 40 cycles. Primer sequences are available upon request.

Isolation of Murine Postnatal Cardiac Progenitors and High-throughput Chemical Screening. Postnatal cardiac progenitors were isolated as previously described (Laugwitz et al., 2005). Briefly, 30-40 hearts from 1-5 day-old is1-mER-Cre-mER/R26R pups were used to prepare CMC, containing isl1⁺ progenitors marked by β-galactosidase. 4-OH-TM (Sigma), active form of tamoxifen, was added to the culture 1 day after plating at 1 μm. These were expanded for 7 days, then trypsinized, and seeded at a density of 3,000 cells per well in a 384-well plate. Cells were then treated for 4 days with a DMSO control or small molecule from the chemical library described previously by Ding et al., 2002. β-galactosidase in the cell lysate were quantified by luciferase activities using the Beto-Glo assay kit (Promega).

ES Cell Culture and Differentiation. The anterior heart field MEF2-GFP ES cells were generated as following. The 3.97 kb Mef2c anterior heart field enhancer element was kindly provided by Brian Black. This fragment was cloned into the pEGFP-1 promoter-less vector (Clontech, CA). The construct was linearized with Xho1 and electroporated into CGR8 ES cells (Kindly provided by Richard Lee, Brigham and Women's Hospital, Boston). Clones were selected with 200 mg/ml of G418 and were assayed for genomic integration of the anterior heart field enhancer by PCR and selected for their GFP expression using inverted microscopy. AHF-GFP positive CGR8 ES cells were maintained in culture in GMEM supplemented with 15% knockout serum (Invitrogen, CA), pyruvate, pen-strep, non-essential amino acids, β-mercaptoethanol, and leukaemia inhibitory factor (Chemicon, CA). At day 0 of differentiation, cells were dissociated with 0.25% trypsin and 0.05% EDTA. Differentiation was induced by forming embryoid bodies (EB's) in hanging drops of 600 cells in 15 μL of media without LIF. On day 6, the EB's were trypsinized into single cell suspension and sorted on the basis of GFP expression using flow cytometry. GFP positive cells were plated on a neonatal cardiac mesenchymal feeder layer for 7 days with one of four conditions: DMSO, BIO, and conditioned media from either control cells (L-Cells) or cells overexpressing wnt3A. After 7 days of expansion on the cardiac mesenchymal feeder layer, the cells were trypsinized and FACS sorted. Differentiation of the GFP positive cells was triggered as follow: into myocytes, on fibronectin by using DMEM/M199 (4:1 ratio) medium containing 10% horse serum and 5% FBS; into SM cells, on fibronectin by using DMEM/F12 containing B27 supplement, 2% FBS, and 10 ng/ml EGF. The culture and differentiation conditions for Isl1-nLacZ knock-in ES cells were described previously (Moretti et al., 2006).

Isolation, Amplication and Differentiation of Embryonic Cardiovascular Progenitor Cells. For isolation of embryonic cardiovascular progenitors, we crossed isl1-IRES-Cre mice (generously provided by Thomas M. Jessel) into the Cre reporter strain Z/RED (Vintersten et al., 2004). Approximately 80 ED 8.5 embryos were dissected and dissociated into single cells by the treatment with a mix of 1 ml collagenase A&B (Roche) at 10 mg/ml for 1 hour at 37° C. followed by a subsequent treatment with trypsin 0.25% for 5-10 min. The dissociated cells were filtered through a 40 inn cell strainer (Falcon) and plated as single cells on the mitomycin-treated feeder layers, stably transfected with Wnt3a, at a density of 10,000 cells/cm² in DMEM/F12 complete media for 7 days. Embryonic cardiovascular progenitors were sorted based on the DsRed expression. Smooth muscle spontaneous differentiation was performed as previously described (Moretti et al., 2006). Briefly, FACS-sorted DsRed⁺ cells were plated at a density of 5,000 cells/cm² on fibronectin-coated chamber slide or 384-well plate and cultured in DMEM/F12 containing B27 supplement, 2% FBS, and 10 ng/ml EGF (complete progenitor medium) for 1-7 days followed by immunostaining for smooth muscle markers.

Isolation and Cell Culture Conditions of Human Postnatal Cardiac Progenitors. Biopsies are cut in small pieces and washed in solution A (10 mM Hepes, 35 mM NaCl, 10 mM glucose, 134 mM sucrose, 16 mM Na2HPO4, 25 mM NaHCO3, 7.75 mM KCl, 1.18 mM KH2PO4, pH 7.4), supplemented with 30 mM 2,3 butanedione 2-monoxime and 0.5 mM EGTA. First digestion step is performed in solution A supplemented with 0.5% BSA, 200 UI/ml of collagenase type II (Worthington) and 6 UI/ml protease type XXIV (Sigma) for 20 min at 37° C. to remove red blood cells and cell debris. Four digestion steps are performed in solution A supplemented with 400 UI/ml collagenase type V for 20 min at 37° C. and centrifuged at 30×g for 1 min. The supernatant is neutralized from the collagenase by adding ⅕ of NCS (newborn calf serum) and are centrifuged at 1300 rpm for 3-5 min. Pellet is resuspended in DMEM supplemented with 10% NCS, 5% FBS and Pen-Strep, and cells are seeded on chamber slide. BIO was added to the culture at different doses in complete progenitor medium and cultured for 4 days prior to immunostaining for Isl1.

Production of Reagents for Wnt Pathway. Wnt3a or control-conditioned medium was produced as following. A Wnt3a-secreting cell line (ATCC) was allowed to grow to conflency and subcultured at 1:20 ratio prior to replenish with fresh medium. Three batches of conditioned medium were harvested every 48 hrs. Dkk1-conditioned medium was produced by transiently transfecting a Dkk1-expressing cDNA (generously provided by Dr. Randall T. Moon) into the HEK293T cell line with FuGENE 6 (Roche). The supernatant was harvested 72 hrs after transfection. A Wnt-reporter cell line, superTOPFLASH, was a generous gift from Dr. Randall T. Moon.

Small hairpin RNAs (shRNAs) oligonucleotide sequences were designed based on the following target sequences: siWLS-A (CACAAATCCTTTCTACAGTAT) (SEQ ID NO:1) and siWLS-B (GGGTTACCGTGATGATATG, (SEQ ID NO:2) and subcloned into the retro-viral vector, RNAi-Ready pSIREN-RetroQ-DsRed-Express (Clontech) according to vendor's instructions. Negative control shRNA annealed oligonucleotide was provided by the vendor and cloned to the same vector. Retro-viral siRNAi particles were produced by transiently transfect these vectors into the packaging cell line EcoPack 2-293 cells with GENE 6 (Roche). Viral supernatant was harvested 72 hrs after transfection.

Immunohistochemical Analyses and lacZ Staining. Cells in culture or paraffin embedded sections were fixed with 4% paraformaldehyde and subjected to immunostaining. For mouse embryo cryosections, they were saturated with 20% sucrose followed by section and 2% PFA fix for 10 min at room temperature. The following primary antibodies were used in this study: isl1 (mouse monoclonal antibody, clone 39.4D5, clone 2D6 (Developmental Studies Hybridoma Bank, 1:100), cardiac troponin T (mouse monoclonal antibody, NeoMarkers, 1:200), smooth muscle myosin heavy chain (rabbit polyclonal, Biomedical Technologies Inc., 1:100), smooth muscle actin (mouse monoclonal, clone 1A4, Dako, 1:100; rabbit polyclonal, Abcam), phosphohistone H3 (rabbit polyclonal, Upstate). Alexa Fluor 488- or Alexa Fluor 594-conjugated secondary antibodies specific to the appropriate species were used (Molecular Probes, 1:350). 3D reconstruction was done using Winsuf from Surfdriver Software. For immunoperoxidase staining, the VECTASTAIN ABC® system (VECTOR Laboratories) was used, accordingly to the vendors' instructions. 5 μm frozen sections and cultured cells were fixed with 0.2% and 0.05% glutaraldehyde respectively for 10 min at 4° C. followed by 3 times wash with PBS. LacZ stainings were then performed on these samples by incubating with X-Gal solution containing 40 mM HEPES, pH 7.4, 5 mM K3(Fe(CN)₆), 5 mM K4(Fe(CN)₆), 2 mM MgCl₂, 15 mM NaCl, and 1 mg/ml X-Gal. For LacZ staining on EB-derived clones expanded on CMC, 0.02% NP-40 was added to the X-Gal solution for better permeabilization. When combining LacZ staining with immunoperoxidase analyses, samples were processed first for LacZ staining at room temperature over night, followed by a re-fixation with 4% paraformaldehyde prior to the immunoperoxidase stainings for specific epitopes or by re-fixation with 2% gluteraldehyde to perform EM studies.

Statistic Analysis. Data were analyzed with two-tailed Student's t test and the results reported are statistically significant with p value <0.05. Standard error of the mean (SEM) is given for each mean value. For immunofluorescent analyses for postnatal isl1⁺ cardiovascular progenitors, the cells within the whole well in an 8-well chamber slide were examined for Isl1 expression to obtain better statistic comparison.

### Example 1

Neonatal Isl1⁺ Cardiovascular Progenitors Are Preferentially Localized in an In vivo Microenvironment of Cardiac Mesenchymal Cells in the Non-myocyte Compartment. Isl1⁺ cardiac progenitors have recently been identified from rat, mouse and human myocardium with the potential to differentiate into mature atrial and ventricular myocytes (Laugwitz et al., 2005). However, their number decreases progressively after the formation of the heart from embryonic day 12.5 (ED12.5) to adulthood (Laugwitz et al., 2005). As the micro-environmental niche plays a paramount role in stem cell/progenitor maintenance (Scadden, 2006), the inventors analyzed the in vivo microenvironment of isl1⁺ cardiovascular progenitors and the molecular cues that control their formation, renewal, and differentiation that emanates from this microenvironment.

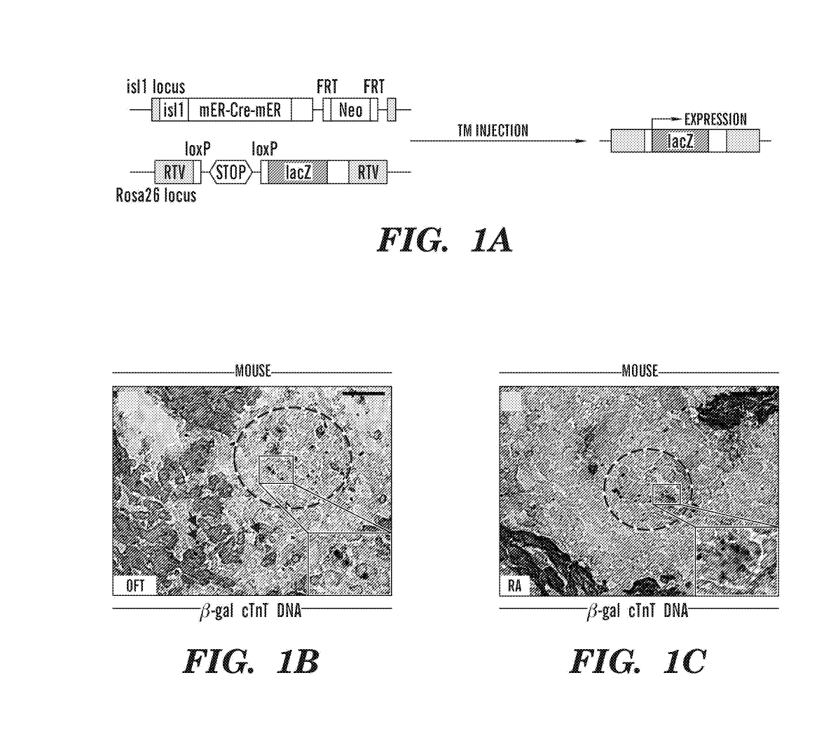

To genetically mark isl1⁺ progenitors in the postnatal heart, the inventors crossed isl1-mER-Cre-mER mice, which express a tamoxifen-inducible Cre recombinase protein fused to two mutated estrogen-receptors under the control of the endogenous isl1 promoter, into the conditional Cre reporter strain R26R (Laugwitz et al., 2005; Soriano, 1999). In the double heterozygous progeny (isl1-mER-Cre-mER/R26R), administration of tamoxifen induces a rapid nuclear translocation of the mER-Cre-mER protein, which allows Cre-mediated recombination leading to the removal of a stop sequence and a ubiquitous expression of the lacZ gene under the control of the endogenous rosa26 promoter (Laugwitz et al., 2005) and FIG. 1A). Thus, cardiac progenitors expressing isl1 at the time of tamoxifen exposure can be faithfully marked by β-galactosidase (β-gal) expression. The inventor demonstrated that isl1⁺ progenitors, particularly isl1⁺ cell clusters, are preferentially localized in a microenvironment composed of cells of a non-myocytic nature, acting as an insulator, and thereby allowing expansion of isl1⁺ cell clusters. In addition, previous studies have shown that the cardiac mesenchymal cells within the non-myocyte compartment serve as an effective microenvironment to allow marked renewal of the post-natal islet progenitors (Laugwitz et al., 2005). β-gal⁺ cells marked by 5-bromo-4-chloro-3-indolyl-β-D-galactoside (X-gal) staining were observed in the vicinity of the outflow tract area with β-gal⁺ cell clusters surrounded by non-myocytic cells shown by their negative staining for cardiac troponin T (FIG. 1B). In addition, such β-gal⁺ clusters can also be seen adjacent to the heart in the dorsal-anterior direction (FIG. 1C).

The inventors next examined the microenvironments of the human neonatal isl1⁺ progenitors in right atrial tissue by double immunofluorescent staining of Isl1 (green) and cardiac troponin T (red). A rare population of isl1⁺ progenitors, in some cases, isl1⁺ clusters, was observed primarily in the epicardium, surrounded by non-myocytic nature by cardiac troponin staining (FIG. 1D). Taken together, these observations indicate that isl1⁺ progenitors are preferentially localized in a microenvironment of non-myocytic nature, which permits the expansion of isl1⁺ clusters.

### Example 2

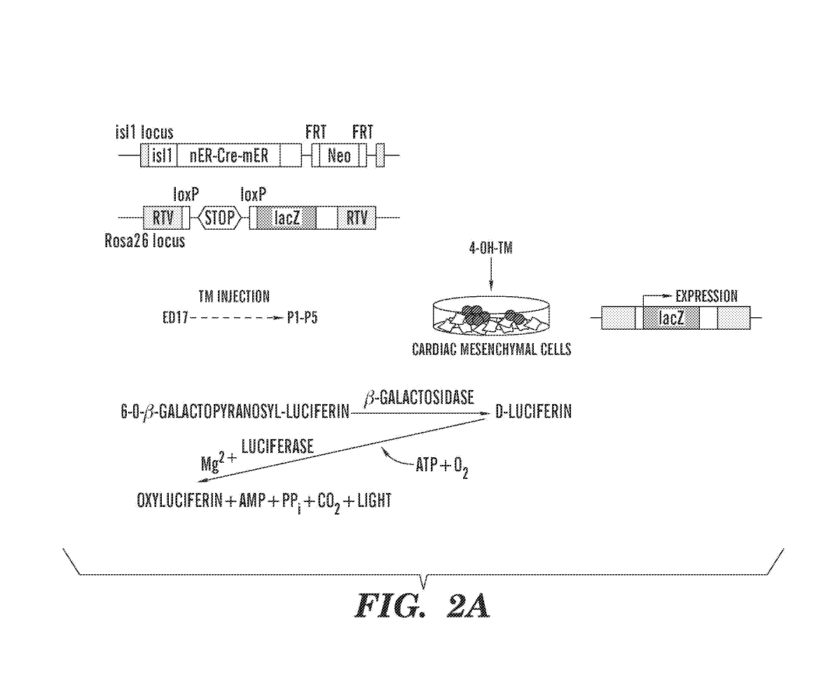

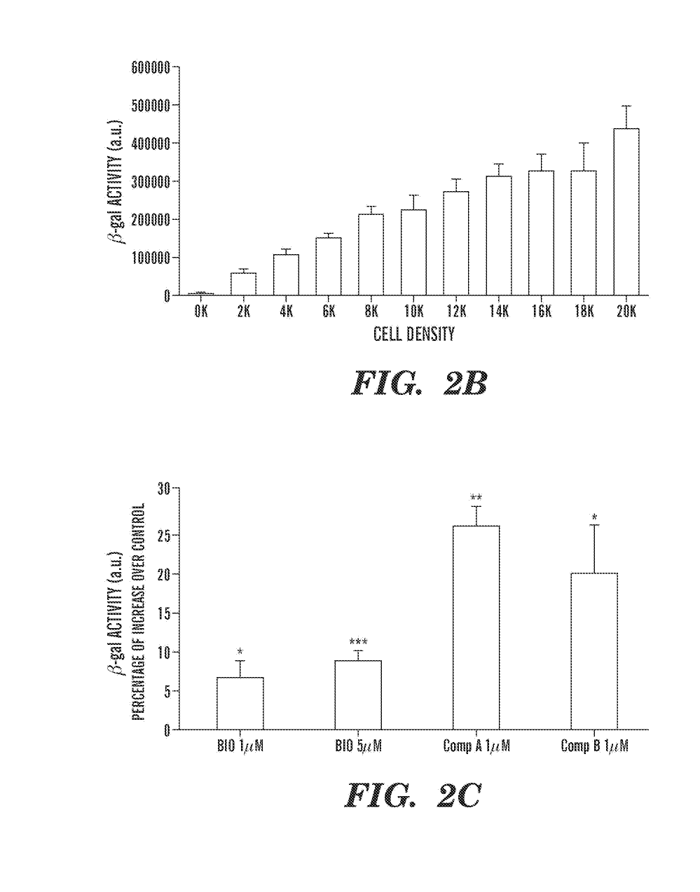

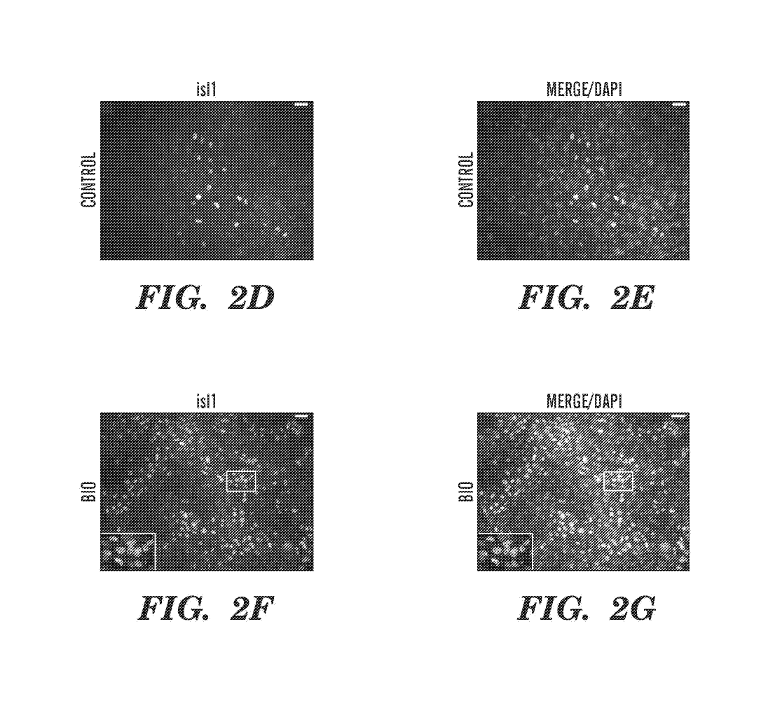

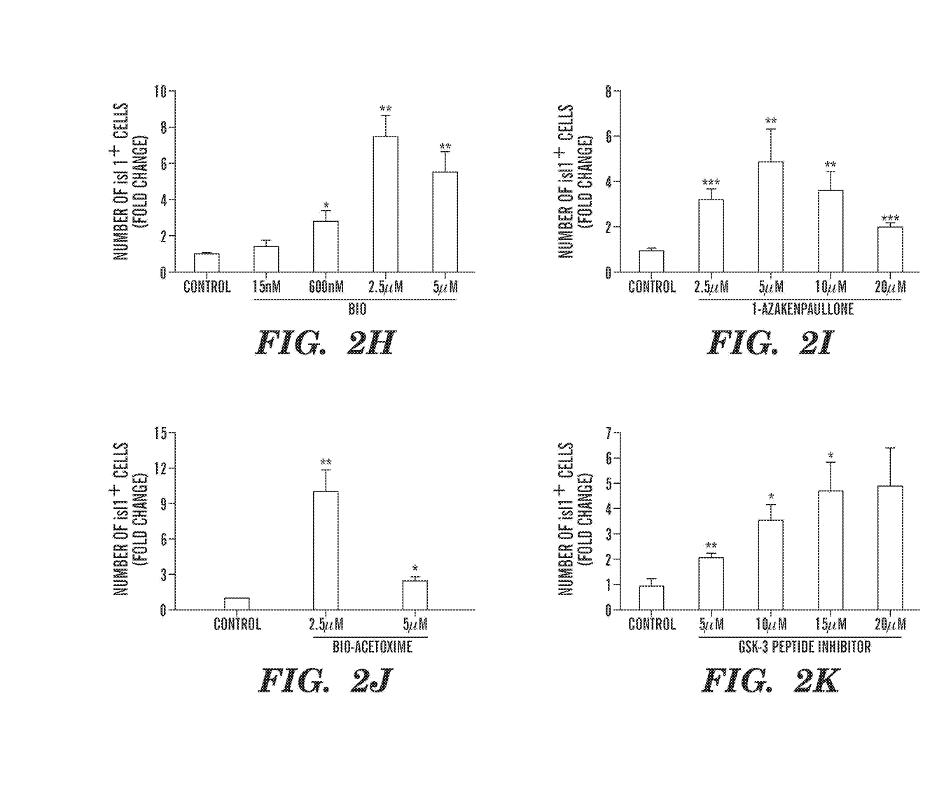

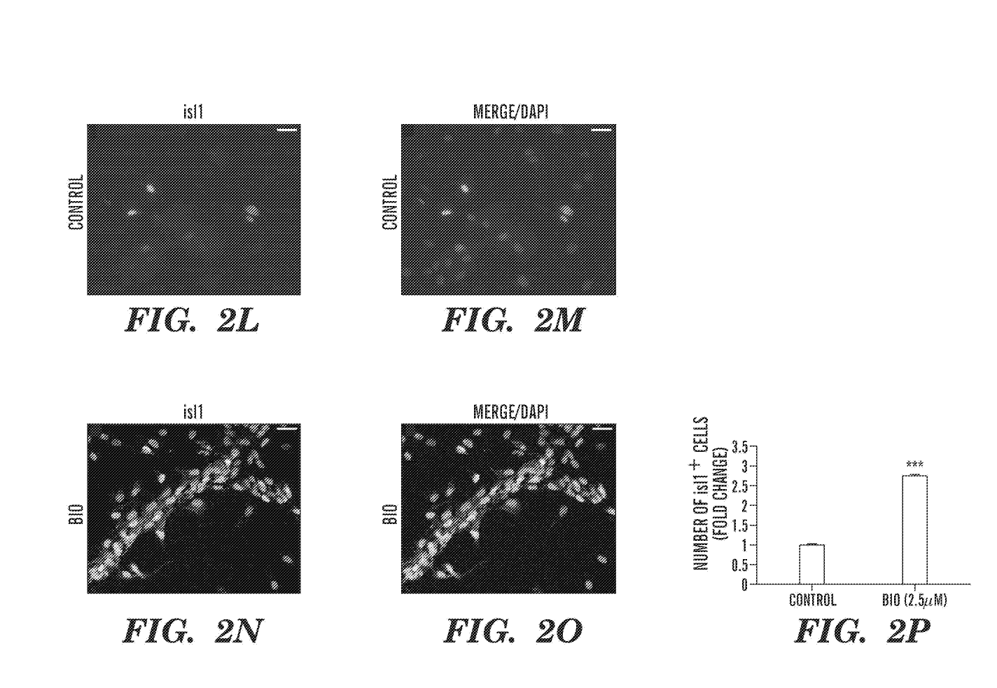

High-Throughput Screening Identifies Chemical Probes that Enhance CMC Cues for Expansion of Isl1+ Cardiac Progenitors. To find cardiac mesenchymal cells (CMC)-derived environmental cues involved in the renewal of isl1+ progenitors, the inventors developed a high-throughput chemical screening system, based on the coculture of CMC with postnatal isl1+ progenitors (FIG. 2A). To genetically mark isl1+ progenitors in the postnatal heart, the inventors crossed isl1-mER-Cre-mER (MCM) mice with the conditional Cre reporter strain R26R (Laugwitz et al., 2005; Soriano, 1999), as shown in FIG. 2A. Recently, several synthetic small molecules from a combinatorial library of heterocyclic compounds were identified that regulate stem cell fate (Ding et al., 2003; Wu et al., 2004). The inventors used this library to screen for small molecules that would expand the rare population of postnatal isl1+ progenitors.

Cardiac mesenchymal cells (CMC) from isl1-MCM/R26R mouse hearts were isolated as previously described (Laugwitz et al., 2005), expanded for 7 days, and treated with a DMSO control or small molecules for an additional 4 days. As seen in FIG. 2B, β-galactosidase (β-gal) activity was directly proportional to the starting amount of CMC.

The screening of over 15,000 independent compounds in four separate experiments identified 25 candidates that were able to significantly upregulate β-gal activity. Although there was only a small increase over the control, the effect of these compounds was highly reproducible and statistically significant (p<0.05, 0.01, or 0.001, FIG. 2C). A more sensitive assay of isl1 immunostaining was performed, and three candidates were noted to substantially increase the number of isl1⁺ progenitors (FIG. 2F and data not shown). Two of these compounds were unknown (compound A and compound B), and the third was 6-bromoindirubin-3′-oxime (BIO), previously shown to be an inhibitor of GSK-3 (Meijer et al., 2003). BIO has been recently shown to promote self-renewal of both human and mouse ES cells through activation of the wnt/β-catenin pathway in combination with other signaling inputs (Sato et al., 2004). The inventors explored the role of BIO on the renewal of isl1⁺ progenitors.

As shown in FIGS. 2D-2H, BIO increased the number of isl1⁺ progenitors in a dose-dependent manner, and a maximal effect was seen at 2.5 mM with an 7-fold increase versus control. BIO also promoted isl1⁺ progenitors to form large clusters, demonstrating BIO promotes proliferation (FIG. 2F). Such clusters were rarely seen in control treated samples, which showed scattered isl1⁺ progenitors (FIG. 2D). Correspondingly, the effect of BIO at 2.5 μM on cluster formation of isl1⁺ progenitors was more appreciable than that observed as single cells (11.6 fold increase versus control, FIG. 2H). The dose dependence of BIO on expanding isl1⁺ progenitors demonstrates that the effect is specific to the action of BIO.

Immunostaining of cleaved caspase-3 showed no appreciable apoptosis in both BIO- and DMSO-treated CMC (data not shown), demonstrating that the expansion of isl1⁺ progenitors by BIO does not occur through repressing apoptosis. To further validate the specificity of BIO function, the inventors tested two other ATP-competitive GSK-3-specific inhibitors: an acetoxime analog of BIO and 1-Azakenpaullone (Kunick et al., 2004; Meijer et al., 2003). Both of these compounds substantially increased isl1⁺ progenitor cell number versus control (FIGS. 2I and 2K). In another experiment, the inventors used a cell-permeable and substrate-competitive GSK-3 peptide inhibitor, which has a negligible inhibitory effect on other protein kinases (Plotkin et al., 2003), was able to significantly expand isl1+ progenitor cells (FIG. 2K). Taken together, the inventors have discovered that BIO promotes the expansion of isl1+ progenitors by inhibiting GSK-3 activity.

In order to test whether BIO was capable of expanding human neonatal isl1⁺ progenitors, human neonatal CMC were isolated from the biopsies of patients with congenital heart defects into single cells and cultured for 4 days in the presence or absence of BIO. Interestingly, BIO treatment detected by immunostaining (FIGS. 2L-2P), demonstrating that wnt/β-catenin pathways have an evolutionarily conserved role in expanding isl1+ cardiovascular progenitors.

### Example 3

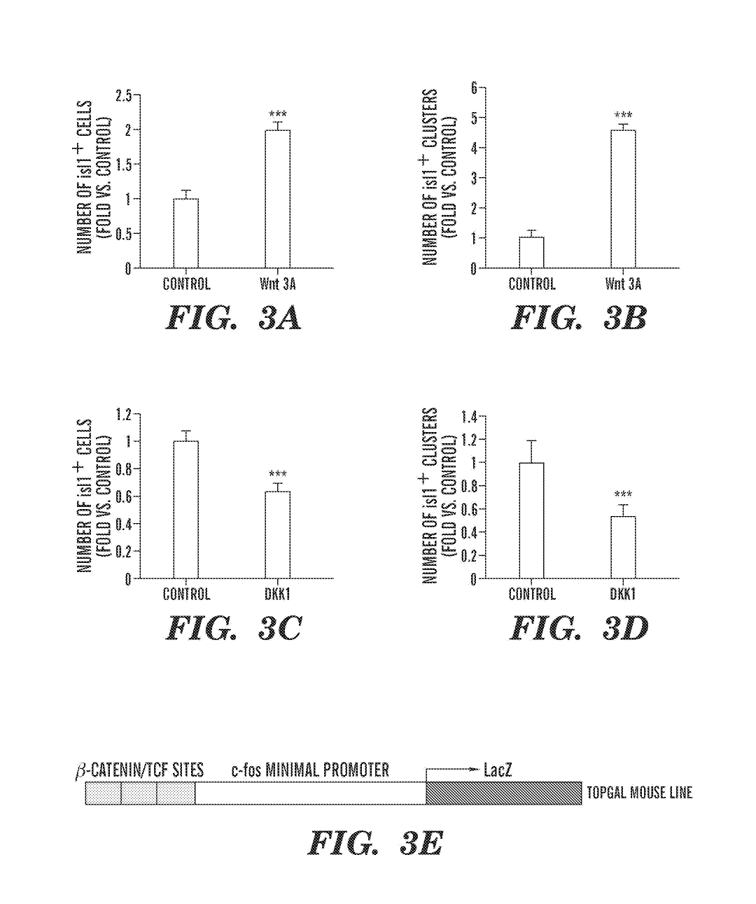

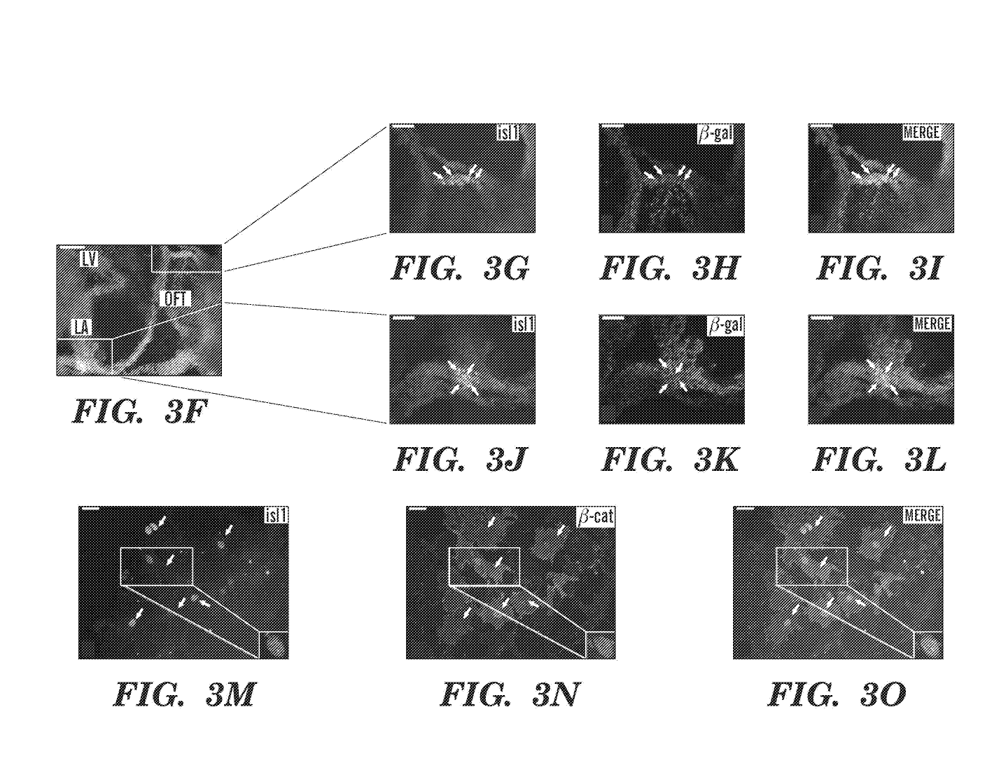

Wnt/β-Catenin Pathway Plays a Pivotal Role in the Control of Isl1+ Progenitor Expansion. The above results demonstrated that CMC-derived cues promote the expansion of isl1⁺ progenitors through the inhibition of GSK-3 activity, leading the inventors to investigate the roles of signaling molecules in the GSK-3 pathway (Dominguez and Green, 2001). The inventors examined whether Wnt3a, a well-established ligand in the Wnt/β-catenin pathway (Logan and Nusse, 2004), was able to expand postnatal isl1⁺ progenitors. Treatment with Wnt3a-conditioned medium resulted in a 2-fold increase of isl1⁺ progenitors compared with the control (FIG. 3A, p<0.001), and approximately 4-fold increase in isl1⁺ cell clusters (FIG. 3B, p<0.001) as compared to control. The inventors further cocultured CMC with a feeder layer stably secreting Wnt3a, hence providing a higher sustained level of Wnt3a activity, and observed a nearly 6-fold increase of isl1⁺ progenitors versus control (FIG. 3C, p<0.001 and approximately a 50% decrease in isl1⁺ cell clusters (FIG. 3D, p<0.001).

Cumulatively, the inventors have discovered that canonical Wnt-GSK3 signalling plays an important role in the expansion of isl1⁺ progenitors driven by CMC environment-derived cues. The inventors next performed, in isl1⁺ progenitors, in situ analysis of activated β-catenin, which is a pivotal downstream component of the canonical Wnt-Gsk3 pathway. Previous studies have established a reliable Wnt signalling indicator mouse strain, TOPGAL, which expresses β-gal under the control of a LEF/TCF and β-catenin inducible promoter (FIG. 31, DasGupta and Fuchs, 1999; Glass et al., 2005). Double immunofluoresence staining revealed that a significant population of isl1⁺ progenitors was positive for β-gal expression in the outflow tract (data not shown) and/or left atrial region (data not shown) of an ED 10.5 TOPGAL heart, suggesting that isl1+ progenitors possess active nuclear β-catenin transcriptional activity in vivo. In addition, the inventors investigated whether postnatal isl1+ progenitors had active β-catenin signalling by performing co-staining on the CMC for Isl1 and β-catenin. The inventors showed a preferential β-catenin staining in the cytoplasm of isl1⁺ progenitors (data not shown), demonstrating the onset of active Wnt signaling, which is known to inhibit the degradation of β-catenin leading to its accumulation in the cytoplasm by blocking GSK3 activity (Logan and Nusse, 2004). In some isl1⁺ progenitors, nuclear β-catenin staining was detected (data not shown), further demonstrating that postnatal isl1+ progenitors maintain active Wnt signaling. Taken together, the inventors have discovered that the wnt/β-catenin pathway plays a pivotal role in the expansion of isl1⁺ progenitors.

### Example 4

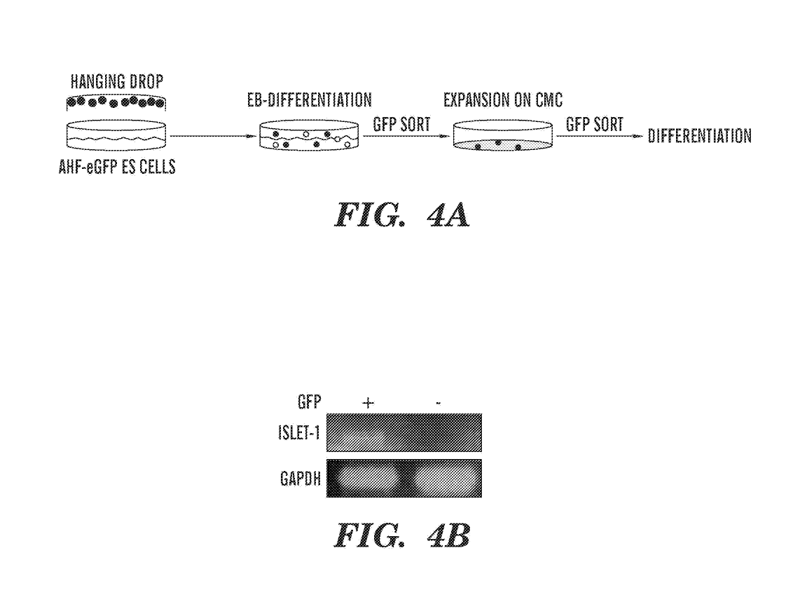

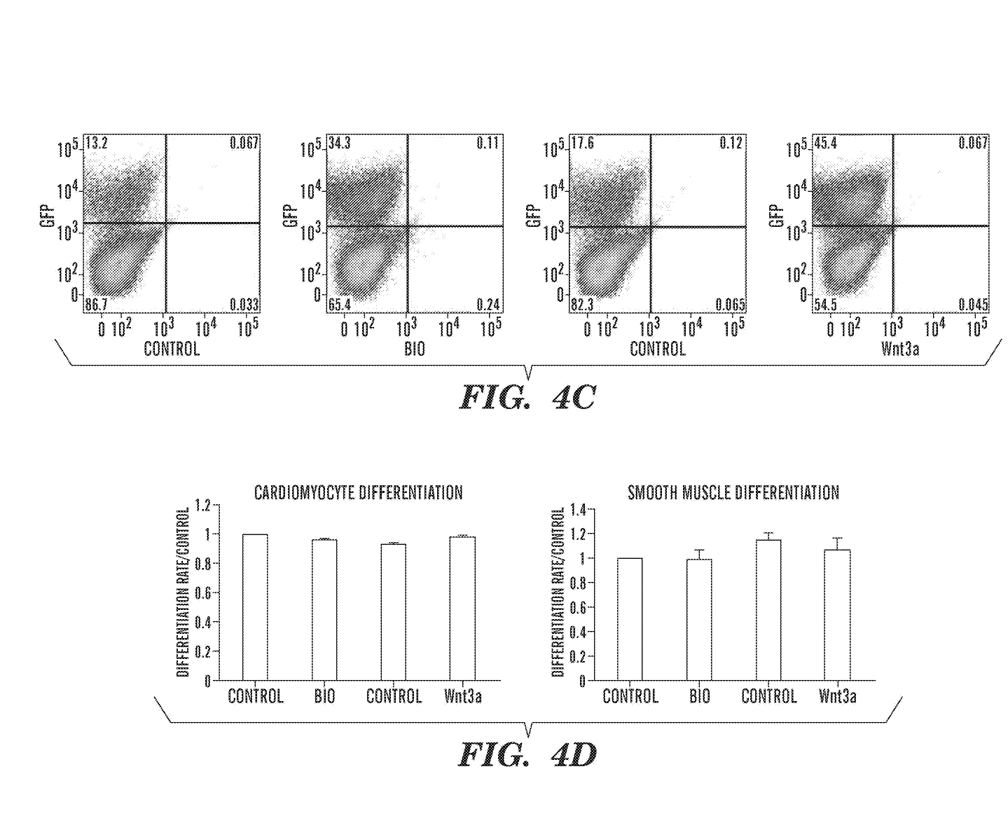

The cardiac mesenchymal cell (CMC) Feeder Layer and the Canonical Wnt Ligand Lead to a Marked Renewal of isl1⁺ Anterior Heart Field Lineage Cells. To directly determine whether Wnt ligands are secreted from the CMC to promote the renewal of isl1⁺ progenitors in a paracrine manner, the inventors developed an in vitro reconstitution of the mesenchymal niche. The inventors employed a cardiac mesenchymal feeder (CMC) layer together with fluorescence-activated cell sorting (FACS)-purified isl1⁺-enriched secondary heart lineage cells that were tagged with green fluorescent protein (GFP) under the control of a Mef2c enhancer (Dodou et al., 2003) that allowed their purification and quantification (FIG. 4A). When plated on a CMC feeder layer, FACS-purified GFP positive cells expanded and formed colonies highly enriched for Isl1 as detected by immunostaining as well as by RT-PCR (FIG. 4B, data not shown), while GFP negative cells essentially failed to form such colonies (data not shown). Furthermore, in the absence of the CMC feeder, the GFP positive cells were not able to maintain Isl1 expression (data not shown), suggesting a CMC niche-derived paracrine cue is required for the maintenance and expansion of the anterior heart field isl1⁺ progenitors.

Given that BIO enhances the proliferation of postnatal isl1⁺ progenitors, the inventors tested its ability to augment the CMC niche-driven expansion of anterior heart field isl1⁺ progenitors. As seen in FIG. 4C, there was a marked expansion of GFP positive cells with BIO treatment when compared to control. To further characterize the nature of the CMC-derived cues, the inventors tested the ability of Wnt3a to reproduce this effect. When Wnt3a conditioned media was added to the CMC, there was a significant expansion of anterior heart field GFP positive cells compared with the control (FIG. 4C).

To ensure that the multipotency of the expanded cardiac progenitors was not lost, the inventors performed differentiation studies on these cells to investigate their ability to differentiate into cardiomyocytes and smooth muscle cells. Following expansion on the CMC feeder under different conditions, the GFP positive cells were FACS purified again and were plated under differentiation conditions. As seen in FIG. 4D, both BIO— and Wnt3a-expanded progenitor cells had similar ability to differentiate into smooth muscle cells and cardiomyocytes as control-treated cells. Immunostaining of cells treated with BIO for cardiac troponin T and smooth muscle myosin heavy chain demonstrated that the canonical Wnt ligand represents a CMC-derived paracrine cue that promotes the renewal of the anterior heart field isl1⁺ progenitors whilst maintaining their multipotency (data not shown).

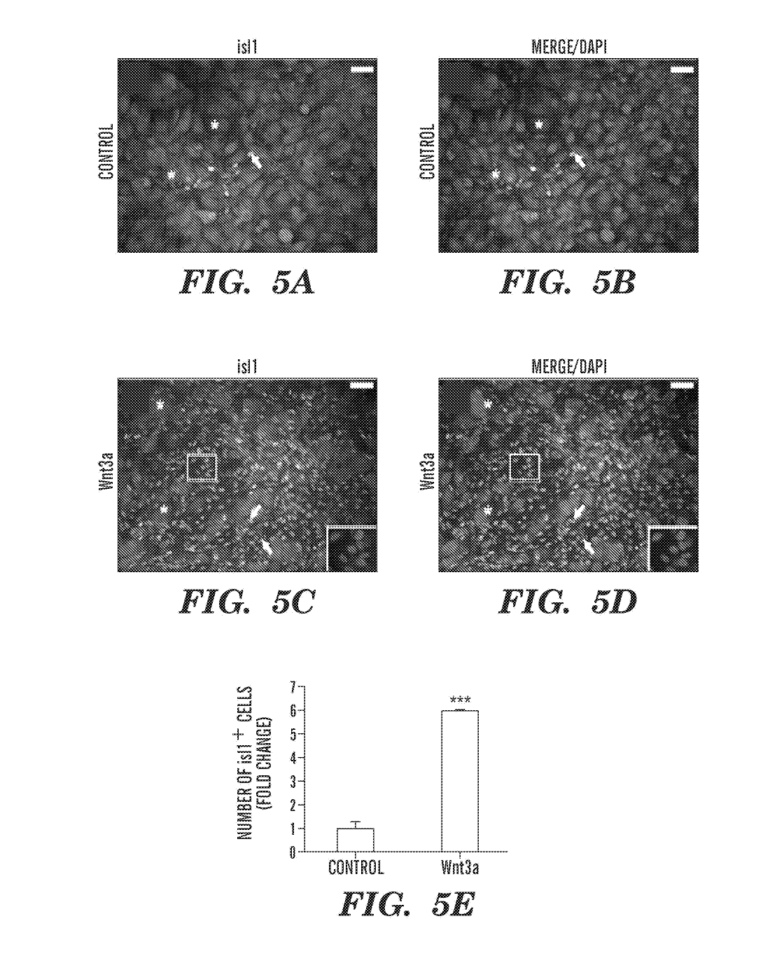

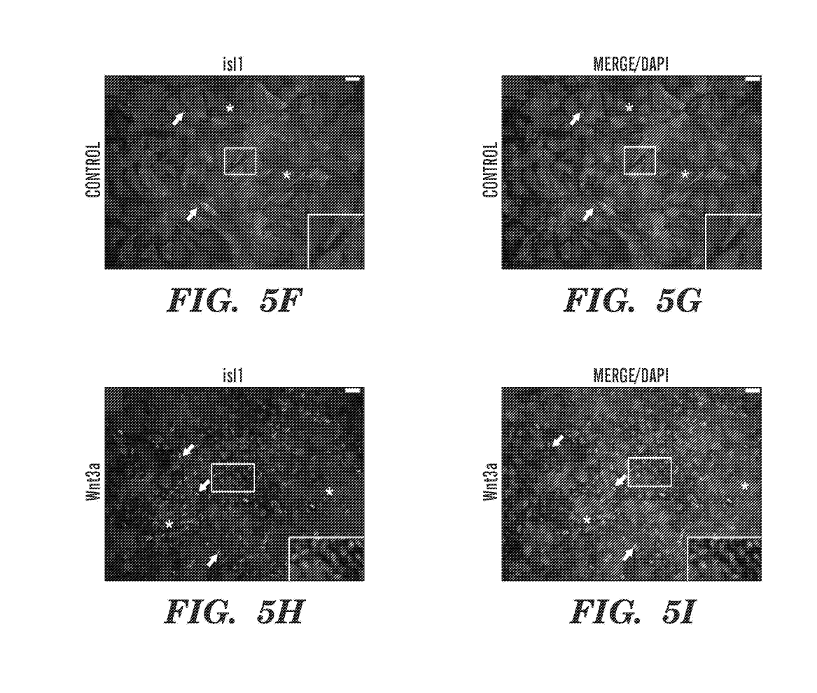

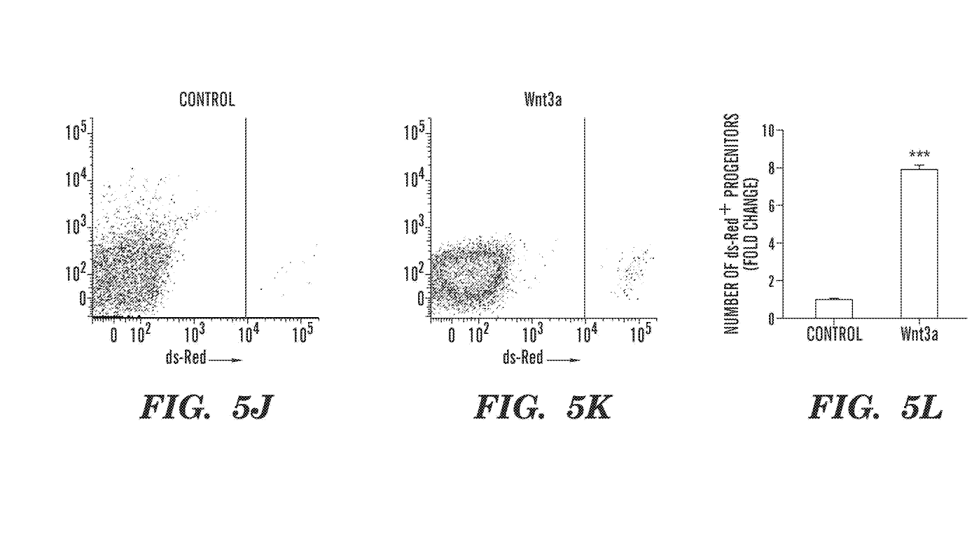

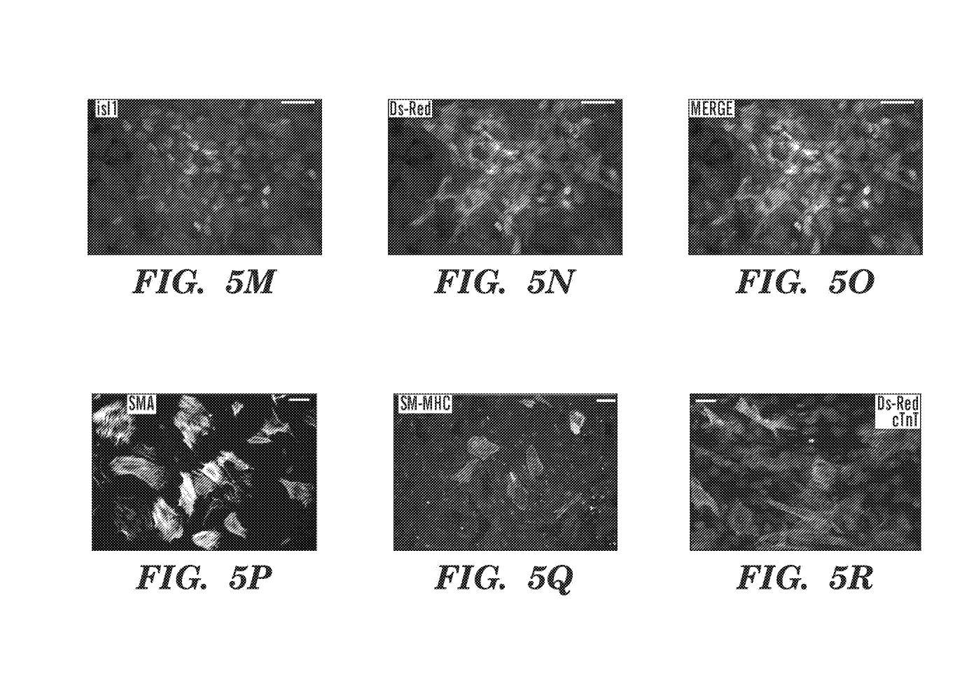

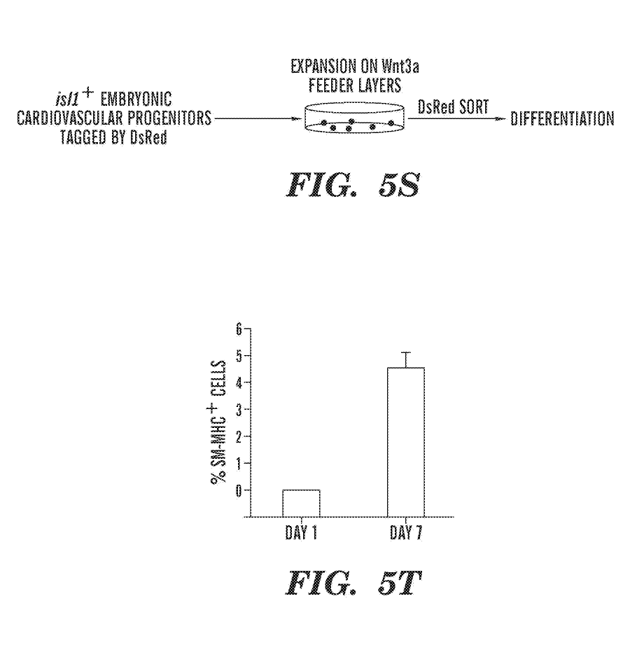

The inventors had previously shown that the CMC environment allows the expansion of embryonic isl1⁺ progenitors with maintenance of their cardiovascular potential (Moretti et al., 2006). However, the cues emanating from the CMC that promote the renewal of these progenitors remain unknown. Given the fact that Wnt3a can enhance the expansion of both postnatal and ES cell-derived anterior heart field isl1⁺ progenitors, the inventors tested whether the canonical Wnt ligand represents such a cue from CMC. As described previously (Moretti et al., 2006), anterior heart field-enriched tissues were isolated from murine embryos of approximately ED8.5, dissociated into single cells, and plated at a low density on a feeder layer consisting of a cell line stably secreting Wnt3a or its control (FIG. 5S).

### Example 5

It is shown that Wnt3a can enhance the expansion of postnatal isl1+ progenitors. Thus, the inventors tested whether this ligand also had a similar effect on the isl1⁺ embryonic progenitor subset. Single cell preparations from the secondary heart field region of approximately E8.5 embryos were generated as previously described (Moretti et al., 2006) and plated at a low density on a feeder layer consisting of a cell line stably secreting Wnt3a or its control. As shown in FIGS. 5H and 5I, the Wnt3a-secreting feeder layer triggered a marked expansion of embryonic isl1⁺ progenitors, while the control feeder essentially failed to maintain the expression of isl1 (FIGS. 5F and 5G).

To investigate the differentiation potential of these expanded embryonic isl1+ progenitors, the inventors genetically marked isl1-expressing cells by crossing isl1-IRES-Cre mice (Laugwitz et al., 2005) into the Cre reporter strain Z/RED (Vintersten et al., 2004), thereby enabling us to purify the isl1⁺ cells by flow cytometry. Cre-mediated deletion of the β-geo cassette results in the expression of Dsred under the control of a ubiquitous promoter composed of the chicken beta actin minimal promoter and the CMV immediate early enhancer, thereby enabling the inventors to purify the isl1⁺ cells by FACS. After expansion on the Wnt3a-secreting feeder layer for 7 days, Dsred-expressing cells were isolated as a distinct population by FACS analyses (FIG. 5J), which were highly enriched for Isl1 as detected by immunostaining (FIG. 5H).

After coculture on feeder layers for 7 days, dsRed-expressing cells were isolated as a distinct population by FACS analysis (FIGS. 5J and 5K). As seen in FIG. 5L, there was a significant expansion of dsRed+ cells on the Wnt3a feeder compared to the control. These dsRed+ cells were highly enriched for isl1 as confirmed by colocalization of isl1 and dsRed double immunostaining (FIGS. 5M-5O). In addition, dsRed positive cells showed essentially no expression of the cardiac marker troponin T (cTnT) or smooth muscle cell markers (a-smooth muscle actin [SMA] and smooth muscle myosin heavy chain [SM-MHC]) (data not shown). Thus the inventors have demonstrated that dsRed-expressing cells are in an undifferentiated progenitor state after expansion on Wnt3a feeder layers. When cultured in the absence of feeder layers after FACS purification, a significant proportion of dsRed+ progenitors differentiated either into smooth muscle cells (4.5±0.3%) or into cardiomyocytes (5.8±0.4%) (FIGS. 5P-5R), showing that these Wnt3a-expanded embryonic isl1+ progenitors maintain their capacity for directed differentiation.

The inventors next performed, in isl1⁺ progenitors, immunostaining analysis of activated β-catenin. Previous studies have established a reliable Wnt signaling indicator mouse strain, TOPGAL, which expresses b-gal under the control of a LEF/TCF- and β-catenin-inducible promoter (FIG. 3E, DasGupta and Fuchs, 1999). Immunostaining revealed that a significant population of isl1⁺ progenitors was positive for b-gal expression in the OFT (FIGS. 3G-3I) and/or left atrial region (FIGS. 3J-3L) of an E10.5 TOPGAL heart, suggesting that isl1+ progenitors possess active nuclear β-catenin transcriptional activity in vivo. Taken together, the inventors have demonstrated that the wnt/β-catenin pathway plays a pivotal role in the expansion of isl1+ progenitors.

### Example 6

Canonical Wnt Ligands Lead to a Marked Expansion of Isl1+ Anterior Heart Field Lineage Cells. In order to further study the effect of wnt/β-catenin on the renewal and differentiation of isl1⁺ progenitors, the inventors established an ES cell system to provide a reliable source of purified cardiac progenitor cells. The inventors initially generated an ES cell line in which eGFP was targeted to the genomic isl1 locus, but this system proved suboptimal as the GFP signal was not strong enough for FACS detection. As a result, the inventor used a Mef2c.

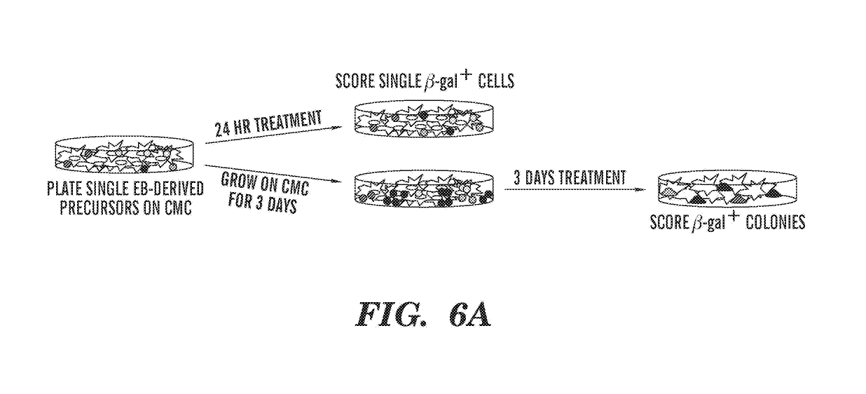

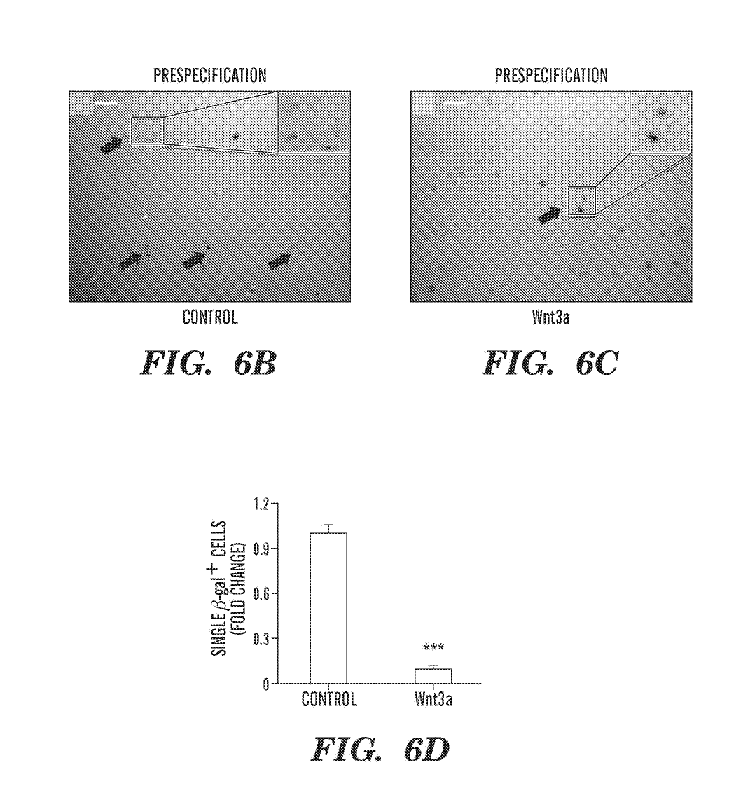

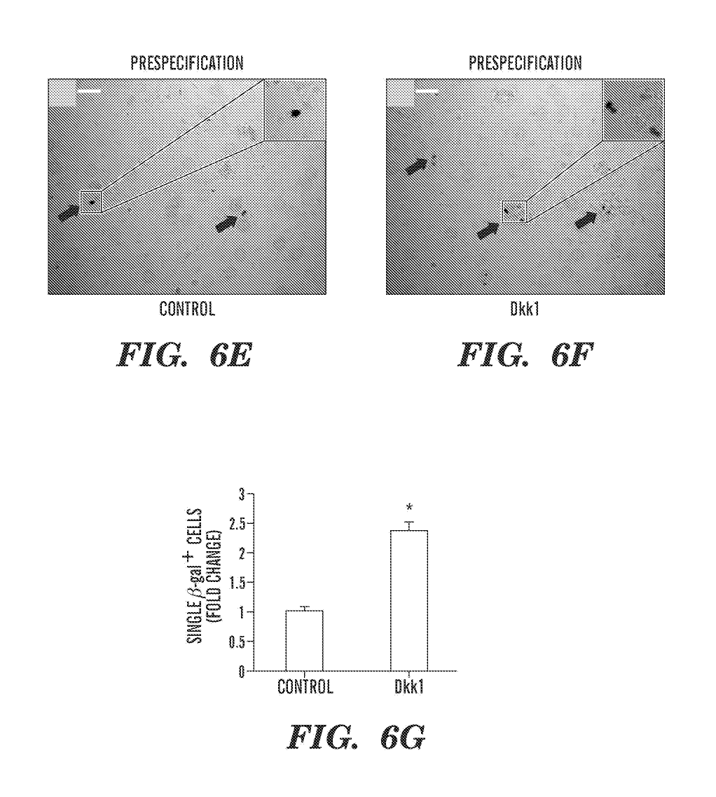

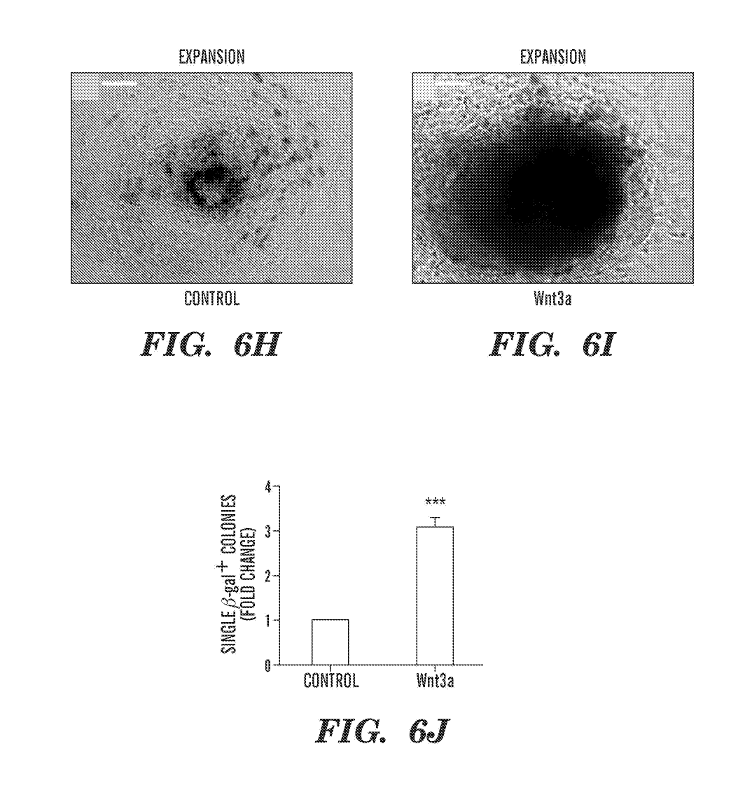

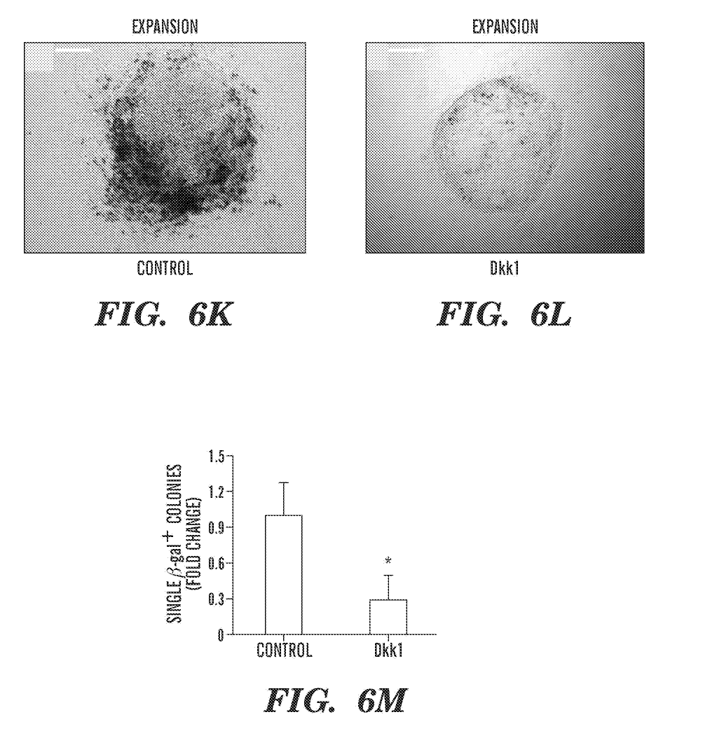

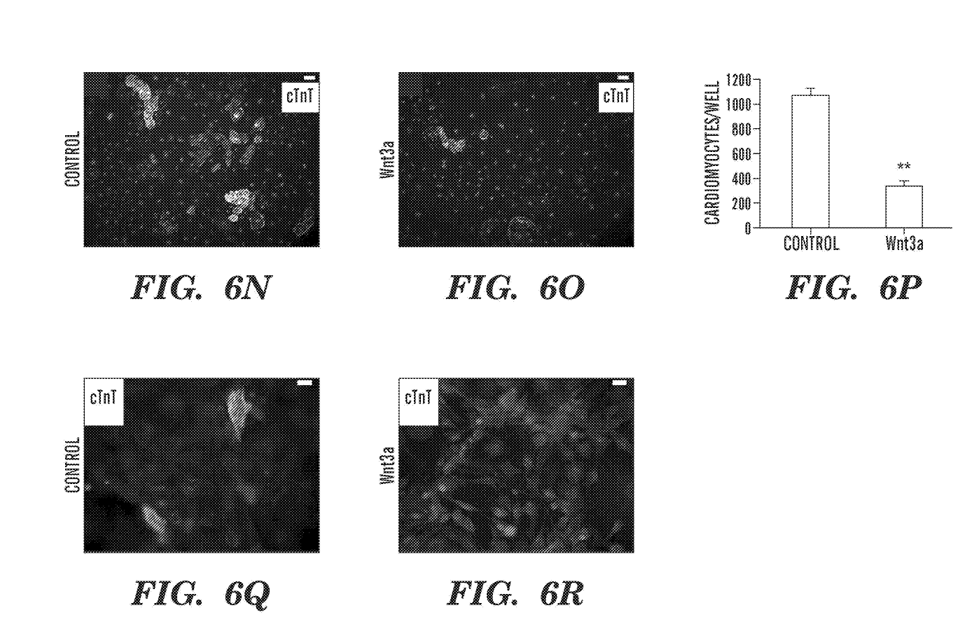

Mef2c is a direct downstream target of isl1, and an enhancer/promoter of this gene has been recently shown to be specifically expressed within the isl1 domain of the anterior heart field (AHF) (Dodou et al., 2004). Within the minimally essential region of this enhancer/promoter two isl1 binding sites were identified (FIG. 6A), and point mutations in these sites completely abrogated its expression, showing the requirement of isl1 expression for this enhancer/promoter to function (Dodou et al., 2004).

The AHF enhancer/promoter (kindly provided by Dr. Brian Black, UCSF) was used to generate a transgenic mouse line that showed a GFP expression pattern that was completely restricted to the AHF and its derivatives, identical to that previously described (FIG. 6A and Dodou et al., 2004). ES cell lines were derived from these transgenic mice. Following differentiation, these ES cell lines showed areas of strong GFP expression by embryoid body (EB) day 5 to 6, and by EB day 10, the majority of GFP⁺ areas were beating. FIG. 3B shows the FACS profile of EB day 6 differentiated ES cells. When the GFP+ cells were sorted and plated onto fibronectin-coated slides, they demonstrated the ability to spontaneously differentiate into cardiomyocytes and smooth muscle cells (FIGS. 6C and 6D). To confirm the AHF identity of the GFP+ cells, the inventors measured isl1 and mef2c expression in freshly sorted GFP+ cells from EB day 6. As seen in FIG. 6E, there was a significant enrichment of isl1 and mef2c message in the GFP+ compared to the GFP− population.

To test the ability of wnt/β-catenin signals to stimulate the expansion of the ES-derived cardiac progenitors, freshly sorted AHF-GFP+ cells were directly plated onto control cells or cells stably secreting Wnt3a for 7 days. As seen in FIG. 6F, there was a significant enrichment of isl1 expression in GFP+ cells plated on the Wnt3a feeder layer compared with GFP+ cells plated on the control layer. This observation was further confirmed by isl1 immunostaining (data not shown).

The inventors next performed studies on isl1⁺ AHF lineage cells to investigate their ability to differentiate into cardiomyocytes and smooth muscle cells following their expansion by Wnt3a or BIO. Wnt3a or BIO-expanded progenitor cells had similar ability to differentiate into both cell lineages as control treated cells.

### Example 7

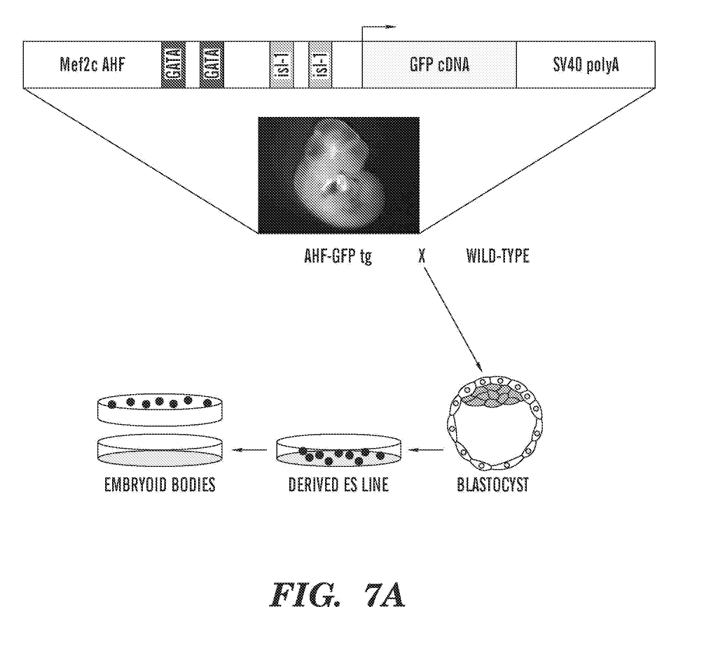

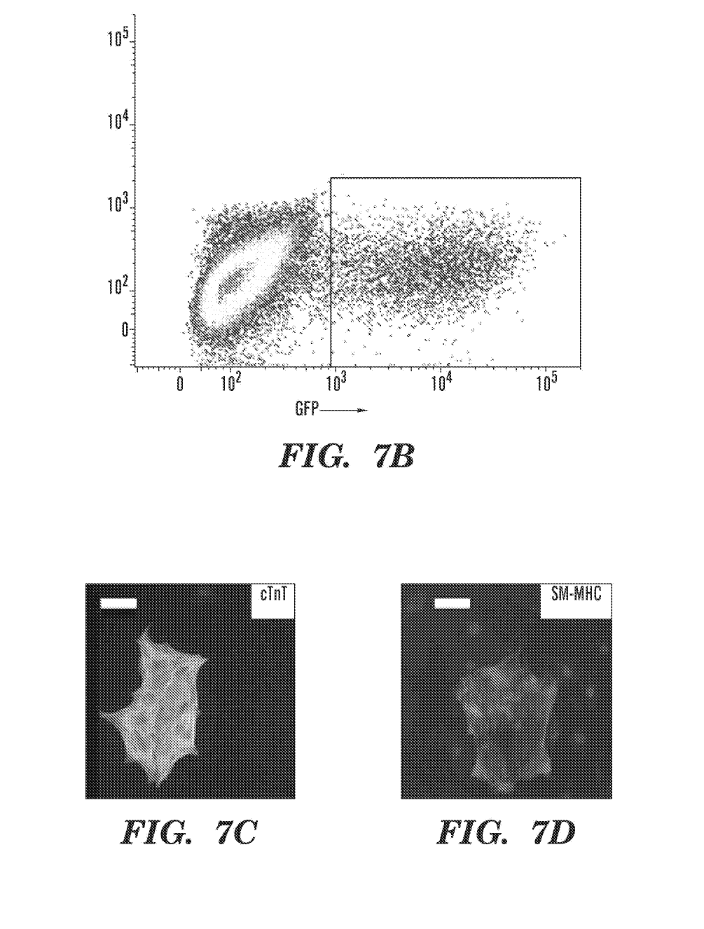

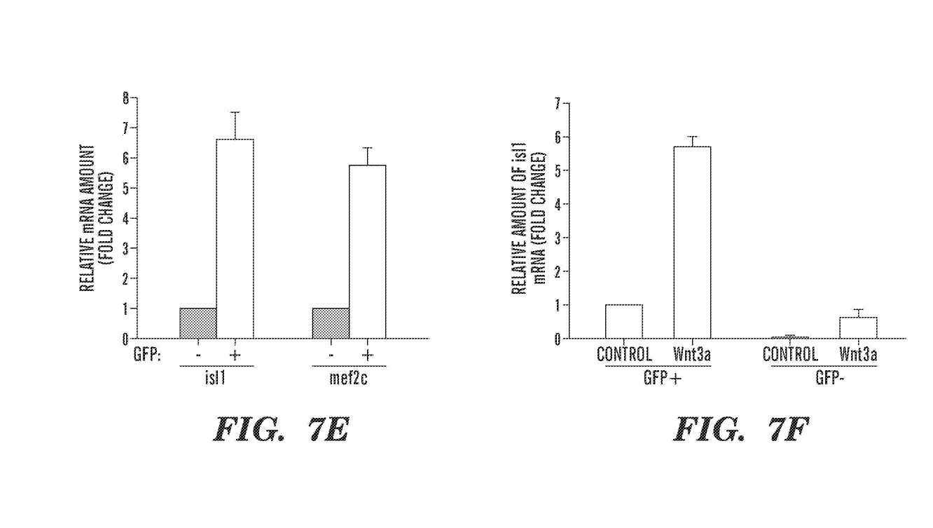

The wnt/β-Catenin Pathway Regulates the Prespecification, Expansion, and Differentiation of Isl1+ Cardiovascular Progenitors. The inventors next examined whether Wnt signals are capable of augmenting the initial number of ES cell-derived isl1⁺ clones. In order to do this, the inventors used a previously described isl1-nlacZ knockin ES cell line (Moretti et al., 2006). After 4.5 days of differentiation, EBs were dissociated into single cells and plated at low density on a CMC feeder layer. To score the effect of the CMC feeder on the prespecification of mesodermal precursors toward MICPs, the inventors quantified the single b-gal⁺ cells 24 hr after treatment with various reagents (FIG. 7A). The inventors showed that the addition of Wnt3a-conditioned medium resulted in a marked inhibition in the formation of MICPs (FIGS. 7B-7D), demonstrating that canonical Wnt ligands from CMC have an inhibitory effect on this step. In order to investigate whether inhibition of the Wnt signal leads to a higher rate of prespecification, the inventors tested the effect of Dkk1-conditioned medium finding a significant increase of single β-gal⁺ cells(FIGS. 7E-7G).

The inventors have demonstrated that the CMC feeder layer utilizes a Wnt/β-catenin pathway to carefully titrate the number of MICPs via a negative regulatory pathway that inhibits pre-specification, a result that is consistent with previous studies in other systems that have demonstrated that the Wnt/β-catenin pathway can markedly inhibit cardiogenesis (Marvin et al., 2001; Schneider and Mercola, 2001; Tzahor and Lassar, 2001).

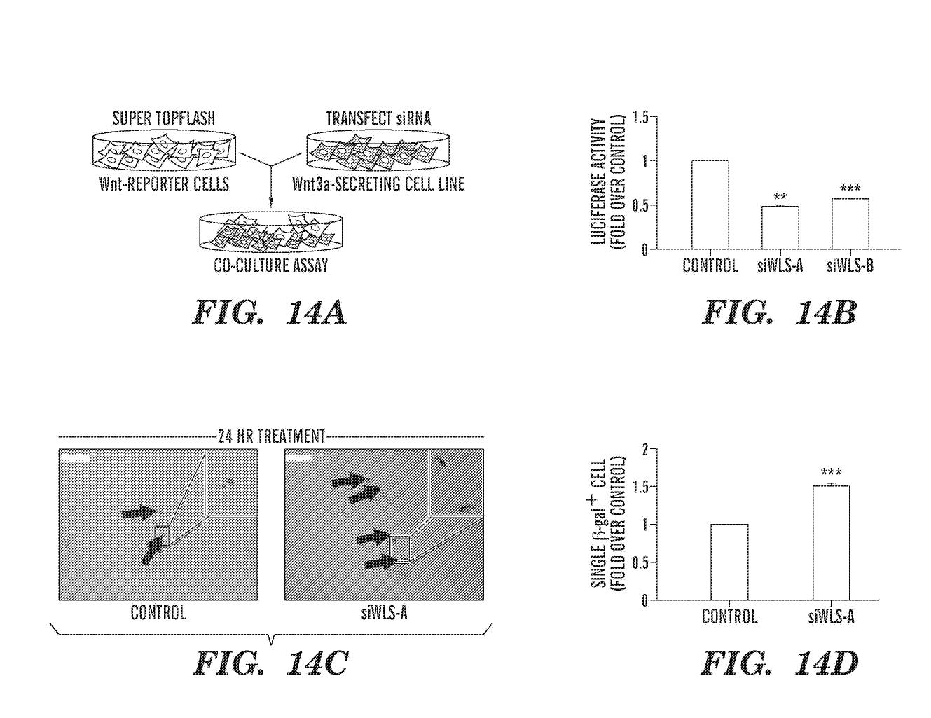

The inventors confirmed this effect by blocking the secretion of Wnt ligands from the CMC. One component of the Wnt pathway, Wls/Evi, has been identified to be required for the secretion of Wnt in Wnt-producing cells (Banziger et al., 2006; Bartscherer et al., 2006). The inventors therefore designed two siRNAs against murine Wls/Evi, siWLS-A (SEQ ID NO:1) and siWLS-B (SEQ ID NO:2) to knock-down its expression. To test the efficacy of these siRNAs, the inventors transfected them into a Wnt3a-producing cell line and then cocultured these transfected cells with a Wnt reporter cell line harboring the TCF/Lef reporter construct superTOPFLASH (FIG. 14A). Both siRNAs caused a significant reduction of luciferase activity over control (FIG. 14B), demonstrating their ability to knock down the expression of the endogenous WLS gene. The inventors next infected the CMC feeder layers with siRNAs against WLS and observed a significant augmentation of the number of single β-gal⁺ cells (FIGS. 14C and 14D). Cumulatively, these results demonstrate that the CMC niche utilizes a paracrine wnt/β-catenin pathway to carefully titrate the number of MICPs via a negative regulatory pathway that inhibits prespecification.

As shown herein that the Wnt/β-catenin pathway can expand a hierarchy of isl1⁺ progenitors, the inventors assessed if committed to MICPs, the CMC-derived Wnt cues may promote the expansion of these prespecified cardiovascular progenitors. To investigate this, the inventors cocultured mesodermal precursors arising from isl1-nlacZ knockin ES cells with the CMC feeder layers for 3 days, during which the feeder cells presumably prespecified a substantial number of mesodermal precursors toward MICPs. The inventors then added either control- or Wnt3a-conditioned media and allowed the coculture to proceed for another 3 days, and scored the effect on promoting the expansion of these prespecified MICPs by comparing the size and homogeneity of β-gal⁺ colonies. The inventors showed that the addition of Wnt3a-conditioned medium resulted in the formation of markedly expanded and relatively homogeneous β-gal⁺ colonies (FIG. 7I). In contrast, treatment with control-conditioned medium produced colonies that generally had a significantly sparser distribution of β-gal⁺ cells (FIG. 7H). FIG. 7J shows the quantitative effect of Wnt3a treatment versus control. To test whether the canonical Wnt signal is required for the expansion of prespecified MICPs, the inventors partially blocked the Wnt pathway with Dkk1-conditioned media. While the control-conditioned medium allowed a basal level of expansion of MICPs (FIG. 7K), Dkk1 caused a marked reduction of the expansion of the committed MICPs with primarily single β-gal⁺ cells distributed within the colony (FIGS. 7L and 7M).

The inventors next examined whether the wnt/β-catenin pathway regulates the differentiation of isl1⁺ cardiovascular progenitors. In order to obtain a purified population of cardiac progenitors to perform these studies, the inventors used freshly sorted AHF-GFP⁺ cells from day 6 EBs as described in the previous section (FIG. 6A). These cells were directly plated onto fibronectin-coated slides and allowed to undergo spontaneous differentiation. The presence of Wnt3a-conditioned media resulted in a significant decrease of differentiated cardiomyocytes as compared with control media (FIGS. 7N-7P), even though the total cell number in both samples was comparable (data not shown). Consistent with this observation, when AHF-GFP⁺ cells were cocultured on a Wnt3a-secreting feeder layer, cardiomyocyte differentiation was completely abrogated compared to that on the control feeder (FIGS. 7Q and 7R). Taken together, the inventors have discovered a triphasic wnt/β-catenin paradigm that represents a major component of the molecular mechanism by which each specific step, prespecification, renewal, and subsequent differentiation is differentially regulated during cardiogenesis.

### Example 8

Expression of a Stabilized Form of β-catenin AHF Lineage Cells In Vivo Leads to a Markedly Expanded Isl1+ Second Heart Field and Negatively Regulates the Differentiation of Isl1+ Progenitors in OFT. To unravel the effects of wnt/β-catenin on the renewal and differentiation of isl1⁺ cardiovascular progenitors in vivo, the inventors examined the consequences of constitutively activating β-catenin in the isl1⁺ progenitors and their derivatives in the AHF lineage cells. Previous studies have established that various serine/threonine residues located in the exon3 of β-catenin are the targets of phosphorylation of GSK-3 and deletion of exon3 prevents this phosphorylation and subsequent degradation of β-catenin, thereby generating a stabilized form (Logan and Nusse, 2004). A mouse strain in which exon3 of β-catenin is flanked by loxP sites was generated previously (Catnb^{+/lox(ex3)}, Harada et al., 1999).

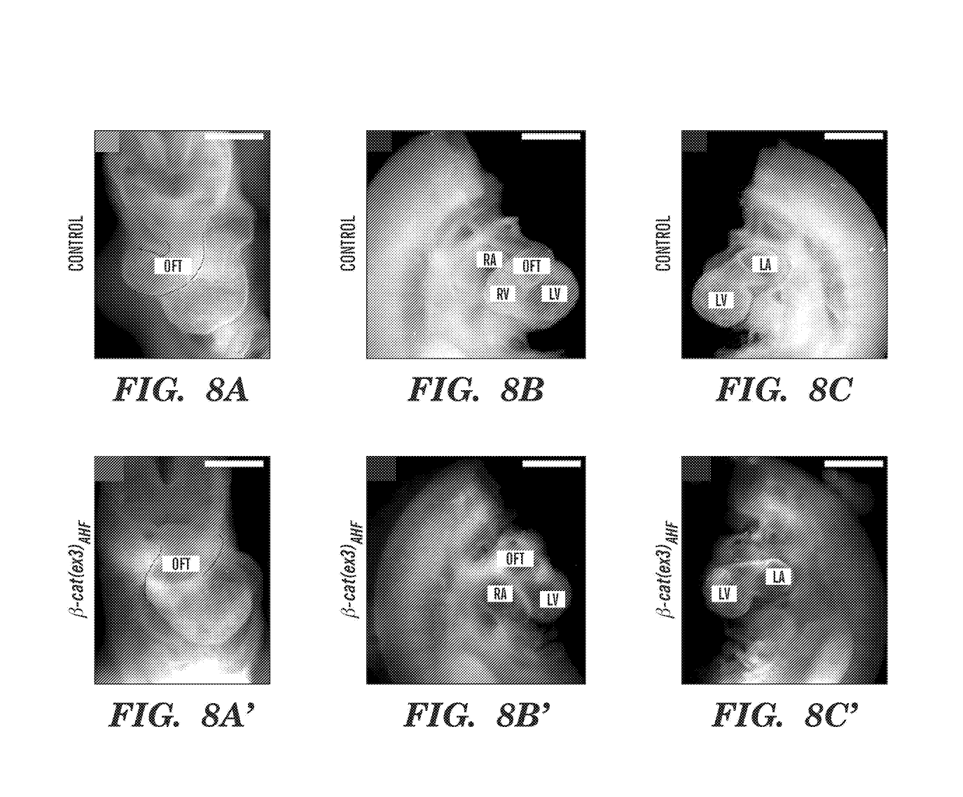

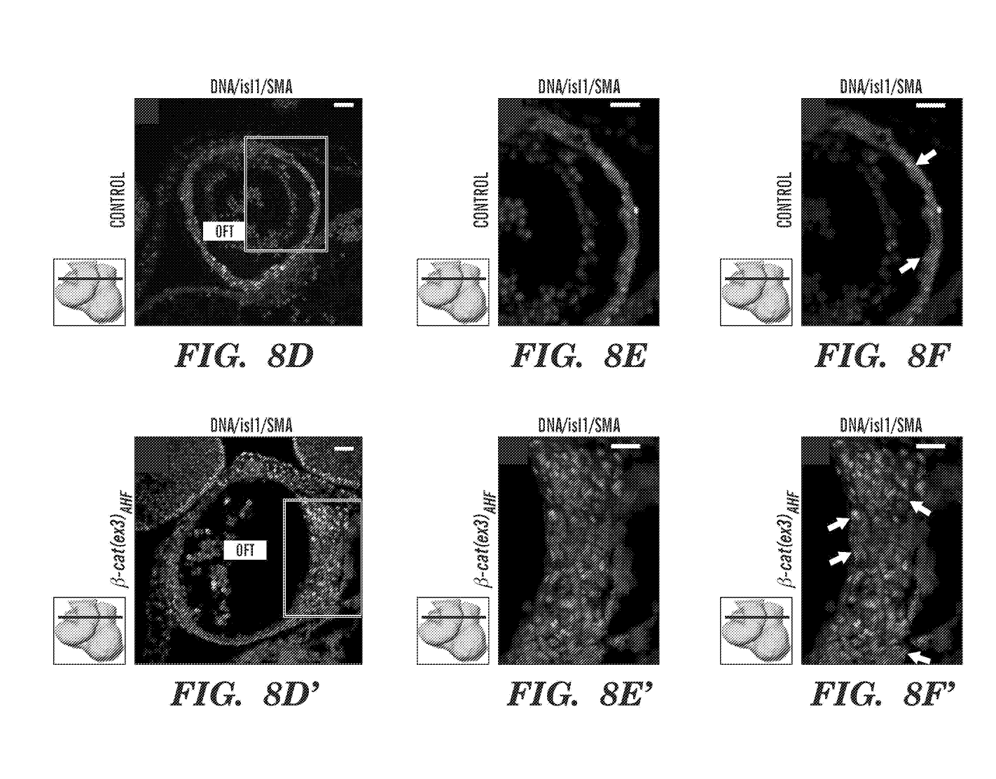

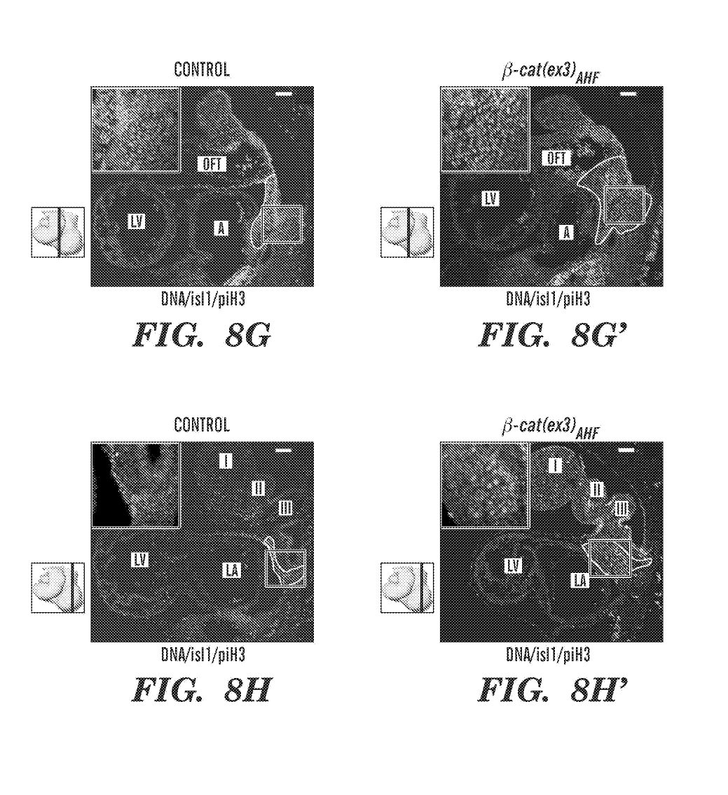

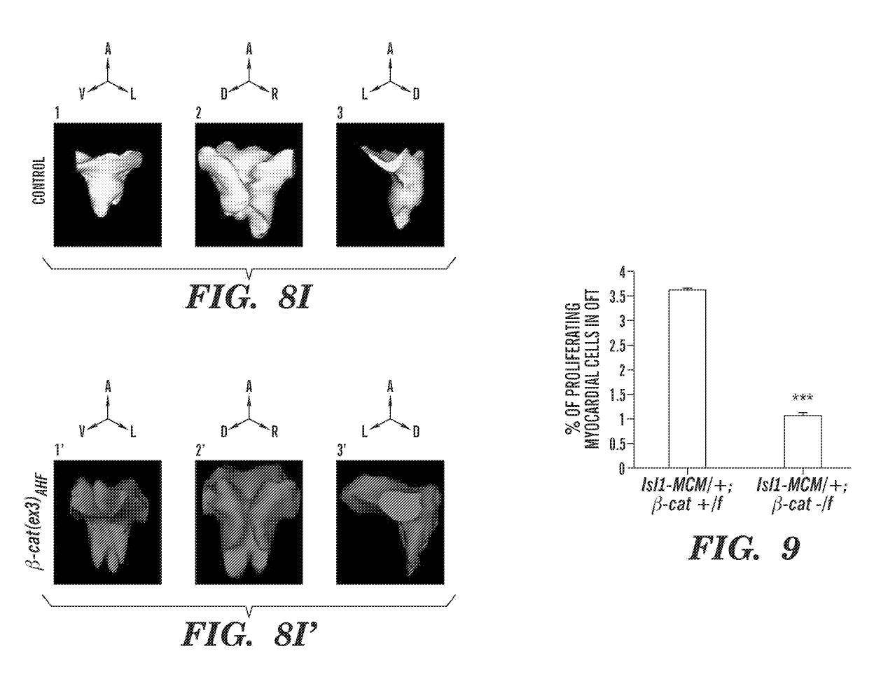

The inventors used a transgenic mef2c-AHF-Cre mouse line, in which the Cre expression is controlled by an enhancer/promoter region in the mef2c gene that exclusively directs expression to the AHF and its derivatives, and is dependent on isl1 for its expression (Verzi et al., 2005; Dodou et al., 2004). Catnb^{+/lox(ex3)} mice were crossed with mef2c-AHF-Cre line to generate double heterozygous mef2c-AHF-Cre; Catnb^{+/lox(ex3)} embryos (hereafter referred to as β-cat[ex3]_{AHF}), in which Cre-mediated removal of exon3 in the β-catenin gene results in the production of a stabilized and constitutively active molecule specifically in the AHF. The inventors analyzed E9.5 embryos, because the AHF and its derivatives give rise to recognizable cardiac structures at this time. As shown in FIGS. 8A-5C′, while the primary atrium and left ventricle looked essentially normal in β-cat[ex3]_{AHF} embryos, the OFT appeared to have a morphogenic defect characterized by a marked dilation, with a larger cross-sectional diameter, and truncated length when compared with somite-matched controls, a defect that appeared with complete penetrance (4/4). In addition, the mutants failed to exhibit a distinct right ventricular structure, which was readily appreciable in the control embryos. The rest of the embryonic structures appear normal in the mutants compared to controls (data not shown).

To further study the OFT abnormalities in β-cat[ex3]_{AHF} embryos, the inventors performed coimmunostaining on sections with antibodies for isl1 and SMA, a marker for embryonic myocardium (Xu et al., 2004; Sun et al., 2007). Consistent with the morphological defects observed in whole mount embryos (FIGS. 8A-8C′), sections of the mutants showed a relatively larger OFT with a discontinuous immunoreaction for SMA across the myocardial layer of the OFT, while the control sections maintained uninterrupted signals (FIGS. 8D-8F′ and data not shown). All the isl1-expressing cells in the myocardial layer of control OFT also coexpressed SMA (FIG. 8F and data not shown), in agreement with a previous study (Sun et al., 2007), and there are a considerable number of isl1-expressing cells negative for SMA in the mutant OFT “myocardial” layer (FIG. 8F′ and data not shown). Given that cardiac progenitor cells from the AHF express cardiomyocytic markers once they migrate into the OFT (Waldo et al., 2001), lack of SMA expression in the isl1-expressing cells in the mutant demonstrates that a gain of function of β-catenin in the isl1+ AHF progenitors inhibits their differentiation in the OFT, which is shown in the inventors in vitro results showing inhibition of the differentiation of isl1+ cardiac progenitors by canonical Wnt signals (FIGS. 7N-7R).

The inventors next examined the effect of cell-autonomous changes of the canonical Wnt pathway in the isl1⁺AHF in E9.5 β-cat[ex3]_{AHF} embryos. Previous studies have established that a substantial portion of the AHF is composed of the pharyngeal mesoderm between the OFT and the inflow tract (IFT) of the early embryonic heart and that isl1-expressing cells mark a substantial amount of AHF lineage (Waldo et al., 2001; Cai et al., 2003). Immunostaining on sagittal sections of E9.5 embryos revealed that the isl1⁺ pharyngeal mesodermal cells, as outlined by the orange dashed line in FIGS. 8G-8H′, appeared to be markedly expanded in the β-cat[ex3]_{AHF} embryo compared to that in the somite-matched litter-mate control in both medial (FIGS. 8G and 8G′) and lateral (FIGS. 8H and 8H′) regions. 3D reconstruction from serial sections was next performed to better appreciate the effect of the gain of function of β-catenin on the expansion of the isl1⁺ AHF. Consistent with the results from the representative lateral and medial sections, the isl1⁺ pharyngeal mesoderm between the OFT and the IFT was significantly enlarged in the mutant compared to the control (FIGS. 8I and 8I′).

To test whether the expansion of the AHF in the mutant was associated with an increased proliferation of isl1-expressing cells, the inventors counted cells double stained for isl1 and the mitotic marker, phosphorylated histone H3 (pi-H3). The proportion of pi-H3 and isl1 double positive cells in the AHF from the average of two E9.5 mutant embryos was 15.8%, which was significantly higher than that seen in control embryos (9.0%, p<0.01, c 2 test). In contrast, there was no appreciable difference in the proliferation rate of neuroepithelial cells between mutants (11.6%) and controls (10.6%, p=0.42).

### Example 9

Decreased Proliferation of the OFT Myocardial Cells in Murine Embryos with a Temporally Controlled Loss of Function of β-Catenin. The inventors next performed loss-of-function experiments as shown in FIG. 9. The inventors crossed double heterozygous isl1-MCM⁺; β-catenin^{+/-→}mice with β-catenin floxed homozygous mice to obtain isl1-MCM^{+/-; β-cat}^{-/f} mutants and isl1-MCM^{+/-}; β-cat^{+/f} controls. Tamoxifen was injected into pregnant females at E9.5, and embryos were harvested at E11.5. Pi-H3 immunostaining showed a markedly decreased proliferation rate of myocardial cells in the OFT of the mutant when compared to control embryos (data not shown). As myocardial cells in the OFT are primarily derived from isl1⁺ secondary heart field progenitors (Cai et al., 2003), the results herein demonstrate that β-catenin plays an important role in the proliferation of isl1⁺ lineage cells.

### Example 10

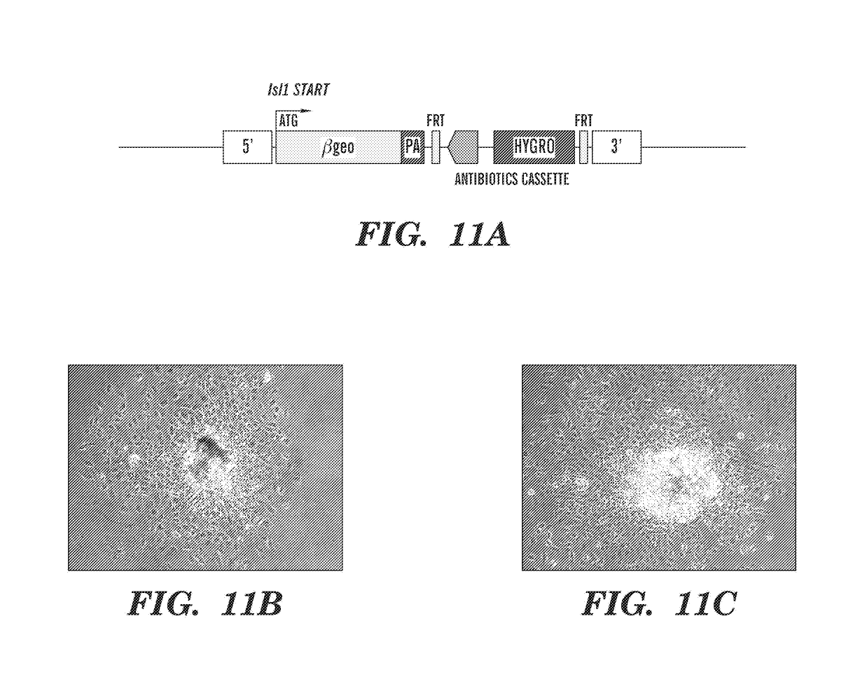

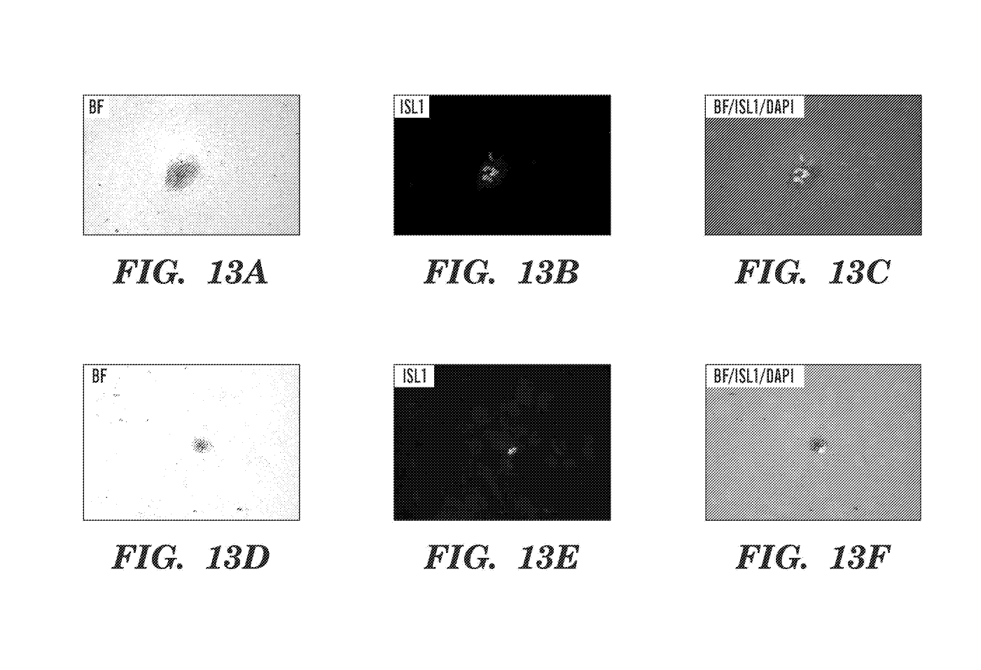

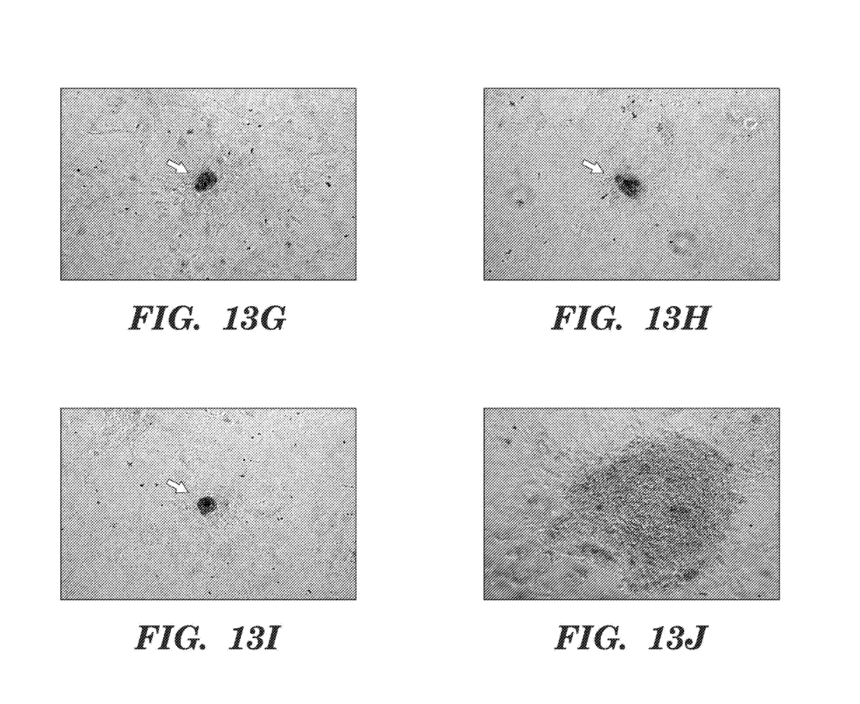

Human ES cells induced to enter islet 1+ lineage by inhibition of wnt/β-catenin pathway. In brief, in order to establish an induction of hES cells along islet 1+ lineages, and their subsequent renewal, the inventors used Isl1-βgeo BAC transgenic hES cell lines as a source. When allowed to differentiate in culture, hES cells generate embryoid bodies (EBs) that contain a broad spectrum of cell types representing derivatives of the three germ layers. The inventors analyzed the time course of Isl1 expression in developing EBs from Isl1-βgeo BAC transgenic hES cells by RT-PCR and β-gal staining. In undifferentiated ES cells and early EBs, Isl1 expression was not detected on mRNA and protein level. Within 4 to 6 days of EB differentiation, ES cell derived progenitors expressing Isl1 arose, as demonstrated by transcript detection and β-gal activity (FIGS. 11B and 11C). Immunohistochemistry using a monoclonal anti-Isl1 antibody revealed co-expression of Isl1 and β-gal proteins, indicating that Isl1 gene expression can be monitored by LacZ staining (FIGS. 13A-13F).

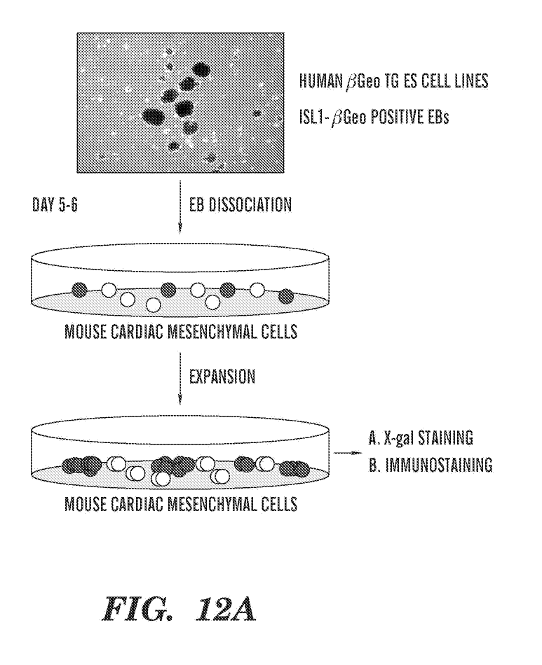

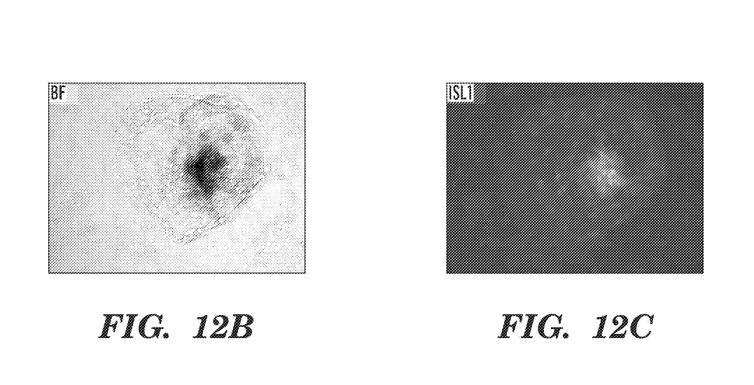

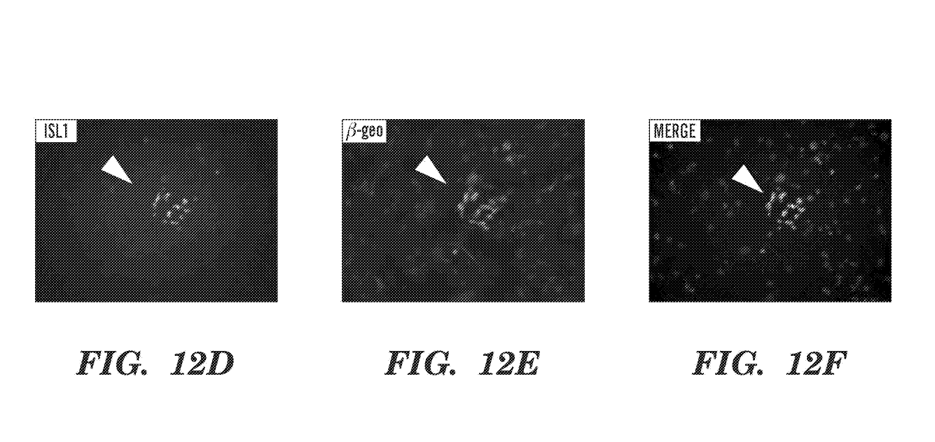

To test whether the mesenchyme environment could support expansion of Isl1+ cardiac precursors arising during hES cells differentiation, the inventors dissociated human EBs from Isl1-βgeo BAC transgenic hES cells at day 5 or 6 into single cells and plated them at low density on feeder layers of murine cardiac mesenchymal cells (CMC). After 1 or 2 days, the inventors showed single or dividing β-gal+ cells in the CMC co-culture (FIGS. 13A-13F), but none were detected on other surfaces. Within 5 days, clones with a distinct morphology were visible exclusively on top of the CMC feeders, and around 10±5% presented β-gal activity in a characteristic focal pattern, reflecting that the clones originated from a single expanding β-gal+ cell (FIGS. 12A, 12B-12F and 13A-13F). Mock treatment by plating dissociated cells from day 5 EBs on other surfaces resulted in attachment and survival of a small number of cells without any clone formation.

### Example 11

High-Throughput Chemical Screening and the Identification of Key Steps in Cardiovascular Cell Lineage Diversification. In the current study, the inventors employed high-throughput screening to identify a series of compounds that can trigger renewal of the postnatal isl1⁺ progenitors. The ability to reconstitute the CMC niche with FACS-purified isl1⁺ cardiovascular progenitors derived from murine ES cells enables the development of new chemical screens to identify additional renewal signals for isl1⁺ progenitors, and pathways that drive their differentiation into cardiac, smooth muscle, and endothelial cellular progeny, as well as a method to identify specific agents that might drive the directed differentiation of MICPs into coronary arterial, cardiac muscle, and pacemaker lineages. This ultimately enables large scale engineering of certain heart tissue components for clinical therapeutic use and research studies.

CMC and the Microenvironmental Cues for the Renewal of a Hierarchy of Isl1+ Cardiovascular Lineages. One of the key steps in amplifying isl1⁺ cardiovascular progenitors as demonstrated herein was the ability to expand the rare pool of these progenitors on CMC feeder layers derived from the neonatal and embryonic heart. This feeder layer allowed the renewal of isl1⁺ cardiovascular progenitors with the maintenance of their multipotentiality. Because these cells are normally found in the embryonic and postnatal heart, the possibility exists that the CMC act as the in vivo microenvironment that serves to inhibit differentiation, activate their expansion, and maintain their multipotency. The inventors demonstrated that there was a preferential localization of clusters of isl1⁺ cells in the neonatal mouse (data not shown) and human heart (data not shown) in an in vivo microenvironment of surrounding nonmyocytic CMC that serve as an insulator from triggers of cellular differentiation. Because isl1⁺ progenitors were discovered to be largely localized in the secondary heart field and migrate into a region of differentiating cardiac cells in the primordial heart tube, the inventors demonstrate that the isl1⁺ cardiovascular progenitors first encounter this microenvironment early during the course of cardiogenesis, and that it plays a critical role in the maintenance of the multipotency of these precursors that are destined to form distinct cell lineages in discrete regions of the heart.

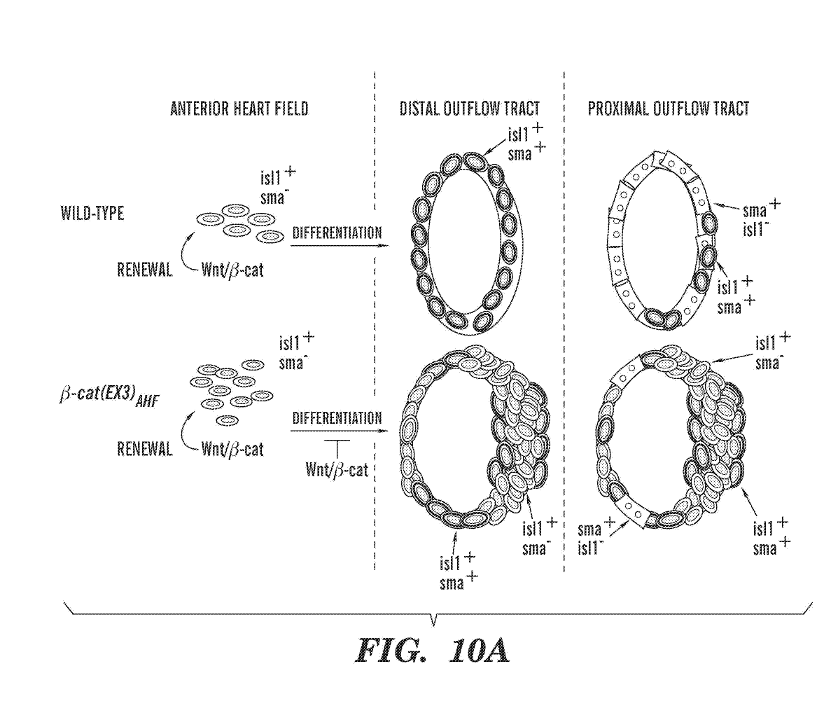

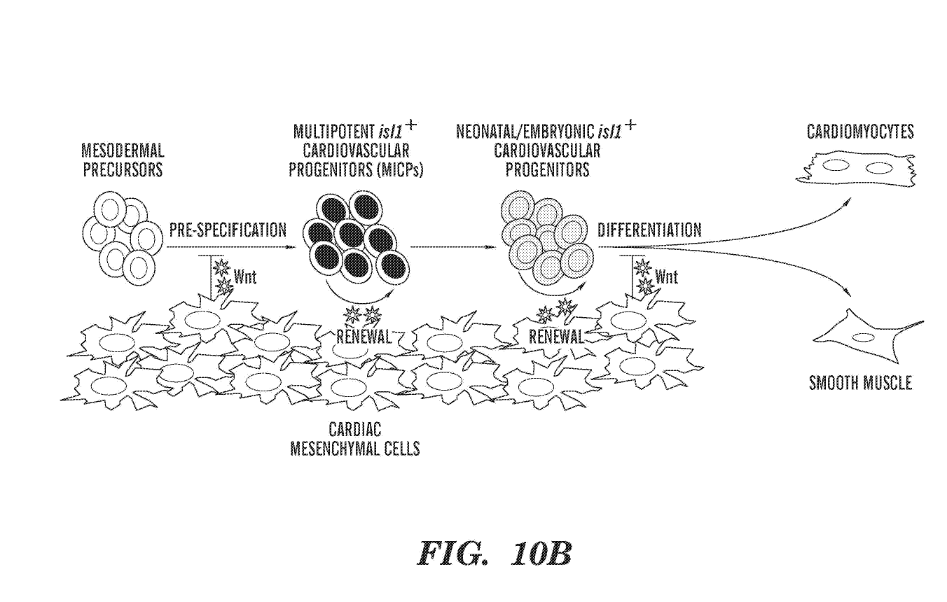

Canonical Wnt Signals Are a Major Component of the CMC Microenvironment that Controls the Renewal of a Hierarchy of Isl1+ Cardiovascular Progenitors. Through the use of chemical screening and a panel of gain- and loss-of-function studies, the inventors show that the effects of canonical Wnt ligands is sufficient to renew the hierarchy of isl1⁺ cardiovascular progenitors, as demonstrated by studies on postnatal, embryonic, and ES cell systems. Thus, the inventors have discovered a beginning of the molecular pathways of the microenvironmental niche which regulates the hierarchy of isl1⁺ cardiovascular progenitors. While previous studies have established a role for canonical Wnt signals in cardiac specification in ES cells, there has been significant controversy, as two studies proposed a positive role of Wnts in this function (Nakamura et al., 2003; Naito et al., 2006) while another suggested the opposite (Liu et al., 2007). As such, it has proven difficult to precisely pinpoint the exact molecular mechanism by which Wnt ligands might exert control on the complex process of cardiogenesis. The inventors have demonstrated herein, utilizing FACS-purified embryonic and ES cell-derived cardiovascular progenitors, that Wnt signals emanating from the CMC play a major role in cardiogenesis. The inventors have demonstrated, with a level of resolution not previously shown, that Wnt signaling has a significant role in the fate of specific subsets of isl1⁺ progenitor cell lineages. The inventors have discovered that positive Wnt signalling induces mesodermal precursors to give rise to MICPs, whereas negative wnt signalling results in the MICP and bipotent precursors to activate renewal, and the transitional isl1+/sma+ cells in the myocardium of the OFT, where it inhibits differentiation (FIG. 10B). Thus, the inventors have discovered a complex Wnt signaling within cardiogenesis. In this regard, the inventors have demonstrated the in vivo constitutive activation of β-catenin pathways within isl1⁺ AHF progenitors results in their massive accumulation, near complete inhibition of myocytic differentiation, and the onset of severe OFT defects. The requirement for Wnt/β-catenin signals is directly supported by the inventors discovery that a decrease in the proliferative capacity of isl1⁺ derivatives in the OFT of murine embryos that harbor a loss of β-catenin in isl1 lineage cells. Taken together, the inventors have demonstrated that defects in wnt/β-catenin pathways that control the renewal and differentiation of isl1⁺ cardiovascular progenitors in the AHF are related to the onset of severe OFT abnormalities, which constitute a major form of human congenital heart disease.

Wnt/β-Catenin Pathways and Cardiovascular Regenerative Medicine. One of the major limitations in cardiovascular regenerative medicine relates to the difficulty of expanding clonal cardiovascular progenitor populations, from either intact human tissue, or ES cell-based systems. In particular, the feasibility of utilizing human ES cells as a source for differentiated cardiac myocytes has been limited largely due to the inability to markedly enhance in vitro cardiogenesis, as less than 1% of the differentiated progeny enter cardiac lineages. The inventors have demonstrated that the manipulation of Wnt signals can be used for the isolation, cloning and expression of rare human Isl1+ cardiovascular progenitors from either ES or intact heart tissue. As the inventors have discovered that inhibition of GSK-3, by BIO treatment markedly increased the number of human Isl1+ progenitors, it demonstrates the wnt/β-catenin pathway has an evolutionary conserved role in the renewal and expansion of Isl1+ progenitors from a variety of mammalian origins, such as but not limited to human and rodent origins. Because the activation of wnt/β-catenin pathway been demonstrated to be effective in increasing the number of human Isl1+ progenitors, a similar renewal of Isl1+ progenitors is expected when the wnt/β-catenin signalling pathway is increased or activated in Isl1+ progenitors from other sources, such as other human progenitors or cells from other tissue, such as cardiac tissue.

### REFERENCES

The references cited herein and throughout the application are incorporated herein by reference.
Akashi, K., Kondo, M., Cheshier, S., Shizuru, J., Gandy, K., Domen, J., Mebius, R., Traver, D., and Weissman, I. L. (1999). Lymphoid development from stem cells and the common lymphocyte progenitors. Cold Spring Harb Symp Quant Biol 64, 1-12.
Akashi, K., Traver, D., Miyamoto, T., and Weissman, I. L. (2000). A clonogenic common myeloid progenitor that gives rise to all myeloid lineages. Nature 404, 193-197.
Banziger, C., Soldini, D., Schutt, C., Zipperlen, P., Hausmann, G., and Basler, K. (2006). Wntless, a conserved membrane protein dedicated to the secretion of Wnt proteins from signaling cells. Cell 125, 509-522.
Bartscherer, K., Pelte, N., Ingelfinger, D., and Boutros, M. (2006). Secretion of Wnt ligands requires Evi, a conserved transmembrane protein. Cell 125, 523-533.
Biben, C., Weber, R., Kesteven, S., Stanley, E., McDonald, L., Elliott, D. A., Barnett, L., Koentgen, F., Robb, L., Feneley, M., and Harvey, R. P. (2000). Cardiac septal and valvular dysmorphogenesis in mice heterozygous for mutations in the homeobox gene Nkx2-5. Circ Res 87, 888-895.
Brott, B. K., and Sokol, S. Y. (2005). A vertebrate homolog of the cell cycle regulator Dbf4 is an inhibitor of Wnt signaling required for heart development. Dev Cell 8, 703-715.
Cai, C. L., Liang, X., Shi, Y., Chu, P. H., Pfaff, S. L., Chen, J., and Evans, S. (2003). Isl1 identifies a cardiac progenitor population that proliferates prior to differentiation and contributes a majority of cells to the heart. Dev Cell 5, 877-889.
Chien, K. R., and Karsenty, G. (2005). Longevity and lineages: toward the integrative biology of degenerative diseases in heart, muscle, and bone. Cell 120, 533-544.
DasGupta, R., and Fuchs, E. (1999). Multiple roles for activated LEF/TCF transcription complexes during hair follicle development and differentiation. Development 126, 4557-4568.
Ding, S., Wu, T. Y., Brinker, A., Peters, E. C., Hur, W., Gray, N. S., and Schultz, P. G. (2003). Synthetic small molecules that control stem cell fate. Proc Natl Acad Sci USA 100, 7632-7637.
Ding, V. D., Qureshi, S. A., Szalkowski, D., Li, Z., Biazzo-Ashnault, D. E., Xie, D., Liu, K., Jones, A. B., Moller, D. E., and Zhang, B. B. (2002). Regulation of insulin signal transduction pathway by a small-molecule insulin receptor activator. Biochem J 367, 301-306.
Dodou, E., Verzi, M. P., Anderson, J. P., Xu, S. M., and Black, B. L. (2004). Mef2c is a direct transcriptional target of ISL1 and GATA factors in the anterior heart field during mouse embryonic development. Development 131, 3931-3942
Dodou, E., Xu, S. M., and Black, B. L. (2003). mef2c is activated directly by myogenic basic helix-loop-helix proteins during skeletal muscle development in vivo. Mech Dev 120, 1021-1032.
Dominguez, I., and Green, J. B. (2001). Missing links in GSK3 regulation. Dev Biol 235, 303-313.
Eisenberg, L. M., and Eisenberg, C. A. (2006). Wnt signal transduction and the formation of the myocardium. Dev Biol 293, 305-315.
Frame, S., and Cohen, P. (2001). GSK3 takes centre stage more than 20 years after its discovery. Biochem J 359, 1-16.
Harada, N., Tamai, Y., Ishikawa, T., Sauer, B., Takaku, K., Oshima, M., and Taketo, M. M. (1999). Intestinal polyposis in mice with a dominant stable mutation of the beta-catenin gene. EMBO J. 18, 5931-5942.
Glass, D. A., 2nd, Bialek, P., Ahn, J. D., Starbuck, M., Patel, M. S., Clevers, H., Taketo, M. M., Long, F., McMahon, A. P., Lang, R. A., and Karsenty, G. (2005). Canonical Wnt signaling in differentiated osteoblasts controls osteoclast differentiation. Dev Cell 8, 751-764.
Kattman, S. J., Huber, T. L., and Keller, G. M. (2006). Multipotent flk-1+ cardiovascular progenitor cells give rise to the cardiomyocyte, endothelial, and vascular smooth muscle lineages. Dev Cell 11, 723-732.
Knockaert, M., Blondel, M., Bach, S., Leost, M., Elbi, C., Hager, G. L., Nagy, S. R., Han, D., Denison, M., Ffrench, M., et al. (2004). Independent actions on cyclin-dependent kinases and aryl hydrocarbon receptor mediate the antiproliferative effects of indirubins. Oncogene 23, 4400-4412.
Kunick, C., Lauenroth, K., Leost, M., Meijer, L., and Lemcke, T. (2004). 1-Azakenpaullone is a selective inhibitor of glycogen synthase kinase-3 beta. Bioorg Med Chem Lett 14, 413-416.
Laugwitz, K. L., Moretti, A., Lam, J., Gruber, P., Chen, Y., Woodard, S., Lin, L. Z., Cai, C. L., Lu, M. M., Reth, M., et al. (2005). Postnatal isl1+ cardioblasts enter fully differentiated cardiomyocyte lineages. Nature 433, 647-653.
Liu, Y., Asakura, M., Inoue, H., Nakamura, T., Sano, M., Niu, Z., Chen, M., Schwartz, R. J., and Schneider, M.D. (2007). Sox17 is essential for the specification of cardiac mesoderm in embryonic stem cells. Proc. Natl. Acad. Sci. USA 104, 3859-3864.
Logan, C. Y., and Nusse, R. (2004). The Wnt signaling pathway in development and disease. Annu Rev Cell Dev Biol 20, 781-810.
Marvin, M. J., Di Rocco, G., Gardiner, A., Bush, S. M., and Lassar, A. B. (2001). Inhibition of Wnt activity induces heart formation from posterior mesoderm. Genes Dev 15, 316-327.
Meijer, L., Skaltsounis, A. L., Magiatis, P., Polychronopoulos, P., Knockaert, M., Leost, M., Ryan, X. P., Vonica, C. A., Brivanlou, A., Dajani, R., et al. (2003). GSK-3-selective inhibitors derived from Tyrian purple indirubins. Chem Biol 10, 1255-1266.
Metzger, J. M., Lin, W. I., Johnston, R. A., Westfall, M. V., and Samuelson, L. C. (1995). Myosin heavy chain expression in contracting myocytes isolated during embryonic stem cell cardiogenesis. Circ Res 76, 710-719.
Moretti, A., Caron, L., Nakano, A., Lam, J. T., Bernshausen, A., Chen, Y., Qyang, Y., Bu, L., Sasaki, M., Martin-Puig, S., et al. (2006). Multipotent embryonic isl1+ progenitor cells lead to cardiac, smooth muscle, and endothelial cell diversification. Cell 127, 1151-1165.

Nakamura, T., Sano, M., Zhou, S., and Schneider, M. D. (2003). A Wnt and β-catenin-dependent pathway for mammalian cardiac myogenesis. Proc. Natl. Acad. Sci. USA 100, 5834-5839.
Naito, A. T., Shiojima, I., Akazawa, H., Hidaka, K., Morisaki, T., Kikuchi, A., and Komuro, I. (2006). Developmental stage-specific biphasic roles of wnt/β-catenin signaling in cardiomyogenesis and hematopoiesis. Proc Natl Acad Sci USA 103, 19812-19817.
Plotkin, B., Kaidanovich, O., Talior, I., and Eldar-Finkelman, H. (2003). Insulin mimetic action of synthetic phosphorylated peptide inhibitors of glycogen synthase kinase-3. J Pharmacol Exp Ther 305, 974-980.
Polychronopoulos, P., Magiatis, P., Skaltsounis, A. L., Myrianthopoulos, V., Mikros, E., Tarricone, A., Musacchio, A., Roe, S. M., Pearl, L., Leost, M., et al. (2004). Structural basis for the synthesis of indirubins as potent and selective inhibitors of glycogen synthase kinase-3 and cyclin-dependent kinases. J Med Chem 47, 935-946.
Reya, T., Duncan, A. W., Ailles, L., Domen, J., Scherer, D. C., Willert, K., Hintz, L., Nusse, R., and Weissman, I. L. (2003). A role for Wnt signalling in self-renewal of haematopoietic stem cells. Nature 423, 409-414.
Sato, N., Meijer, L., Skaltsounis, L., Greengard, P., and Brivanlou, A. H. (2004). Maintenance of pluripotency in human and mouse embryonic stem cells through activation of Wnt signaling by a pharmacological GSK-3-specific inhibitor. Nat Med 10, 55-63.
Scadden, D. T. (2006). The stem-cell niche as an entity of action. Nature 441, 1075-1079.
Singh, A. M., Li, F. Q., Hamazaki, T., Kasahara, H., Takemaru, K., and Terada, N. (2007). Chibby, an antagonist of the Wnt/beta-catenin pathway, facilitates cardiomyocyte differentiation of murine embryonic stem cells. Circulation 115, 617-626.
Schneider, V. A., and Mercola, M. (2001). Wnt antagonism initiates cardiogenesis in Xenopus laevis. Genes Dev 15, 304-315.
Soriano, P. (1999). Generalized lacZ expression with the ROSA26 Cre reporter strain. Nat Genet. 21, 70-71.
Sun, Y., Liang, X., Najafi, N., Cass, M., Lin, L., Cai, C. L., Chen, J., and Evans, S. M. (2007). Islet 1 is expressed in distinct cardiovascular lineages, including pacemaker and coronary vascular cells. Dev. Biol. 304, 286-296
Tzahor, E., and Lassar, A. B. (2001). Wnt signals from the neural tube block ectopic cardiogenesis. Genes Dev 15, 255-260.
Vintersten, K., Monetti, C., Gertsenstein, M., Zhang, P., Laszlo, L., Biechele, S., and Nagy, A. (2004). Mouse in red: red fluorescent protein expression in mouse ES cells, embryos, and adult animals. Genesis 40, 241-246.
Verzi, M. P., McCulley, D. J., De Val, S., Dodou, E., and Black, B. L. (2005). The right ventricle, outflow tract, and ventricular septum comprise a restricted expression domain within the secondary/anterior heart field. Dev. Biol. 287, 134-145
Waldo, K. L., Kumiski, D. H., Wallis, K. T., Stadt, H. A., Hutson, M. R., Platt, D. H., and Kirby, M. L. (2001). Conotruncal myocardium arises from a secondary heart field. Development 128, 3179-3188.
Willert, K., Brown, J. D., Danenberg, E., Duncan, A. W., Weissman, I. L., Reya, T., Yates, J. R., 3rd, and Nusse, R. (2003). Wnt proteins are lipid-modified and can act as stem cell growth factors. Nature 423, 448-452.
Wu, S. M., Fujiwara, Y., Cibulsky, S. M., Clapham, D. E., Lien, C. L., Schultheis s, T. M., and Orkin, S. H. (2006). Developmental origin of a bipotential myocardial and smooth muscle cell precursor in the mammalian heart. Cell 127, 1137-1150.
Wu, X., Ding, S., Ding, Q., Gray, N. S., and Schultz, P. G. (2002). A small molecule with osteogenesis-inducing activity in multipotent mesenchymal progenitor cells. J Am Chem Soc 124, 14520-14521.
Wu, X., Walker, J., Zhang, J., Ding, S., and Schultz, P. G. (2004). Purmorphamine induces osteogenesis by activation of the hedgehog signaling pathway. Chem Biol 11, 1229-1238.
Xu, H., Morishima, M., Wylie, J. N., Schwartz, R. J., Bruneau, B. G., Lindsay, E. A., and Baldini, A. (2004). Tbx1 has a dual role in the morphogenesis of the cardiac outflow tract. Development 131, 3217-3227
Yin, T., and Li, L. (2006). The stem cell niches in bone. J Clin Invest 116, 1195-1201.
El-Badawi and Schenk, 1967
Mikawa, T. (1999). Cardiac lineages. In Heart Development (eds. Harvey R. and Rosenthal N.) Academic Press, 19-33

1. A method for inducing a cell to enter an islet 1+ lineage, the method comprising;
i. culturing a cell in the presence of a mesenchymal cell feeder layer;
ii. contacting the cell and/or the mesenchymal cell feeder layer with at least one wnt inhibitory agent, wherein the wnt inhibitory agent inhibits a Wnt/β-catenin signalling pathway in the cell; and
iii. culturing the cell for a sufficient period of time to promote the entry of the cell into the islet 1+ lineage;
wherein inhibition of a Wnt/β-catenin pathway in the cell induces it to enter the islet 1+ lineage.

2. The method of claim 1, wherein a cell that has entered the islet 1+ lineage is a multipotent islet 1+ progenitor.

3. The method of claim 1, wherein the multipotent islet 1+ progenitor is also positive for Nkx2.5, Isl1 and flk1.

4. The method of claim 2, wherein the multipotent islet 1+ progenitor is capable of multi-lineage differentiation, into a endothelial lineage, a cardiac lineage, a smooth muscle lineage, a myocyte lineage, a neural lineage, a autonomic lineage.

5.-6. (canceled)

7. The method of claim 1, wherein the cell is a stem cell, a progenitor cell or a mesoderm progenitor cell.

8. (canceled)

9. The method of claim 1, wherein the cell is a genetically modified cell.

10. The method of claim 9, wherein the genetically modified cell comprises a nucleic acid sequence encoding at least one wnt inhibitory agent, wherein the nucleic acid sequence encoding the wnt inhibitory agent is operatively linked to a regulatory sequence or promoter sequence.

11.-12. (canceled)

13. The method of claim 1, wherein the mesenchymal cell feeder layer is a cardiac mesenchymal cell (CMC) feeder layer.

14. The method of claim 1, wherein the mesenchymal cell feeder layer is a genetically modified mesenchymal cell feeder layer.

15. The method of claim 14, wherein the genetically modified mesenchymal cell feeder layer comprises a nucleic acid sequence encoding at least one wnt inhibitory agent, wherein the nucleic acid sequence encoding the wnt inhibitory agent is operatively linked to a regulatory sequence.

16. The method of claim 1, wherein the wnt inhibitory agent is selected from the group consisting of; a nucleic acid, protein, small molecule, antibody, an aptamer, a nucleic acid encoding a protein or fragment thereof, small inhibitory nucleic acid molecules, siRNA, shRNA, miRNA, antisense oligonucleic acids (ODNs), DNA or nucleic acid analogues, peptide-nucleic acid (PNA), pcPNA, locked nucleic acid (LNA) and analogues thereof.

17.-19. (canceled)

20. The method of claim 1, wherein the wnt inhibitory agent inhibits Wnt or Wnt3 or β-catenin.

21. The method of claim 1, wherein the wnt inhibitory agent inhibits Wls/Evi, Frizzled, Dsh (disheveled), LRP-5, LRP-6, Dally, Dally-like, PAR1, β-cateninin, TCF, lef-1 or Frodo.

22. (canceled)

23. The method of claim 1, wherein the wnt inhibitory agent is an RNAi agent which inhibits the RNA transcript of Wls/Evi or is a RNAi agent corresponds to SEQ ID NO:1 (siWLS-A) or SEQ ID NO:2 (siWLS-B).

24. (canceled)

25. The method of claim 1, wherein the wnt inhibitory agent is a nucleic acid encoding a protein or fragment thereof, or a protein of fragment thereof selected from the group of proteins consisting of: Dickkopf-1 (DKK1), WIF-1, cerberus, secreted frizzled-related proteins (sFRP), sFRP-1, sFRP-2, collagen 18 (collagen XVIII), endostatin, carboxypeptidase Z, receptor tyrosine kinase, corin, Dgl, Dapper, pertussis toxin, naked, Frz-related proteins or LRP lacking the intracellular domain.

26. (canceled)

27. The method of claim 20, wherein a wnt inhibitor agent which inhibits β-catenin is selected from the group consisting of; protein phosphatase 2A (PP2A), chibby, promtin 52, Nemo/LNK kinase, MHG homobox factors, XSox17, HBP1, APC, Axin, disabled-2 (dab-2) and gruncho (grg).

28. The method of claim 1, wherein the wnt inhibitory agent increases the activity and/or expression of GSK-3 and/or GSK3β or is a peptide of GSK3β.

29.-34. (canceled)

35. The method of claim 1, wherein the cell is derived or obtained from tissue.

36. The method of claim 1, wherein the tissue is human tissue.

37. (canceled)

38. The method of claim 35, wherein the tissue is cardiac tissue, fibroblasts, cardiac fibroblasts, circulating endothelial progenitors, pancreas, liver, adipose tissue, bone marrow, kidney, bladder, palate, umbilical cord, amniotic fluid, dermal tissue, skin, muscle, spleen, placenta, bone, neural tissue or epithelial tissue.

39. (canceled)

40. The method of claim 35, wherein the tissue is from a subject with an acquired or congenital cardiac heart defect, disease, disorder or dysfunction.

41.-89. (canceled)

90. A clonal cell line produced by the method set forth in any of the claims 1-40.

91. (canceled)

92. A method of enhancing cardiac function in a subject, the method comprising administering to the subject a composition comprising islet 1+ progenitors produced by the methods set forth in claim 1, wherein the composition comprising islet 1+ progenitors enhances cardiac function in the subject.

93. The method of claim 92, further defined as:
(i) obtaining a cell from the subject;
(ii) promoting the entry of the cell into the Islet 1+ lineage according to claim 1; and
(iii) transplanting the islet 1+ progenitors from step (ii) or their progeny into a subject, in amounts effective to treat a disorder characterized by insufficient cardiac function.

94. The method of claim 93, further comprising an additional step of differentiating the islet 1+ progenitors from step (ii) into desired cardiac lineages before transplanting the islet 1+ progenitors or their progeny into the subject.

95. The method of claim 92, wherein the subject suffers from a disorder characterized by insufficient cardiac function or suffers from a disorder selected from the group consisting of; congestive heart failure; myocardial infarction; tissue ischemia; cardiac ischemia; vascular diseases; acquired heart disease; congenital heart disease; arthlerscloerisis; cardiomyopathy; dysfunctional conduction systems; dysfunctional coronary arteries; pulmonary heart hypertension; hypertension.

96.-99. (canceled)

100. The method of claim 92, wherein the subject is a human.

101.-109. (canceled)

110. The method of claim 92, wherein the cells are harvested from the same subject to which the composition is administered.

111. The method of claim 92, wherein the cell is genetically modified such that the expression of at least one gene is altered in the cell before being transplanted in to the subject.

112.-130. (canceled)


SEQUENCE LISTING (8 sequences)

SEQ ID NO: 1 - DNA, 21 bp, Artificial Sequence
  Description of Artificial Sequence Synthetic oligonucleotide
cacaaatcctttctacagtat

SEQ ID NO: 2 - DNA, 19 bp, Artificial Sequence
  Description of Artificial Sequence Synthetic oligonucleotide
gggttaccgtgatgatatg

SEQ ID NO: 3 - protein, 12 aa, Artificial Sequence
  Description of Artificial Sequence Synthetic peptide
GKEAPPAPPQSP

SEQ ID NO: 4 - DNA, 2814 bp, Mus musculus
gaattcatgtcttacggtcaaggcagagggcccagcgccactgcagccgcgccacctcccagggccgggccagcccaggcgtccgcgctctcggggtggactccccccgctgcgcgctcaagccggcgatggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtaggagagctcctaacacgggagcagggttcattccgaggggcaaggttcctacctgggggcggggttcctacttggaggggtctcttacttggggactcggttcttacttgagggcggagatcctacctgtgagggtctcatacctaaggacccggtttctgccttcagcctgggctcctatttgggatctgggttcctttttaggggagaagctcctgtctgggatacgggtttctgcccgagggtggggctccacttggggatggaattccaatttgggccggaagtcctacctcaatggcttggactcctctcttgacccgacagggctcaaatggagacaggtaagctactccctcaactaggtggggttcgtgcggatgggtgggaggggagagattagggtccctcctcccagaggcactgctctatctagatacatgagagggtgcttcagggtgggccctatttgggcttgaggatcccgtgggggcggggcttcaccccgactgggtggaacttttggagacccccttccactggggcaaggcttcactgaagactcatgggatggagctccacggaaggaggagttcctgagcgagcctgggctctgagcaggccatccagctcccatctggcccctttccagtcctggtgtaaggttcaacctgcaagcctcatctgcgcagagcaggatctcctggcagaatgaggcatggagaagaactcaggggtgataccaagacctaacaaaccccgtgcctgggtacctcttttaaagctctgcaccccttcttcaagggctttcctagtctccttggcagagctttcctgaggaagatttgcagtcccccagagttcaagtgaacacccatagaacagaacagactctatcctgagtagagagggttctctaggaatctctatggggactgctaggaaggatcctgggcatgacagcctcgtatgatagcctgcatccgctctgacacttaatactcagatctcccgggaaacccagctcatccggtccgtgatgtccatgccccaaatgcctcagagatgttgcctcactttgagttgtatgaacttcggagacatggggacacagtcaagccgcagagccagggttgtttcaggacccatctgattccccagagcctgctgttgaggcaatggtcaccagatccgttggccaccaccctgtcccgagcttctctagtgtctgtctggcctggaagtgaggtgctacatacagcccatctgccacaagagcttcctgattggtaccactgtgaaccgtccctccccctccagacaggggaggggatgtggccatacaggagtgtgcccggagagcgcggaaagaggaagagaggctgcacacgcgtggtgactgactgtcttctgcctggaactttgcgttcgcgcttgtaactttattttcaatgctgctatatccacccaccactggatttagacaaaagtgattttctttttttttttttcttttctttctatgaaagaaattattttagtttatagtatgtttgtttcaaataatggggaaagtaaaaagagagaaaaaaaaaaaaaaaaaaaaaaaaaaaaaaaaaaaaaaaaaa

SEQ ID NO: 5 - DNA, 1506 bp, Homo sapiens
gcgcttctgacaagcccgaaagtcatttccaatctcaagtggactttgttccaactattgggggcgtcgctccccctcttcatggtcgcgggcaaacttcctcctcggcgcctcttctaatggagccccacctgctcgggctgctcctcggcctcctgctcggtggcaccagggtcctcgctggctacccaatttggtggtccctggccctgggccagcagtacacatctctgggctcacagcccctgctctgcggctccatcccaggcctggtccccaagcaactgcgcttctgccgcaattacatcgagatcatgcccagcgtggccgagggcgtgaagctgggcatccaggagtgccagcaccagttccggggccgccgctggaactgcaccaccatagatgacagcctggccatctttgggcccgtcctcgacaaagccacccgcgagtcggccttcgttcacgccatcgcctcggccggcgtggccttcgccgtcacccgctcctgcgccgagggcacctccaccatttgcggctgtgactcgcatcataaggggccgcctggcgaaggctggaagtggggcggctgcagcgaggacgctgacttcggcgtgttagtgtccagggagttcgcggatgcgcgcgagaacaggccggacgcgcgctcggccatgaacaagcacaacaacgaggcgggccgcacgactatcctggaccacatgcacctcaaatgcaagtgccacgggctgtcgggcagctgtgaggtgaagacctgctggtgggcgcagcctgacttccgtgccatcggtgacttcctcaaggacaagtatgacagcgcctcggagatggtagtagagaagcaccgtgagtcccgaggctgggtggagaccctccgggccaagtactcgctcttcaagccacccacggagagggacctggtctactacgagaactcccccaacttttgtgagcccaacccagagacgggttcctttggcacaagggaccggacttgcaatgtcacctcccacggcatcgatggctgcgatctgctctgctgtggccggggccacaacacgaggacggagaagcggaaggaaaaatgccactgcatcttccactggtgctgctacgtcagctgccaggagtgtattcgcatctacgacgtgcacacctgcaagtagggcaccagggcgctgggaaggggtgaagtgtgtggctgggcggattcagcgaagtctcatgggaagcaggacctagagccgggcacagccctcagcgtcagacagcaaggaactgtcaccagccgcacgcgtggtaaatgacccagacccaactcgcctgtggacggggaggctctccctctctctcatcttacatttctcaccctactctggatggtgtgtggtttttaaagaagggggctttctttttagttctctagggtctgataggaacagacctgaggcttatctttgcacatgttaaagaaaaaaaaaaaaa

SEQ ID NO: 6 - DNA, 2932 bp, Homo sapiens
agctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacgg

SEQ ID NO: 7 - DNA, 1639 bp, Homo sapiens
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 8 - DNA, 3362 bp, Homo sapiens
aagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttagcaaaccctagccttgcttgttaaaatttttttttttttttttttaagaatatctgtaatggtactgactttgcttgctttgaagtagctctttttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcatt